(19) United States
(12) Patent Application Publication
(10) Pub. No.: US 2022/0348565 A1
(43) Pub. Date: Nov. 3, 2022

(54) PHARMACEUTICAL COMPOUNDS

(71) Applicant: SENTINEL ONCOLOGY LIMITED, Cambridge (GB)

(72) Inventors: Robert George BOYLE, Cambridge (GB); Meriel Ruth MAJOR, Cambridge (GB); Stuart TRAVERS, Cambridge (GB); David Winter WALKER, Cambridge (GB); Michal CZYZEWSKI, Dunham (GB); Derek John LONDESBROUGH, Hartlepool (GB); Julian Scott NORTHEN, South Shields (GB); Stefania SANTONI, Newcastle uponTyne (GB)

(73) Assignee: SENTINEL ONCOLOGY LIMITED, Cambridge (GB)

(21) Appl. No.: 17/754,200

(22) PCT Filed: Sep. 25, 2020
(86) PCT No.: PCT/EP2020/076933

§ 371 Date: Mar. 25, 2022

(30) Foreign Application Priority Data
Sep. 26, 2019 (GB) 1913921.1

(51) Int. Cl. C07D 413/02; C07D 403/02; A61P 35/00

(52) U.S. Cl. CPC C07D 413/02; C07D 403/02; A61P 35/00

(57) ABSTRACT

The invention provides a composition of matter which:

(i) consists of at least 90% by weight of an atropisomer (2A) and 0-10% by weight of an atropisomer of formula (2B); or
(ii) consists of at least 90% by weight of an atropisomer (2B) and 0-10% by weight of an atropisomer of formula (2A);
wherein the atropisomer of formula (2A) and the atropisomer of formula (2B) are represented by:

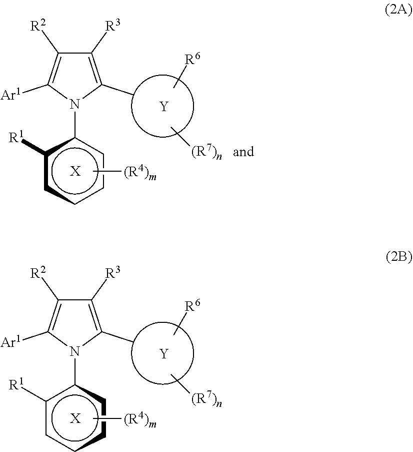

or are pharmaceutically acceptable salts or tautomers thereof, wherein ring X is a benzene or pyridine ring; ring Y is selected from a benzene ring, a pyridine ring and a thiophene ring; R¹ is trifluoromethyl; R² is hydrogen; R³ is hydrogen; m is 0 or 1; n is 0, 1 or 2; Ar¹ is a monocyclic aromatic ring selected from benzene and pyridine; each monocyclic aromatic ring being unsubstituted or substituted with 1 or 2 substituents R⁵ as defined herein; and R⁴; R⁵ when present, R⁶ and R⁷ independently selected from various substituents as defined herein.

Also provided are individual atropisomers, pharmaceutical compositions and the uses of the atropisomers and compositions are inhibitors of PLK1- and PLK4 kinases, for example in the treatment of cancers.

This invention relates to atropisomers of tri-aryl pyrrole derivatives and their analogues, methods for their preparation, pharmaceutical compositions containing them and their use in treating diseases such as cancer.

### BACKGROUND OF THE INVENTION

The protein expressed by the normal KRAS gene performs an essential function in normal tissue signalling. The mutation of a KRAS gene by a single amino acid substitution, and in particular a single nucleotide substitution, is responsible for an activating mutation which is an essential step in the development of many cancers. The mutated protein that results is implicated in various malignancies, including lung adenocarcinoma, mucinous adenoma, ductal carcinoma of the pancreas and colorectal carcinoma. Uke other members of the Ras family, the KRAS protein is a GTPase and is involved in many signal transduction pathways.

KRAS acts as a molecular on/off switch. Once it is turned on, it recruits and activates proteins necessary for the propagation of growth factor and other receptors' signal such as c-Raf and PI-3 Kinase. Normal KRAS binds to GTP in the active state and possesses an intrinsic enzymatic activity which cleaves the terminal phosphate of the nucleotide converting it to GDP. Upon conversion of GTP to GDP, KRAS is turned off. The rate of conversion is usually slow but can be sped up dramatically by an accessory protein of the GTPase-activating protein (GAP) class, for example RasGAP. In turn KRAS can bind to proteins of the Guanine Nucleotide Exchange Factor (GEF) class, for example SOS1, which forces the release of bound nucleotide. Subsequently, KRAS binds GTP present in the cytosol and the GEF is released from ras-GTP. In mutant KRAS, its GTPase activity is directly removed, rendering KRAS constitutively in the active state. Mutant KRAS is often characterised by mutations in codons 12, 13, 61 or mixtures thereof.

The viability of cancer cells carrying a mutant KRAS is known to be dependent on Polo-Uke Kinase 1 (PLK1) and it has been shown that silencing PLK1 leads to the death of cells containing mutant KRAS (see Luo et al., Cell. 2009 May 29; 137(5): 835-848). Compounds that inhibit PLK1 should therefore be useful in treating cancers that arise from KRAS mutations, but current kinase inhibitors designed to bind to the conserved ATP-binding domain of PLK1 may be too unselective versus other kinases to access this mode-of-action (see for example Elsayed et al., Future Med. Chem. (2019) 11(12), 1383-1386).

PLK1 is a serine/threonine kinase consisting of 603 amino acids and having a molecular weight of 66 kDa and is an important regulator of the cell cycle. In particular, PLK1 is important to mitosis and is involved in the formation of and the changes in the mitotic spindle and in the activation of CDK/cyclin complexes during the M-phase of the cell cycle.

All Polo-like kinases contain an N-terminal Serine/Threonine kinase catalytic domain and a C-terminal region that contains one or two Polo-boxes (Lowery et al., Oncogene, (2005), 24, 248-259). For Polo-like kinases 1, 2, and 3, the entire C-terminal region, including both Polo-boxes, functions as a single modular phosphoserine/threonine-binding domain known as the Polo-box domain (PBD). In the absence of a bound substrate, the PBD inhibits the basal activity of the kinase domain. Phosphorylation-dependent binding of the PBD to its ligands releases the kinase domain, while simultaneously localizing Polo-like kinases to specific subcellular structures.

It has been shown (Reindl et al., Chemistry & Biology, 15, 459-466, May 2008) that, because PLKL1 localizes to its intracellular anchoring sites via its polo-box domain, the action of PLK1 can be inhibited by small molecules which interfere with its intracellular localization by inhibiting the function of the PBD.

Tumour protein p53 functions as a tumour suppressor and plays a role in apoptosis, genomic stability and inhibition of angiogenesis. It is known that tumours with both p53-deficiency and high PLK1 expression may be particularly sensitive to PLK1 inhibitors (Yim et al., Mutat Res Rev Mutat Res, (2014). 761, 31-39).

The evidence in the literature thus suggests that small molecules that bind to and inhibit the function of the PBD should be effective inhibitors of PLK1 kinase and therefore should also be useful in the treatment of cancers arising from KRAS and/or p53 mutations. In particular, since PBD domains only reside in PLKs, the potential for inhibitors designed to this domain to have greater selectivity over previous ATP-competitive inhibitors, may enable a greater ability to target KRAS mutant and p53 deficient cancers.

The identification and development of drugs for treating primary brain cancers has proved to be particularly challenging. Targeted cancer therapies, and in particular therapies using protein kinase inhibitors, have been a major focus for pharmaceutical and biotechnology companies (Nature Reviews Clinical Oncology 2016, 13, 209-227). However, although over thirty kinase inhibitors have been approved for use in oncology, none of these have been for the treatment of primary brain cancer. A particular problem has been that most of the approved kinase inhibitor oncology drugs lack the necessary drug substance qualities to achieve the brain exposure needed if they are to be of use in the treatment of brain cancer [JMC 2016, 59(22), 10030-10066].

The alkylating agent temozolomide (Temodar®, Temodal®) is currently the first line treatment for the brain cancer glioblastoma multiforme and is frequently used in combination with radiation therapy. However, drug resistance is a major problem in the management of glioblastoma and therefore limits the usefulness of temozolomide. At the present time, therefore, malignant glioblastoma remains incurable.

Polo like kinase 1 (PLK1) is overexpressed in a range of tumour types including glioblastoma multiforme (Translational Oncology 2017, 10, 22-32). Furthermore, recent studies have shown that PLK1 drives checkpoint adaptation and resistance to temozolomide in glioblastoma multiforme [Oncotarget 2017, 8, 15827-15837].

Ependymomas are tumours of the brain and spinal cord with current standard of care limited to surgery and radiation. PLK1 has been implicated in Ependymomas and inhibitors of PLK1 are active against Ependymoma cell lines [Gilbertson et. al., Cancer Cell (2011) 20, 384-399].

PLK1 has also been investigated as a target for Diffuse Intrinsic Pontine Glioma (DIPG), a high grade, aggressive childhood brain tumour [Amani et al. BMC Cancer (2016) 16, 647 and Cancer Biology and Therapy (2018) 19, 12, 1078-1087]

More specifically, inhibition of PLK1 has been shown to enhance temozolomide efficacy in IDH1 mutant gliomas [Oncotarget, (2017) 8, 9, 15827-15837] and to inhibit tumour growth in an MMR-deficient, temozolomide-resistant glioblastoma xenograft model [Mol Cancer Ther; 17(12) December 2018]

In the cases above, current inhibitors lack sufficient brain exposure.

Compounds that inhibit PLKL1, but without inducing drug resistance, and which exhibit good brain exposure would be expected to be useful in the treatment of glioblastoma multiforme and other brain cancers.

PLK4 is a polo-like kinase family member of the serine/threonine kinases that plays a critical role in centrosome duplication, acting as a central regulator of centriole duplication (Bettencourt-Dias, Curr Biol. 2005 15(24); 2199-207). PLK4 dependent alterations in centrosomes can lead to asymmetric chromosome segregation at mitosis, which can trigger cell death after chromosome mis-segregation and mitotic defects.

PLK4 is aberrantly expressed in human cancers and is implicated in tumorigenesis and metastasis. As such PLK4 has been highlighted as a promising target for cancer therapy (Zhao, J Canc Res Clin Oncol., 2019).

PLK4 is overexpressed in many cancers including rhabdoid tumours, medulloblastoma and other embryonal tumours of the brain (Pediatr Blood Cancer. 2017), as well as breast, lung, melanoma, gastric, colorectal, pancreatic and ovarian cancer. Elevated or hyperactivated PLK4 is associated with poor survival rates in cancer patients, including ovarian, breast and lung cancers (Zhao, J Canc Res Clin Oncol., 2019).

PLK4 inhibition has been studied for the treatment of glioblastoma multiforme and it has been demonstrated that PLK4 plays a critical role in the regulation of temozolomide chemosensitivity. The combination of temozolomide with inhibition of PLK4 in glioblastoma PDX models has been shown to enhance the anti-tumor effects compared to temozolomide alone (Cancer Letters, Vol 443, 2019, 91-107).

PLK4 is reported to cooperate with p53 inactivation in cancer development, and it is predicted that cancers with PLK4 overexpression and p53 deficiency are prone to form tumours (Sercin, 2016; Nat Cell Biol 18:100-110). Therefore, compounds that inhibit PLK4 activity would be anticipated to be useful in the treatment of p53 mutant cancers.

Inhibition of PLK4 results in anti-tumour activity in lung cancer, with activity seen in cancers bearing wildtype and mutant KRAS (Kawakami, PNAS 2018, 115(8) 1913-18). Therefore, compounds that inhibit PLK4 activity would be anticipated to be useful in the treatment of KRAS mutant cancers.

Current PLK4 inhibitors act at the kinase active site and are not optimal for brain penetration (Int. J. Mol. Sci. 2019, 20, 2112). Therefore, compounds that inhibit PLK4 PBD but also exhibit good brain exposure would be anticipated to be useful in the treatment of glioblastoma multiforme and other brain cancers

Our earlier International patent application WO2018/197714 discloses compounds of the formula (0):

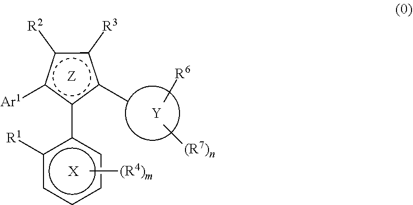

in which ring X is a benzene or pyridine ring, ring Y is a benzene, pyridine, thiophene or furan ring, Ar¹ is an optionally substituted benzene, pyridine, thiophene or furan ring, and R¹ to R⁴, R⁶, R⁷ are hydrogen or various substituents. The compounds are described as having anti-cancer activity and having good brain exposure after oral dosing, making them good candidates for the treatment of brain cancers. The compounds are active against glioblastoma cell lines and are believed to act as inhibitors of the Polo Box Domain of PLK1 kinase. It is also disclosed that the compounds are active against mutant-RAS cancer cell lines (such as HCT116) and should also be useful in the treatment of cancers arising from KRAS mutations.

### THE INVENTION

It has now been found that compounds of the type disclosed in our earlier application, wherein R¹ is a substituent of the size of a methyl group or larger, and in particular a trifluoromethyl group, form atropisomers. Atropisomers are stereoisomers resulting from hindered rotation about a single bond axis where the energy barrier to rotation barrier is sufficiently high to allow for the isolation of the individual rotational isomers; see LaPlante et al., J. Med. Chem., 54:7005-7022 (2011).

Atropisomers can be classified into three categories based on the amount of energy needed for the chiral axis to racemize via rotation and the length of time required for racemization to occur. Class 1 atropisomers possess barriers to rotation around the chiral axis of <84 kJ/mol (20 kcal/mol) and racemize over a time period measured in minutes or less at room temperature; Class 2 atropisomers possess a barrier to rotation between 84 and 117 kJ/mol (20-28 kcal/mol) and racemize over a time period measured in hours to months at room temperature; and Class 3 atropisomers possess a barrier to rotation >117 kJ/mol (28 kcal/mol) and racemize over a period of time measured in years at room temperature.

Atropisomers can be classified using the Cahn-Ingold-Prelog R and S system which is illustrated by (S)-6,6′-dinitrobiphenyl-2,2′-dicarboxylic acid shown in FIG. 1.

In this system, the nearest substituents either side of the aryl-aryl bond are assigned a priority in the order a-b-c-d. As the substituents a, b and c are in an anticlockwise arrangement, the atropisomer is the S isomer. In the corresponding R isomer, the substituents a, b and c are in a clockwise arrangement.

Atropisomer compounds of the invention are sufficiently stable to be isolated and characterised and have been found not to racemize to any significant extent even when heated to temperatures of up to 80° C. for a period of 10 days. The atropisomers of the invention can therefore be classified as Class 3 atropisomers. It believed that the atropisomerism arises because steric interactions between the substituent R¹ and the aromatic rings Ar¹ and Y prevent rotation about the bond between the rings Z and X.

Individual atropisomers of a given pair have been found to have significantly different biological properties. Thus, typically, one atropisomer of a pair is significantly more active against certain cancer targets than the other atropisomer of the pair.

According to a first Embodiment (Embodiment 1.1), the invention provides:

(i) a composition of matter consisting of at least 90% by weight of an atropisomer (1A) and 0-10% by weight of an atropisomer of formula (1B); or
(ii) a composition of matter consisting of at least 90% by weight of an atropisomer (1B) and 0-10% by weight of an atropisomer of formula (1A);
wherein the atropisomer of formula (1A) and the atropisomer of formula (1B) are represented by:

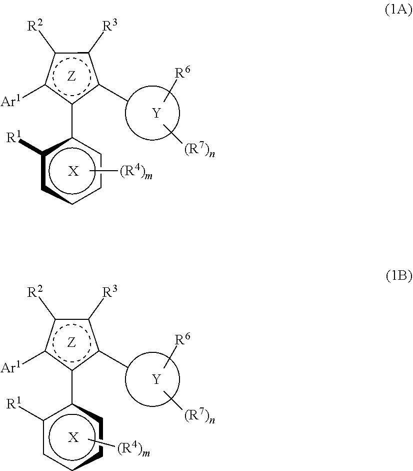

or are pharmaceutically acceptable salts or tautomers thereof, wherein
Z is a 5-membered heteroaryl ring containing one or two nitrogen ring members and optionally one further heteroatom ring member selected from N and O;
ring X is 6 membered carbocyclic or heterocyclic aromatic ring containing 0, 1 or 2 nitrogen heteroatom ring members;
ring Y is a 6 membered carbocyclic ring or a 5- or 6-membered heterocyclic aromatic ring containing 1 or 2 heteroatom ring members selected from N, O and S;
Ar¹ is a monocyclic 5- or 6-membered aromatic ring, optionally containing 0, 1 or 2 heteroatom ring members selected from N, O and S and being optionally substituted with one or more substituents R⁵;
m is 0, 1 or 2;
n is 0, 1 or 2;
R¹ is selected from:
chlorine;
bromine;
hydroxyl;
cyano;
carboxyl;
C(O)O(Hyd¹);
CONH₂;
amino;
—(Hyd²)NH;
(Hyd²)₂N; and
a C₁₋₅ hydrocarbon group where 0, 1 or 2 of the carbons in the hydrocarbon group are replaced with a heteroatom selected from N, O and S, the hydrocarbon group being optionally substituted with one or more fluorine atoms;
   Hyd¹, Hyd^{1a}, Hyd^{1b}, Hyd², Hyd^{2a}, Hyd^{2b} and Hyd^{2c} are the same or different and are C₁₋₄ hydrocarbon groups;
   R² is selected from hydrogen and a C₁₋₄ hydrocarbon group;
   R³ is selected from hydrogen and a C₁₋₄ hydrocarbon group;
   R⁴ is selected from:
fluorine;
chlorine;
bromine;
hydroxyl;
cyano;
carboxyl;
C(O)O(Hyd^{1a});
CONH₂;
amino;
—(Hyd^{2a})NH;
(Hyd^{2a})₂N; and
a C₁₋₅ hydrocarbon group where 0, 1 or 2 of the carbons in the hydrocarbon group are replaced with a heteroatom selected from N, O and S, the hydrocarbon group being optionally substituted with one or more fluorine atoms;
   R⁵ is selected from halogen; O—Ar²; cyano, hydroxy; amino; Hyd^{1b}-SO₂— and a non-aromatic C₁₋₈ hydrocarbon group where 0, 1 or 2 but not all of the carbons in the hydrocarbon group are optionally replaced with a heteroatom selected from N, O and S and where the hydrocarbon group is optionally substituted with one or more fluorine atoms;
   Ar² is a phenyl, pyridyl or pyridone group optionally substituted with 1 or 2 substituents selected from halogen; cyano and a C₁₋₄ hydrocarbon group optionally substituted with one or more fluorine atoms;
   R⁶ is selected from halogen, cyano, nitro and a group Q¹-R^{a}—R^{b};
   Q¹ is absent or is a C₁₋₆ saturated hydrocarbon linker;
   R^{a} is absent or is selected from O; C(O); C(O)O; CONR^{c}; N(R^{c})CO; N(R^{c})CONR^{c}, NRc; S; SO; SO₂; SO₂NR^{c}; and NR^{c}SO₂;
   R^{b} is selected from:
hydrogen;
a C₁₋₈ non-aromatic hydrocarbon group where 0, 1 or 2 of the carbon atoms in the hydrocarbon group are replaced with a heteroatom selected from N and O, the C₁₋₈ non-aromatic hydrocarbon group being optionally substituted with one or more substituents selected from fluorine and a group Cyc¹; and
a group Cyc¹;
   Cyc¹ is a non-aromatic 4-7 membered carbocyclic or heterocyclic ring group containing 0, 1 or 2 heteroatom ring members selected from N, O and S and being optionally substituted with one or more substituents selected from hydroxyl; amino; (Hyd^{2c})NH; (Hyd^{2c})₂N; and a C₁₋₅ hydrocarbon group where 0, 1 or 2 of the carbons in the hydrocarbon group are replaced with a heteroatom selected from N, O and S, the hydrocarbon group being optionally substituted with one or more fluorine atoms or by a 5- or 6-membered heteroaryl group containing 1 or 2 heteroatom ring members selected from N and O;
   R^{c} is selected from hydrogen and a C₁₋₄ non-aromatic hydrocarbon group; and
   R⁷ is independently selected from R⁴.

In formulae (1A) and (1B), Z is a 5-membered heteroaryl ring containing one or two nitrogen ring members and optionally one further heteroatom ring member selected from N and O.

It will be appreciated that when the 5-membered heteroaryl ring Z contains a second heteroatom ring member, for example when it is a pyrazole or isaxazole, one or both of R² and R³ will be absent. Accordingly, in each of the above and following aspects and embodiments where the 5-membered heteroaryl ring is other than a pyrrole, the definitions are to be taken as including compounds wherein one or both of R² and R³ are absent.

Particular and preferred aspects and embodiments of the invention are set out below in Embodiments 1.2 to 1.191.
1.2 A composition of matter according to Embodiment 1.1 provided that the composition of matter is other than one containing:
   (i) an atropisomer wherein R¹ is methyl, and R⁴ is a 4-cyano or 4-carbamoyl group;
   (ii) an atropisomer wherein R⁶ is hydroxy, methoxymethyl or unsubstituted or fluoro-substituted C₁₋₈ alkoxy (e.g. trifluoromethoxy);
   (iii) an atropisomer wherein the ring Z is an isoxazole ring and Ar¹ is an unsubstituted 4-pyridyl group attached to the isoxazole 3-position; and R² and R³ are both absent; or
   (iv) an atropisomer wherein Z is an isoxazole ring and R⁴ is an azetidin-4-yloxy group.
1.3 A composition of matter according to Embodiment 1.1 or Embodiment 1.2 which is other than a pyrrole substituted at each of the 1, 2 and 3 positions thereof with a substituted phenyl or pyridyl ring.
1.4 A composition of matter according to any one of Embodiments 1.1 to 1.3 wherein the ring Z is other than a 1,2,3-trisubstituted pyrrole ring.
1.5 A composition of matter according to any one of Embodiments 1.1 to 1.4 wherein the ring Z is other than an imidazole ring.
1.6 A composition of matter according to any one of Embodiments 1.1 to 1.5 wherein the ring Z is other than a 1,2,4 triazole ring.
1.7 A composition of matter according to any one of Embodiments 1.1 to Embodiment 1.6 wherein Z is a heteroaryl ring containing a nitrogen ring member and optionally one further heteroatom ring member selected from N and O; or Z is a triazole ring.
1.8 A composition of matter according to Embodiment 1.7 wherein Z is selected from pyrrole, isoxazole, imidazole, pyrazole and triazole rings.
1.9 A composition of matter according to Embodiment 1.8 wherein Z is selected from pyrrole, pyrazole and isoxazole rings.
1.10 A composition of matter according to Embodiment 1.9 wherein Z is a pyrrole ring.
1.11 A composition of matter according to Embodiment 1.10 wherein ring X is attached to the nitrogen atom of the pyrrole ring.
1.12 A composition of matter according to Embodiment 1.9 wherein Z is a pyrazole ring.
1.13 A composition of matter according Embodiment 1.12 wherein ring X is attached to a carbon atom of the pyrazole ring.
1.14 A composition of matter according to Embodiment 1.12 or Embodiment 1.13 wherein ring Y is attached to a carbon atom of the pyrazole ring.
1.15 A composition of matter according to Embodiment 1.12 or Embodiment 1.13 wherein ring Y is attached to a nitrogen atom of the pyrazole ring.
1.16 A composition of matter according to any one of Embodiments 1.12 to 1.15 wherein Ar¹ is attached to a carbon atom of the pyrazole ring.
1.17 A composition of matter according to Embodiment 1.9 wherein Z is an isoxazole ring.
1.18 A composition of matter according to Embodiment 1.17 wherein ring X is attached to the 4-position of the isoxazole ring.
1.19 A composition of matter according to Embodiment 1.17 or Embodiment 1.18 wherein ring Y is attached to the 5-position of the isoxazole ring.
1.20 A composition of matter according to any one of Embodiments 1.17 to 1.19 wherein Ar¹ is attached to the 3-position of the isoxazole ring.
1.21 A composition of matter according to any one of Embodiments 1.1 to 1.20 wherein the ring X is a benzene, pyridine or pyrimidine ring.
1.22 A composition of matter according to Embodiment 1.21 wherein the ring X is a benzene ring or pyridine ring.
1.23 A composition of matter according to Embodiment 1.22 wherein the ring X is a benzene ring.
1.24 A composition of matter according to Embodiment 1.22 wherein the ring X is a pyridine ring.
1.25 A composition of matter according to any one of Embodiments 1.21, 1.22 and 1.24 wherein the pyridine ring is a 2-pyridine ring.
1.26 A composition of matter according to any one of Embodiments 1.21, 1.22 and 1.24 wherein the pyridine ring is a 3-pyridine ring.
1.27 A composition of matter according to any one of Embodiments 1.21, 1.22 and 1.24 wherein the pyridine ring is a 4-pyridine ring.
1.28 A composition of matter according to any one of Embodiments 1.21, 1.22 and 1.24 wherein the pyridine ring is a 2-pyridine or 3-pyridine ring.
1.29 A composition of matter according to any one of Embodiments 1.1 to 1.28 wherein R¹ is selected from:
   chlorine;
   bromine;
   hydroxyl;
   cyano;
   carboxyl;
   amino;
   —(Hyd²)NH;
   (Hyd²)₂N;
   a C₁₋₅ hydrocarbon group where 0, 1 or 2 of the carbons in the hydrocarbon group are replaced with a heteroatom selected from N, O and S, the hydrocarbon group being optionally substituted with one or more fluorine atoms.
1.30 A composition of matter according to Embodiment 1.29 wherein R¹ is selected from:
   chlorine;
   bromine;
   hydroxyl;
   carboxyl;
   amino;
   methylamino;
   dimethylamino;
   a C₁₋₅ hydrocarbon group where 0, 1 or 2 of the carbons in the hydrocarbon group are replaced with a heteroatom selected from N, O and S, the hydrocarbon group being optionally substituted with one or more fluorine atoms.
1.31 A composition of matter according to Embodiment 1.29 wherein R¹ is selected from:
   chlorine;
   bromine;
   hydroxyl;
   amino;
   a C₁₋₅ hydrocarbon group where 0, 1 or 2 of the carbons in the hydrocarbon group are replaced with a heteroatom selected from N, O and S, the hydrocarbon group being optionally substituted with one or more fluorine atoms.
1.32 A composition of matter according to Embodiment 1.29 wherein R¹ is selected from:
   hydroxyl;
   amino; and
   a C₁₋₅ hydrocarbon group where 0, 1 or 2 of the carbons in the hydrocarbon group are replaced with a heteroatom selected from N, O and S, the hydrocarbon group being optionally substituted with one or more fluorine atoms.
1.33 A composition of matter according to Embodiment 1.29 wherein R¹ is selected from:
   hydroxyl;
   amino; and
   a C₁₋₄ hydrocarbon group where 0, 1 or 2 of the carbons in the hydrocarbon group are replaced with a heteroatom selected from N and O, the hydrocarbon group being optionally substituted with one or more fluorine atoms.
1.34 A composition of matter according to Embodiment 1.29 wherein R¹ is selected from a saturated C₁₋₄ hydrocarbon group where 0 or 1 or 2 of the carbons in the hydrocarbon group are replaced with a heteroatom selected from N and O, the hydrocarbon group being optionally substituted with one or more fluorine atoms.
1.35 A composition of matter according to Embodiment 1.29 wherein R¹ is selected from a saturated C₁₋₄ hydrocarbon group where 0 or 1 of the carbons in the hydrocarbon group are replaced with a heteroatom selected from N and O, the hydrocarbon group being optionally substituted with one or more fluorine atoms.
1.36 A composition of matter according to Embodiment 1.29 wherein R¹ is selected from a C₁₋₄ alkyl group where 0 or 1 of the carbons in the alkyl group are replaced with a heteroatom selected from N and O, the hydrocarbon group being optionally substituted with one or more fluorine atoms.
1.37 A composition of matter according to Embodiment 1.29 wherein R¹ is selected from hydroxyl; carboxyl; amino; a C₁₋₄ alkyl group which is optionally substituted with one or more fluorine atoms; a C₁₋₃ alkoxy group which is optionally substituted with one or more fluorine atoms; (dimethylamino)methyl and (methoxy)methyl.
1.38 A composition of matter according to Embodiment 1.29 wherein R¹ is selected from hydroxyl; carboxyl; amino; trifluoromethyl; (dimethylamino)methyl and (methoxy)methyl.
1.39 A composition of matter according Embodiment 1.29 wherein R¹ is a C₁₋₄ alkyl group optionally substituted with one or more fluorine atoms; or a C₁₋₃ alkoxy group optionally substituted with one or more fluorine atoms.
1.40 A composition of matter according Embodiment 1.29 wherein R¹ is a C₁₋₄ alkyl group substituted with one or more fluorine atoms.
1.41 A composition of matter according Embodiment 1.29 wherein R¹ is a C₁₋₂ alkyl group substituted with one or more fluorine atoms.
1.42 A composition of matter according Embodiment 1.41 wherein R¹ is a methyl group substituted with two or three fluorine atoms.
1.43 A composition of matter according to Embodiment 1.42 wherein R¹ is trifluoromethyl.
1.44 A composition of matter according to Embodiment 1.29 wherein R¹ is selected from hydrogen, trifluoromethyl, trifluoromethoxy, difluoromethyl or difluoromethoxy, hydroxyl, amino, carboxyl, (dimethylamino)methyl and (methoxy)methyl.
1.45 A composition of matter according to Embodiment 1.29 wherein R¹ is selected from trifluoromethyl; hydroxyl; amino; (dimethylamino)methyl and (methoxy)methyl.
1.46 A composition of matter according to any one of Embodiments 1.1 to 1.45 wherein m is 0 or 1.
1.47 A composition of matter according to any one of Embodiments 1.1 to 1.45 wherein m is 0.
1.48 A composition of matter according to any one of Embodiments 1.1 to 1.45 wherein m is 1.
1.49 A composition of matter according to any one of Embodiments 1.1 to 1.45 wherein m is 2.
1.50 A composition of matter according to any one of Embodiments 1.1 to 1.46, 1.48 and 1.49 wherein R⁴ is selected from:
   fluorine;
   chlorine;
   bromine;
   cyano; and
   a C₁₋₅ hydrocarbon group where 0, 1 or 2 of the carbons in the hydrocarbon group are replaced with a heteroatom selected from N, O and S, the hydrocarbon group being optionally substituted with one or more fluorine atoms.
1.51 A composition of matter according to Embodiment 1.50 wherein R⁴ is selected from:
   fluorine;
   chlorine;
   bromine;
   cyano; and
   a C₁₋₄ hydrocarbon group where 0 or 1 of the carbons in the hydrocarbon group are replaced with a heteroatom selected from N, O and S, the hydrocarbon group being optionally substituted with one or more fluorine atoms.
1.52 A composition of matter according to Embodiment 1.51 wherein R⁴ is selected from:
   fluorine;
   chlorine;
   bromine;
   cyano; and
   a C₁₋₄ alkyl group where 0 or 1 of the carbons in the alkyl group are replaced with a heteroatom selected from N and O, the alkyl group being optionally substituted with one or more fluorine atoms.
1.53 A composition of matter according to Embodiment 1.52 wherein R⁴ is selected from:
   fluorine;
   chlorine;
   bromine; and
   a C₁₋₄ alkyl group where 0 or 1 of the carbons in the alkyl group are replaced with a heteroatom O, the alkyl group being optionally substituted with one or more fluorine atoms.
1.54 A composition of matter according to Embodiment 1.53 wherein R⁴ is selected from fluorine; chlorine; bromine and C₁₋₄ alkyl.
1.55 A composition of matter according to Embodiment 1.54 wherein R⁴ is selected from fluorine; chlorine; and C₁₋₄ alkyl.
1.56 A composition of matter according to any one of Embodiments 1.1 to 1.55 wherein R² is selected from hydrogen and a saturated C₁₋₄ hydrocarbon group.
1.57 A composition of matter according to Embodiment 1.56 wherein R² is selected from hydrogen; C₁₋₄ alkyl; cyclopropyl and cyclopropylmethyl.
1.58 A composition of matter according to Embodiment 1.57 wherein R² is selected from hydrogen; C₁₋₃ alkyl and cyclopropyl.
1.59 A composition of matter according to Embodiment 1.58 wherein R² is selected from hydrogen; methyl and ethyl.
1.60 A composition of matter according to Embodiment 1.59 wherein R² is hydrogen or methyl.
1.61 A composition of matter according to Embodiment 1.60 wherein R² is hydrogen.
1.62 A composition of matter according to any one of Embodiments 1.1 to 1.61 wherein R³ is selected from hydrogen and a saturated C₁₋₄ hydrocarbon group.
1.63 A composition of matter according to Embodiment 1.62 wherein R³ is selected from hydrogen; C₁₋₄ alkyl; cyclopropyl and cyclopropylmethyl.
1.64 A composition of matter according to Embodiment 1.63 wherein R³ is selected from hydrogen; C₁₋₃ alkyl and cyclopropyl.
1.65 A composition of matter according to Embodiment 1.64 wherein R³ is selected from hydrogen; methyl and ethyl.
1.66 A composition of matter according to Embodiment 1.65 wherein R³ is hydrogen or methyl.
1.67 A composition of matter according to Embodiment 1.66 wherein R³ is hydrogen.
1.68 A composition of matter according to any one of Embodiments 1.1 to 1.67 wherein Ar¹ is a monocyclic aromatic ring selected from benzene; pyridine; pyrimidine; thiophene; and furan; each of the monocyclic aromatic rings being optionally substituted with one or more substituent R⁵.
1.69 A composition of matter according to Embodiment 1.68 wherein Ar¹ is a monocyclic aromatic ring selected from benzene; pyridine and pyrimidine; each of the monocyclic aromatic rings being optionally substituted with one or more substituent R⁵.
1.70 A composition of matter according to Embodiment 1.69 wherein Ar¹ is a monocyclic aromatic ring selected from benzene and pyridine; each of the monocyclic aromatic rings being optionally substituted with one or more substituent R⁵.
1.71 A composition of matter according to Embodiment 1.70 wherein Ar¹ is a benzene ring optionally substituted with one or more substituent R⁵.
1.72 A composition of matter according to Embodiment 1.70 wherein Ar¹ is a pyridine ring optionally substituted with one or more substituent R⁵.
1.73 A composition of matter according to any one of Embodiments 1.1 to 1.72 wherein the monocyclic aromatic ring Ar¹ is unsubstituted or is substituted with 1, 2, or 3 substituents R⁵.
1.74 A composition of matter according to Embodiment 1.73 wherein the monocyclic aromatic ring Ar¹ is unsubstituted or is substituted with 1 or 2 substituents R⁵.
1.75 A composition of matter according to Embodiment 1.74 wherein the monocyclic aromatic ring Ar¹ is unsubstituted or is substituted with 1 substituent R⁵.
1.76 A composition of matter according to Embodiment 1.75 wherein the monocyclic aromatic ring Ar¹ is substituted with 1 substituent R⁵.
1.77 A composition of matter according to Embodiment 1.75 wherein the monocyclic aromatic ring Ar¹ is unsubstituted.
1.78 A composition of matter according to Embodiment 1.74 wherein the monocyclic aromatic ring Ar¹ is substituted with 2 substituents R⁵.
1.79 A composition of matter according to any one of Embodiments 1.1 to 1.76 and 1.78 wherein R⁵ is selected from halogen; O—Ar²; cyano, Hyd^{1b}-SO₂— and a C₁₋₈ hydrocarbon group where 0, 1 or 2 but not all of the carbons in the hydrocarbon group are optionally replaced with a heteroatom selected from N, O and S and where the hydrocarbon group is optionally substituted with one or more fluorine atoms.
1.80 A composition of matter according to any one of Embodiments 1.1 to 1.76, 1.78 and 1.79 wherein Ar² is a phenyl or pyridyl group optionally substituted with 1 or 2 substituents selected from fluorine, chlorine, cyano and trifluoromethyl.
1.81 A composition of matter according to Embodiment 1.80 wherein Ar² is a phenyl group optionally substituted with 1 or 2 substituents selected from fluorine, chlorine, cyano and trifluoromethyl.
1.82 A composition of matter according to any one of Embodiments 1.1 to 1.76 and 1.78 to 1.80 wherein Hyd^{1b} is a saturated C₁₋₄ hydrocarbon group.
1.83 A composition of matter according to Embodiment 1.82 wherein Hyd^{1b} is selected from C₁₋₄ alkyl; cyclopropyl and cyclopropylmethyl.
1.84 A composition of matter according to Embodiment 1.78 wherein Hyd^{1b} is selected from methyl; ethyl; propyl; cyclopropyl and cyclopropylmethyl.
1.85 A composition of matter according to Embodiment 1.79 wherein Hyd^{1b} is selected from methyl; ethyl; propyl and cyclopropyl.
1.86 A composition of matter according to Embodiment 1.80 wherein Hyd^{1b} is selected from methyl and ethyl.
1.87 A composition of matter according to Embodiment 1.81 wherein Hyd^{1b} is methyl.
1.88 A composition of matter according to any one of Embodiments 1.1 to 1.76 and 1.78 wherein R⁵ is selected from bromine; fluorine; chlorine; cyano; phenoxy; C₁₋₄ alkylsulphonyl; C₁₋₄ alkoxy and C₁₋₄ alkyl wherein the C₁₋₄ alkoxy and C₁₋₄ alkyl are each optionally substituted with one or more fluorine atoms.
1.89 A composition of matter according to Embodiment 1.88 wherein R⁵ is selected from bromine; fluorine; chlorine; cyano; phenoxy; methylsulphonyl; methyl; ethyl; isopropyl; difluoromethyl; trifluoromethyl; methoxy; difluoromethoxy; and trifluoromethoxy.
1.90 A composition of matter according to Embodiment 1.89 wherein R⁵ is selected from bromine; fluorine; chlorine; cyano; phenoxy; methylsulphonyl; and isopropyl.
1.90A A composition of matter according to any one of Embodiments 1.1 to 1.90 wherein R⁵ is located at a para position on the monocyclic aromatic ring Ar¹.
1.91 A composition of matter according to Embodiment 1.90 or Embodiment 1.91 wherein R⁵ is selected from bromine; fluorine; chlorine; and cyano.
1.92 A composition of matter according to Embodiment 1.91 wherein R⁵ is selected from fluorine, chlorine and cyano.
1.93 A composition of matter according to Embodiment 1.92 wherein R⁵ is cyano.
1.94 A composition of matter according to Embodiment 1.93 wherein Ar¹ is 4-cyanophenyl.
1.95 A composition of matter according to Embodiment 1.92 wherein R⁵ is chlorine.
1.96 A composition of matter according to Embodiment 1.95 wherein Ar¹ is 4-chlorophenyl.
1.97 A composition of matter according to Embodiment 1.92 wherein R⁵ is fluorine.
1.98 A composition of matter according to Embodiment 1.97 wherein Ar¹ is 4-fluorophenyl.
1.99 A composition of matter according to any one of Embodiments 1.1 to 1.98 wherein the ring Y is a benzene, pyridine, pyrimidine, furan, thiophene or pyrrole ring.
1.100 A composition of matter according to Embodiment 1.99 wherein the ring Y is either a) a benzene ring, b) a pyridine ring or c) a thiophene ring.
1.101 A composition of matter according to Embodiment 1.100 wherein the ring Y is a benzene ring.
1.102 A composition of matter according to Embodiment 1.100 wherein the ring Y is a pyridine ring.
1.103 A composition of matter according to any one of Embodiments 1.1 to 1.102 wherein R⁶ is a group Q¹-R^{a}—R^{b}.
1.104 A composition of matter according to any one of Embodiments 1.1 to 1.103 wherein Q¹ has the formula (CR^{p}R^{q})ᵣ wherein r is 0, 1, 2, 3 or 4 and R^{p} and R^{q} are independently selected from hydrogen and methyl or R^{p} and R^{q} together with the carbon atom to which they are attached form a 3- or 4-membered saturated cyclic hydrocarbon ring, provided that the total number of carbons in Q¹ does not exceed 6.
1.105 A composition of matter according any one of Embodiments 1.1 to 1.104 wherein Q¹ is absent or is selected from CH₂, CH(CH₃), C(CH₃)₂, cyclopropane-1,1-diyl and cyclobutane-1,1-diyl.
1.106 A composition of matter according any Embodiment 1.105 wherein Q¹ is absent.
1.107 A composition of matter according to Embodiment 1.105 wherein Q¹ is a —CH₂— group.
1.108 A composition of matter according to any one of Embodiments 1.1 to 1.107 wherein R^{a} is absent or is selected from O; C(O); C(O)O; CONR^{c}; N(R^{c})CO; N(R^{c})CONR^{c}; NR^{c}; and SO₂.
1.109 A composition of matter according to Embodiment 1.108 wherein R^{a} is absent or is selected from O; CONR^{c}; N(R^{c})CO; N(R^{c})CONR^{c}, NR^{c} and SO₂.
1.110 A composition of matter according to Embodiment 1.108 wherein R^{a} is CONR^{c}.
1.111 A composition of matter according to Embodiment 1.108 wherein R^{a} is N(R^{c})CO.
1.112 A composition of matter according to Embodiment 1.108 wherein R^{a} is NR^{c}.
1.113 A composition of matter according to Embodiment 1.108 wherein R^{a} is absent.
1.114 A composition of matter according to Embodiment 1.108 wherein R^{a} is O.
1.115 A composition of matter according to Embodiment 1.108 wherein R^{a} is C(O).
1.116 A composition of matter according to Embodiment 1.108 wherein R^{a} is C(O)O.
1.117 A composition of matter according to Embodiment 1.108 wherein R^{a} is SO₂.
1.118 A composition of matter according to any one of Embodiments 1.1 to 1.117 wherein R^{b} is selected from:
   a C₁₋₈ non-aromatic hydrocarbon group where 0, 1 or 2 but not all of the carbon atoms in the hydrocarbon group are replaced with a heteroatom selected from N and O, the C₁₋₈ non-aromatic hydrocarbon group being optionally substituted with one or more substituents selected from fluorine and a group Cyc¹; and
   a group Cyc¹;
   provided that when R^{a} is C(O)O or CONR^{c}; then R^{b} is additionally selected from hydrogen.
1.119 A composition of matter according to Embodiment 1.118 wherein R^{b} is selected from:
   a C₁₋₈ non-aromatic hydrocarbon group where 0, 1 or 2 of the carbon atoms in the hydrocarbon group are replaced with a heteroatom selected from N and O, the C₁₋₈ non-aromatic hydrocarbon group being optionally substituted with one or more substituents selected from fluorine and a group Cyc¹; and
   a group Cyc¹.
1.120 A composition of matter according to Embodiment 1.119 wherein R^{b} is selected from:
   a C₁₋₈ non-aromatic hydrocarbon group where 0 or 1 but not all of the carbon atoms in the hydrocarbon group are replaced with a heteroatom selected from N and O, the C₁₋₈ non-aromatic hydrocarbon group being optionally substituted with one or more substituents selected from fluorine and a group Cyc¹; and
   a group Cyc¹.
1.121 A composition of matter according to Embodiment 1.120 wherein R^{b} is selected from:
   a C₁₋₈ non-aromatic hydrocarbon group where 0 or 1 but not all of the carbon atoms in the hydrocarbon group are replaced with a heteroatom selected from N and O, the C₁₋₈ non-aromatic hydrocarbon group being optionally substituted with a group Cyc¹; and
   a group Cyc¹.
1.122 A composition of matter according to Embodiment 1.121 wherein R^{b} is selected from:
   a C₁₋₈ non-aromatic hydrocarbon group wherein 1 of the carbon atoms in the hydrocarbon group is replaced with a heteroatom selected from N and O; and
   a group Cyc¹.
1.123 A composition of matter according to Embodiment 1.122 wherein R^{b} is selected from:
   a C₁₋₈ non-aromatic hydrocarbon group wherein 1 of the carbon atoms in the hydrocarbon group is replaced with a heteroatom N; and
   a group Cyc¹.
1.124 A composition of matter according to any one of Embodiments 1.1 to 1.117 wherein R^{b} is a C₁₋₈ non-aromatic hydrocarbon group where 0, 1 or 2 but not all of the carbon atoms in the hydrocarbon group are replaced with a heteroatom selected from N and O, the C₁₋₈ non-aromatic hydrocarbon group being optionally substituted with one or more substituents selected from fluorine and a group Cyc¹.
1.125 A composition of matter according to Embodiment 1.124 wherein R^{b} is selected from:
   a C₁₋₈ non-aromatic hydrocarbon group where 0 or 1 but not all of the carbon atoms in the hydrocarbon group are replaced with a heteroatom selected from N and O, the C₁₋₈ non-aromatic hydrocarbon group being optionally substituted with one or more substituents selected from fluorine and a group Cyc¹.
1.126 A composition of matter according to Embodiment 1.125 wherein R^{b} is selected from:
   a C₁₋₈ non-aromatic hydrocarbon group where 0 or 1 but not all of the carbon atoms in the hydrocarbon group are replaced with a heteroatom selected from N and O, the C₁₋₈ non-aromatic hydrocarbon group being optionally substituted with a group Cyc¹.
1.127 A composition of matter according to Embodiment 1.126 wherein R^{b} is selected from:
   a C₁₋₈ non-aromatic hydrocarbon group wherein 1 of the carbon atoms in the hydrocarbon group is replaced with a heteroatom selected from N and O.
1.128 A composition of matter according to any one of Embodiments 1.1 to 1.127 wherein R^{b} is selected from:
   a C₁₋₈ non-aromatic hydrocarbon group wherein 1 of the carbon atoms in the hydrocarbon group is replaced with a nitrogen heteroatom.
1.129 A composition of matter according to any one of Embodiments 1.118 to 1.128 wherein R^{b} is selected from:
   a C₁₋₈ non-aromatic hydrocarbon group wherein a carbon atom in the hydrocarbon group is replaced with a nitrogen heteroatom so as to form a terminal dimethyamino group.
1.130 A composition of matter according to any one of Embodiments 1.118 to 1.129 wherein the non-aromatic hydrocarbon group is acyclic.
1.131 A composition of matter according to any one of Embodiments 1.118 to 1.130 wherein the non-aromatic hydrocarbon group is saturated.
1.132 A composition of matter according to any one of Embodiments 1.118 to 1.131 wherein the non-aromatic hydrocarbon group contains 1 to 6 carbon atoms.
1.133 A composition of matter according to any one of Embodiments 1.118 to 1.132 wherein the non-aromatic hydrocarbon group contains 1 to 5 carbon atoms.
1.134 A composition of matter according to any one of Embodiments 1.118 to 1.133 wherein the non-aromatic hydrocarbon group contains 3 to 5 carbon atoms.
1.135 A composition of matter according to any one of Embodiments 1.1 to 1.126 wherein R^{b} is or contains a group Cyc¹.
1.136 A composition of matter according to Embodiment 1.128 wherein R^{b} is a group Cyc¹.
1.137 A composition of matter according to Embodiment 1.136 wherein Cyc¹ is a non-aromatic 4-7 membered carbocyclic or heterocyclic ring group containing 0, 1 or 2 heteroatom ring members selected from N and O and being optionally substituted with one or more substituents selected from hydroxyl; amino; mono-C₁₋₄ alkylamino; di-C₁₋₄ alkylamino; and a C₁₋₅ saturated hydrocarbon group where 0 or 1 but not all of the carbons in the hydrocarbon group are replaced with a heteroatom selected from N and O.
1.138 A composition of matter according to Embodiment 1.137 wherein Cyc¹ is a non-aromatic 4-7 membered heterocyclic ring group containing a nitrogen ring member and optionally second heteroatom ring member selected from N and O; the non-aromatic 4-7 membered heterocyclic ring group being optionally substituted with one or more substituents selected from hydroxyl; amino; mono-C₁₋₄ alkylamino; di-C₁₋₄ alkylamino; and a C₁₋₄ saturated hydrocarbon group where 0 or 1 but not all of the carbons in the hydrocarbon group are replaced with a heteroatom selected from N and O.
1.139 A composition of matter according to Embodiment 1.138 wherein Cyc¹ is a non-aromatic 5-6 membered heterocyclic ring group containing a nitrogen ring member and optionally second heteroatom ring member selected from N and O; the non-aromatic 5-6 membered heterocyclic ring group being optionally substituted with one or more substituents selected from hydroxyl; amino; mono-C₁₋₄ alkylamino; di-C₁₋₄ alkylamino; and a C₁₋₄ saturated hydrocarbon group where 0 or 1 but not all of the carbons in the hydrocarbon group are replaced with a heteroatom selected from N and O.
1.140 A composition of matter according to Embodiment 1.139 wherein Cyc¹ is a non-aromatic 5-6 membered heterocyclic ring group containing a nitrogen ring member and optionally second heteroatom ring member selected from N and O; the non-aromatic 5-6 membered heterocyclic ring group being optionally substituted with one or more substituents selected from hydroxyl; amino; mono-C₁₋₂ alkylamino; di-C₁₋₂ alkylamino; and a C₁₋₄ alkyl group where 0 or 1 but not all of the carbons in the alkyl group are replaced with a heteroatom selected from N and O.
1.141 A composition of matter according to any one of Embodiments 1.1 to 1.126, and 1.135 to 1.140 wherein Cyc¹ is a saturated ring.
1.142 A composition of matter according to Embodiment 1.141 wherein Cyc¹ is selected from pyrrolidine; piperidine; and piperazine; each of which is optionally substituted with one or more substituents selected from hydroxyl; amino; mono-C₁₋₂ alkylamino; di-C₁₋₂ alkylamino; and a C₁₋₄ alkyl group where 0 or 1 but not all of the carbons in the alkyl group are replaced with a heteroatom selected from N and O.
1.143 A composition of matter according to any one of Embodiments 1.1 to 1.112 and 1.118 to 1.142 wherein R^{c} is selected from hydrogen; methyl; ethyl; propyl; iso-propyl; cyclopropyl; cyclopropylmethyl; butyl; iso-butyl and cyclobutyl.
1.144 A composition of matter according to Embodiment 1.143 wherein R^{c} is selected from hydrogen and methyl.
1.145 A composition of matter according to Embodiment 1.144 wherein R^{c} is hydrogen.
1.146 A composition of matter according to Embodiment 1.144 wherein R^{c} is methyl.
1.147 A composition of matter according to any one of Embodiments 1.1 to 1.103 wherein R⁶ is selected from groups A to AL in the table below.

| | |
|---|---|
| 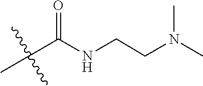 [SMILES: *C(=O)NCCN(C)C] | A |
| 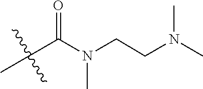 [SMILES: *C(=O)N(C)CCN(C)C] | B |
| 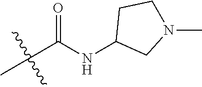 [SMILES: *C(=O)NC1CCN(C)C1] | C |
| 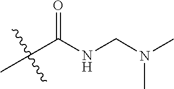 [SMILES: *C(=O)NCN(C)C] | D |
| 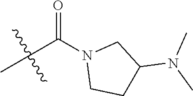 [SMILES: *C(=O)N1CCC(N(C)C)C1] | E |
| 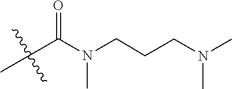 [SMILES: *C(=O)N(C)CCCN(C)C] | F |
| 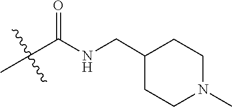 [SMILES: *C(=O)NCC1CCN(C)CC1] | G |
| 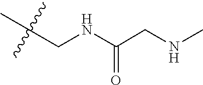 [SMILES: *CNC(=O)CNC] | H |
| 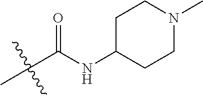 [SMILES: *C(=O)NC1CCN(C)CC1] | I |
| 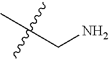 [SMILES: *CN] | J |
| 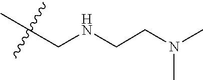 [SMILES: *CNCCN(C)C] | K |
| 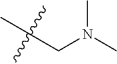 [SMILES: *CN(C)C] | L |
| 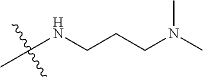 [SMILES: *NCCCN(C)C] | M |
| 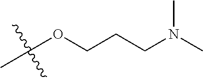 [SMILES: *OCCCN(C)C] | N |
| 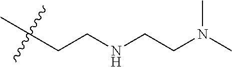 [SMILES: *CCNCCN(C)C] | O |
| 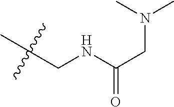 [SMILES: *CNC(=O)CN(C)C] | P |
| 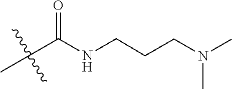 [SMILES: *C(=O)NCCCN(C)C] | Q |
| 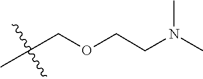 [SMILES: *COCCN(C)C] | R |
| 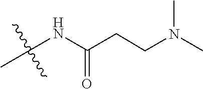 [SMILES: *NC(=O)CCN(C)C] | S |
| 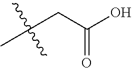 [SMILES: *CC(=O)O] | T |
| 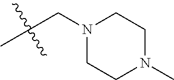 [SMILES: *CN1CCN(C)CC1] | U |
| —CO₂H | V |
| —SO₂NH₂ | W |
| 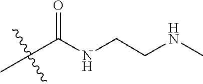 [SMILES: *C(=O)NCCNC] | X |
| 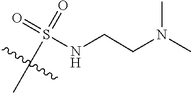 [SMILES: *S(=O)(=O)NCCN(C)C] | Y |
| 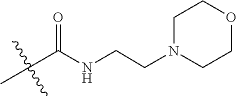 [SMILES: *C(=O)NCCN1CCOCC1] | Z |
| 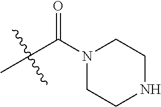 [SMILES: *C(=O)N1CCNCC1] | AA |
| 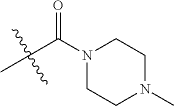 [SMILES: *C(=O)N1CCN(C)CC1] | AB |
| 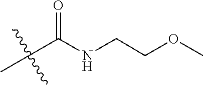 [SMILES: *C(=O)NCCOC] | AC |
| 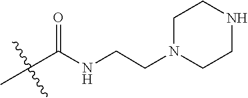 [SMILES: *C(=O)NCCN1CCNCC1] | AD |
| 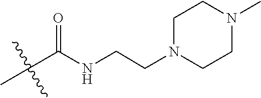 [SMILES: *C(=O)NCCN1CCN(C)CC1] | AE |
| 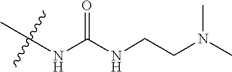 [SMILES: *NC(=O)NCCN(C)C] | AF |
| 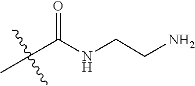 [SMILES: *C(=O)NCCN] | AG |
| 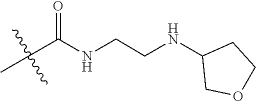 [SMILES: *C(=O)NCCNC1CCOC1] | AH |
| 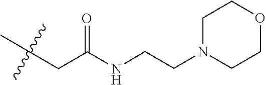 [SMILES: *CC(=O)NCCN1CCOCC1] | AI |
| 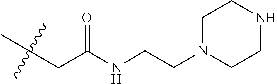 [SMILES: *CC(=O)NCCN1CCNCC1] | AJ |
| 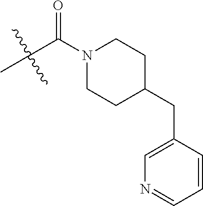 [SMILES: *C(=O)N1CCC(Cc2cccnc2)CC1] | AK |
| 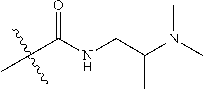 [SMILES: *C(=O)NCC(C)N(C)C] | AL |

1.148 A composition of matter according to Embodiment 1.147 wherein R⁶ is selected from groups A and Q.
1.149 A composition of matter according to Embodiment 1.148 wherein R⁶ is a group A.
1.149A A composition of matter according to any one of Embodiments 1.1 to 1.149 wherein n is 0 or 1.
1.149B A composition of matter according to Embodiments 1.149A wherein n is 0.
1.149C A composition of matter according to Embodiments 1.149A wherein n is 1.
1.149D A composition of matter according to any one of Embodiments 1.1 to 1.149A and 1.149C wherein R⁷ is selected from fluorine, chlorine and methoxy.
1.149E A composition of matter according to Embodiment 1.149D wherein R⁷ is selected from chlorine and methoxy.
1.149F A composition of matter according to Embodiment 1.149D wherein n is 1 and R⁷ is chlorine.
1.149G A composition of matter according to Embodiment 1.149D wherein n is 1 and R⁷ is methoxy.
1.150 A composition of matter according to any one of Embodiments 1.1 to 1.149 wherein: (i) when Y is a six membered ring, R⁶ is attached at the meta or para position thereof; or (ii) when Y is a five membered ring, R⁶ is attached to ring Y at a position which is not adjacent a ring member of Y to which ring Z is attached.
1.151 A composition of matter according to Embodiment 1.150 wherein Y is a six membered ring and R⁶ is attached at the meta or para position thereof.
1.152 A composition of matter according to Embodiment 1.151 wherein Y is a six membered ring and R⁶ is attached at the meta position thereof.
1.153 A composition of matter according to Embodiment 1.151 wherein Y is a six membered ring and R⁶ is attached at the para position thereof.
1.154 A composition of matter consisting of a least 90% by weight of an atropisomer (1A) and 0-10% by weight of an atropisomer of formula (1B); wherein the atropisomer of formula (1A) and the atropisomer of formula (1B) are represented by:

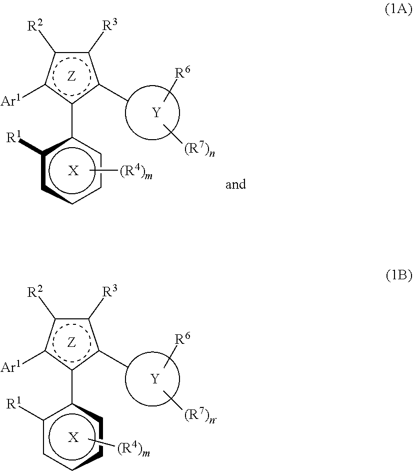

or are pharmaceutically acceptable salts or tautomers thereof, wherein R¹ to R⁷, Ar¹, m, n, X, Y and Z are as defined in any one of Embodiments 1.1 to 1.153.
1.155 A composition of matter according to Embodiment 1.154 consisting of at least 95% by weight of an atropisomer (1A), or a salt or tautomer thereof, and 0-5% by weight of an atropisomer of formula (1B), or a salt or tautomer thereof.
1.156 A composition of matter according to Embodiment 1.154 consisting of at least 96% by weight of an atropisomer (1A), or a salt or tautomer thereof, and 0-4% by weight of an atropisomer of formula (1B), or a salt or tautomer thereof.
1.157 A composition of matter according to Embodiment 1.154 consisting of at least 97% by weight of an atropisomer (1A), or a salt or tautomer thereof, and 0-3% by weight of an atropisomer of formula (1B), or a salt or tautomer thereof.
1.158 A composition of matter according to Embodiment 1.154 consisting of at least 98% by weight of an atropisomer (1A), or a salt or tautomer thereof, and 0-2% by weight of an atropisomer of formula (1B), or a salt or tautomer thereof.
1.159 A composition of matter according to Embodiment 1.154 consisting of at least 99% by weight of an atropisomer (1A), or a salt or tautomer thereof, and 0-1% by weight of an atropisomer of formula (1B), or a salt or tautomer thereof.
1.160 A composition of matter according to Embodiment 1.154 consisting of at least 99.5% by weight of an atropisomer (1A), or a salt or tautomer thereof, and 0-0.5% by weight of an atropisomer of formula (1B), or a salt or tautomer thereof.
1.161 A composition of matter consisting of a least 90% by weight of an atropisomer (1B) and 0-10% by weight of an atropisomer of formula (1A); wherein the atropisomer of formula (1A) and the atropisomer of formula (1B) are represented by:

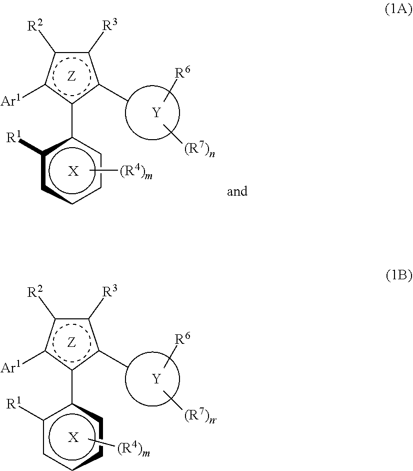

or are pharmaceutically acceptable salts or tautomers thereof, wherein R¹ to R⁷, Ar¹, m, n, X, Y and Z are as defined in any one of Embodiments 1.1 to 1.153.
1.162 A composition of matter according to Embodiment 1.161 consisting of at least 95% by weight of an atropisomer (1B), or a salt or tautomer thereof, and 0-5% by weight of an atropisomer of formula (1A), or a salt or tautomer thereof.
1.163 A composition of matter according to Embodiment 1.161 consisting of at least 96% by weight of an atropisomer (1B), or a salt or tautomer thereof, and 0-4% by weight of an atropisomer of formula (1A), or a salt or tautomer thereof.
1.164 A composition of matter according to Embodiment 1.161 consisting of at least 97% by weight of an atropisomer (1B), or a salt or tautomer thereof, and 0-3% by weight of an atropisomer of formula (1A), or a salt or tautomer thereof.
1.165 A composition of matter according to Embodiment 1.161 consisting of at least 98% by weight of an atropisomer (1B), or a salt or tautomer thereof, and 0-2% by weight of an atropisomer of formula (1A), or a salt or tautomer thereof.
1.166 A composition of matter according to Embodiment 1.161 consisting of at least 99% by weight of an atropisomer (1B), or a salt or tautomer thereof, and 0-1% by weight of an atropisomer of formula (1A), or a salt or tautomer thereof.
1.167 A composition of matter according to Embodiment 1.161 consisting of at least 99.5% by weight of an atropisomer (1B), or a salt or tautomer thereof, and 0-0.5% by weight of an atropisomer of formula (1A), or a salt or tautomer thereof.
1.168 A composition of matter:
   (i) consisting of at least 90% by weight of an atropisomer (2A) and 0-10% by weight of an atropisomer of formula (2B); or
   (ii) consisting of at least 90% by weight of an atropisomer (2B) and 0-10% by weight of an atropisomer of formula (2A);
   wherein the atropisomer of formula (2A) and the atropisomer of formula (2B) are represented by:

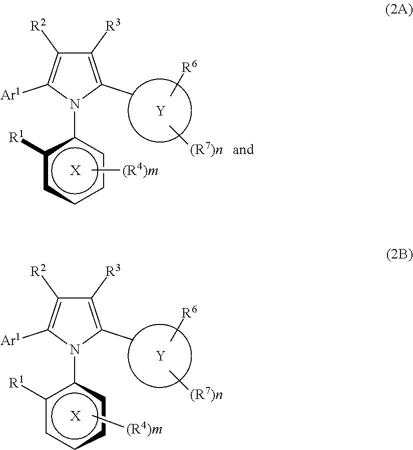

or are pharmaceutically acceptable salts or tautomers thereof, wherein R¹, R², R³, R⁴, R⁶, R⁷, Ar¹, X and Y are as defined in any one of Embodiments 1.1, 1.2, 1.8, 1.10, 1.11 and 1.21 to 1.153.
1.169 A composition of matter according to Embodiment 1.168 consisting of at least 95% by weight of an atropisomer (2A), or a salt or tautomer thereof, and 0-5% by weight of an atropisomer of formula (2B), or a salt or tautomer thereof.
1.170 A composition of matter according to Embodiment 1.168 consisting of at least 96% by weight of an atropisomer (2A), or a salt or tautomer thereof, and 0-4% by weight of an atropisomer of formula (2B), or a salt or tautomer thereof.
1.171 A composition of matter according to Embodiment 1.168 consisting of at least 97% by weight of an atropisomer (2A), or a salt or tautomer thereof, and 0-3% by weight of an atropisomer of formula (2B), or a salt or tautomer thereof.
1.172 A composition of matter according to Embodiment 1.168 consisting of at least 98% by weight of an atropisomer (2A), or a salt or tautomer thereof, and 0-2% by weight of an atropisomer of formula (2B), or a salt or tautomer thereof.
1.173 A composition of matter according to Embodiment 1.168 consisting of at least 99% by weight of an atropisomer (2A), or a salt or tautomer thereof, and 0-1% by weight of an atropisomer of formula (2B), or a salt or tautomer thereof.
1.174 A composition of matter according to Embodiment 1.168 consisting of at least 99.5% by weight of an atropisomer (2A), or a salt or tautomer thereof, and 0-0.5% by weight of an atropisomer of formula (2B), or a salt or tautomer thereof.
1.175 A composition of matter according to Embodiment 1.168 consisting of at least 95% by weight of an atropisomer (2B), or a salt or tautomer thereof, and 0-5% by weight of an atropisomer of formula (2A), or a salt or tautomer thereof.
1.176 A composition of matter according to Embodiment 1.168 consisting of at least 96% by weight of an atropisomer (2B), or a salt or tautomer thereof, and 0-4% by weight of an atropisomer of formula (2A), or a salt or tautomer thereof.
1.177 A composition of matter according to Embodiment 1.168 consisting of at least 97% by weight of an atropisomer (2B), or a salt or tautomer thereof, and 0-3% by weight of an atropisomer of formula (2A), or a salt or tautomer thereof.
1.178 A composition of matter according to Embodiment 1.168 consisting of at least 98% by weight of an atropisomer (2B), or a salt or tautomer thereof, and 0-2% by weight of an atropisomer of formula (2A), or a salt or tautomer thereof.
1.179 A composition of matter according to Embodiment 1.168 consisting of at least 99% by weight of an atropisomer (2B), or a salt or tautomer thereof, and 0-1% by weight of an atropisomer of formula (2A), or a salt or tautomer thereof.
1.180 A composition of matter according to any one of Embodiments 1.168 to 1.179 wherein:
   R¹ is selected from trifluoromethyl, hydroxyl, amino, (dimethylamino)methyl and (methoxy)methyl;
   R² is hydrogen;
   R³ is hydrogen;
   R⁴ is absent or is selected from chlorine, fluorine and C₁₋₄ alkyl;
   Ar¹ is phenyl or pyridyl optionally substituted with one or two substituents R⁵ selected from bromine, fluorine, chlorine, phenoxy, C₁₋₄ alkyl (e.g. isopropyl), C₁₋₄ alkylsulphonyl (e.g. methylsulphonyl) and cyano;
   X is selected from phenyl and pyridyl;
   m is 0 or 1;
   Y is selected from phenyl, pyridyl and thienyl;
   n is 0 or 1;
   R⁶ is selected from groups A to AM in Table 1 above; and
   R⁷ is selected from chlorine, fluorine and C₁₋₄ alkoxy (e.g. methoxy).
1.181 A composition of matter according to Embodiment 1.180 wherein:
   R¹ is trifluoromethyl;
   R² is hydrogen;
   R³ is hydrogen;
   Ar¹ is phenyl substituted with a substituent R⁵ selected from fluorine, chlorine and cyano;
   X is phenyl;
   m is 0;
   Y is phenyl or pyridyl;
   n is 0; and
   R⁶ is a group (A):

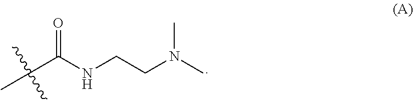 [SMILES: *C(=O)NCCN(C)C]

1.182 A composition of matter according to any one of Embodiments 1.1 and 1.154 to 1.179 wherein the atropisomer is an atropisomer of a compound of any one of Examples A-1 to A-8 and B-2 to B-107.
1.183 A composition of matter according to any one of Embodiments 1.1 and 1.154 to 1.179 wherein the atropisomer is an atropisomer of a compound of any one of Examples A-1 to A-8.
1.184 A composition of matter consisting of 99.5-100% by weight of a single atropisomer as defined in any one of Embodiments 1.1 to 1.183.
1.185 A composition of matter consisting of 99.9-100% by weight of a single atropisomer as defined in any one of Embodiments 1.1 to 1.183.
1.186 A single atropisomer having a chemical structure as defined in any one of Embodiments 1.1 to 1.183, said single atropisomer being unaccompanied by any other atropisomer, or being accompanied by no more than 0.5% by weight relative to the single atropisomer of any other atropisomer.
1.187 A single atropisomer having a chemical structure as defined in any one of Embodiments 1.1 to 1.183, said single atropisomer being unaccompanied by any other atropisomer, or being accompanied by no more than 0.25% by weight relative to the single atropisomer of any other atropisomer.
1.188 A single atropisomer having a chemical structure as defined in any one of Embodiments 1.1 to 1.183, said single atropisomer being unaccompanied by any other atropisomer, or being accompanied by no more than 0.1% by weight relative to the single atropisomer of any other atropisomer.
1.188A A single atropisomer according to Embodiment 1.188, which has the R configuration represented by formula (1), or is a salt thereof:

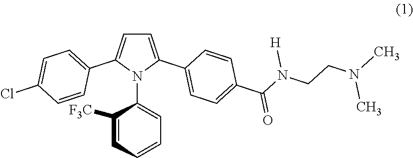

1.189 A composition of matter as defined in any one of Embodiments 1.1 to 1.185 or a single atropisomer as defined in any one of Embodiments 1.186 to 1.188A wherein each atropisomer is in the form of a salt.
1.190 A composition of matter as defined in any one of Embodiments 1.1 to 1.187 or a single atropisomer as defined in Embodiment 1.188 or 1.188A wherein each atropisomer is in the form of an acid addition salt.
1.191 A composition of matter as defined in any one of Embodiments 1.1 to 1.187 or a single atropisomer as defined in Embodiment 1.188 or 1.188A wherein each atropisomer is in a non-salt form.
1.192 A composition of matter as defined in any one of Embodiments 1.1 to 1.187 or a single atropisomer as defined in Embodiment 1.188 or 1.188A wherein each atropisomer is in the form of an acid addition salt (preferably having an approximately 1:1 salt ratio) formed with an acid selected from hydrochloric, methanesulfonic, maleic, malic, tartaric, p-toluenesulfonic, phosphoric and sulfuric acids.

A preferred acid addition salt of the invention is a 1:1 salt formed between the single atropisomer Compound (1) of Embodiment 1.88A and (+)-L-tartaric acid.

The (+)-L-tartaric acid is particularly advantageous in that it is a highly crystalline and stable solid taking up only surface moisture (<1% at 90% RH) with improved water solubility over the free base. These properties render it particularly suitable for pharmaceutical development.

Accordingly, in further embodiments (Embodiments 1.193 to 1.211), the invention provides:
1.193 (R)-2,4-[5-(4-chlorophenyl)-1-[2-(trifluoromethyl)-phenyl]pyrrol-2-yl]-N-[2-(dimethylamino)-ethyl]benzamide (+)-L-tartaric acid salt having an approximately 1:1 molar ratio between acid and base.
1.194 A (+)-L-tartaric acid salt of 2,4-[5-(4-chlorophenyl)-1-[2-(trifluoromethyl)-phenyl]pyrrol-2-yl]-N-[2 (dimethylamino)ethyl]benzamide having the formula (2):

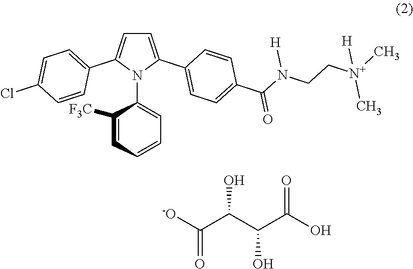

1.195 A (+)-L-tartaric acid salt of 2,4-[5-(4-chlorophenyl)-1-[2-(trifluoromethyl)-phenyl]pyrrol-2-yl]-N-[2 (dimethylamino)ethyl]benzamide, in which there is an approximately 1:1 molar ratio between acid and base and wherein the 2,4-[5-(4-chlorophenyl)-1-[2-(trifluoromethyl)-phenyl]pyrrol-2-yl]-N-[2 (dimethylamino)ethyl]-benzamide is in the form of a single atropisomer.
1.196 A (+)-L-tartaric acid salt according to Embodiment 1.195 wherein the single atropisomer is an atropisomer of formula (1) as defined in Embodiment 1.188A.
1.197 A (+)-L-tartaric acid salt according to Embodiment 1.195 wherein the single atropisomer is the R atropisomer of 2,4-[5-(4-chlorophenyl)-1-[2-(trifluoromethyl)-phenyl]pyrrol-2-yl]-N-[2 (dimethylamino)ethyl]benzamide.
1.198 A (+)-L-tartaric acid salt according to Embodiment 1.95 wherein the single atropisomer is characterised by any one or more of the following parameters:
   (i) X-ray crystallographic data substantially as described in Example 3 herein;
   (ii) a retention time of approximately 20 minutes (e.g. approximately 20.5 minutes) when determined by Chiral HPLC method 1 herein; and
   (iii) a specific optical rotation, when measured using the method described in Example 2 herein, of approximately −11.76°.
1.199 A (+)-L-tartaric acid salt according to Embodiment 1.195 wherein the single atropisomer is atropisomer A-2 as described in the Examples herein.
1.200 A (+)-L-tartaric acid salt according to Embodiment 1.195 wherein the (+)-L-tartaric acid salt is as described in the Examples herein.
1.201 A (+)-L-tartaric acid salt according to any one of Embodiments 1.193 to 1.200 which is in a crystalline form.
1.202 A (+)-L-tartaric acid salt according to Embodiment 1.201 which is an anhydrate.
1.203 A (+)-L-tartaric acid salt according to Embodiment 1.202 which is the anhydrate identified herein as Pattern B.
1.204 A (+)-L-tartaric acid salt according to Embodiment 1.201 which is a solvate.
1.205 A (+)-L-tartaric acid salt according to Embodiment 1.204 which is the solvate identified herein as Pattern A.
1.206 A composition of matter comprising the (+)-L-tartaric acid salt of any one of Embodiments 1.193 to 1.205 wherein either (a) the single atropisomer is the only atropisomer of 2,4-[5-(4-chlorophenyl)-1-[2-(trifluoromethyl)-phenyl]pyrrol-2-yl]-N-[2 (dimethylamino)ethyl]benzamide present in the composition or (b) there is less than 10% by molar amount, relative to the said single atropisomer, of any other atropisomer of 2,4-[5-(4-chlorophenyl)-1-[2-(trifluoromethyl)-phenyl]pyrrol-2-yl]-N-[2 (dimethylamino)ethyl]benzamide.
1.207 A composition of matter according to Embodiment 1.206 wherein either (a) the single atropisomer is the only atropisomer of 2,4-[5-(4-chlorophenyl)-1-[2-(trifluoromethyl)-phenyl]pyrrol-2-yl]-N-[2 (dimethylamino)ethyl]benzamide present in the composition or (b) there is less than 5% by molar amount, relative to the said single atropisomer, of any other atropisomer of 2,4-[5-(4-chlorophenyl)-1-[2-(trifluoromethyl)-phenyl]pyrrol-2-yl]-N-[2 (dimethylamino)ethyl]benzamide.
1.208 A composition of matter according to Embodiment 1.206 wherein either (a) the single atropisomer is the only atropisomer of 2,4-[5-(4-chlorophenyl)-1-[2-(trifluoromethyl)-phenyl]pyrrol-2-yl]-N-[2 (dimethylamino)ethyl]benzamide present in the composition or (b) there is less than 2% by molar amount, relative to the said single atropisomer, of any other atropisomer of 2,4-[5-(4-chlorophenyl)-1-[2-(trifluoromethyl)-phenyl]pyrrol-2-yl]-N-[2 (dimethylamino)ethyl]benzamide.
1.209 A composition of matter according to Embodiment 1.206 wherein either (a) the single atropisomer is the only atropisomer of 2,4-[5-(4-chlorophenyl)-1-[2-(trifluoromethyl)-phenyl]pyrrol-2-yl]-N-[2 (dimethylamino)ethyl]benzamide present in the composition or (b) there is less than 1.5% by molar amount, relative to the said single atropisomer, of any other atropisomer of 2,4-[5-(4-chlorophenyl)-1-[2-(trifluoromethyl)-phenyl]pyrrol-2-yl]-N-[2 (dimethylamino)ethyl]benzamide.
1.210 A composition of matter according to Embodiment 1.206 wherein either (a) the single atropisomer is the only atropisomer of 2,4-[5-(4-chlorophenyl)-1-[2-(trifluoromethyl)-phenyl]pyrrol-2-yl]-N-[2 (dimethylamino)ethyl]benzamide present in the composition or (b) there is less than 1% by molar amount, relative to the said single atropisomer, of any other atropisomer of 2,4-[5-(4-chlorophenyl)-1-[2-(trifluoromethyl)-phenyl]pyrrol-2-yl]-N-[2 (dimethylamino)ethyl]benzamide.
1.211 A composition of matter according to Embodiment 1.206 wherein either (a) the single atropisomer is the only atropisomer of 2,4-[5-(4-chlorophenyl)-1-[2-(trifluoromethyl)-phenyl]pyrrol-2-yl]-N-[2 (dimethylamino)ethyl]benzamide present in the composition or (b) there is less than 0.1% by molar amount, relative to the said single atropisomer, of any other atropisomer of 2,4-[5-(4-chlorophenyl)-1-[2-(trifluoromethyl)-phenyl]pyrrol-2-yl]-N-[2 (dimethylamino)ethyl]benzamide.

### Definitions

The terms “atropisomer compound(s)”, “atropisomer compound(s) of the invention”, “compound(s) of the formula (1)”, “compound(s)” and “compound(s) of the invention” and like terms may be used herein to refer to the compositions of matter and the atropisomers defined in any of Embodiments 1.1 to 1.211. Unless the context indicates otherwise, such terms may be taken as referring to any of the atropisomers of the formulae (1A), (1B), (2A) and (2B) and all sub-groups, preferences, embodiments and examples as defined herein. The term “compound of the formula (1)” may be used herein as a generic term covering the atropisomers of the formulae (1A), (1B), (2A) and (2B) and all sub-groups, preferences, embodiments and examples thereof, as well as mixtures of the atropisomers. It will be apparent from the context in which a reference to a compound of the formula (1) is made whether it refers to an individual atropisomers, composition of matter, or mixture of atropisomers.

The term ‘medicament’ as used herein refers to a pharmaceutical formulation that is of use in treating, curing or improving a disease or in treating, ameliorating or alleviating the symptoms of a disease. A pharmaceutical formulation comprises a pharmacologically active ingredient in a form not harmful to the subject it is being administered to and additional constituents designed to stabilise the active ingredient and affect its absorption into the circulation or target tissue.

Where the atropisomers defined in any one of Embodiments 1.1 to 1.188A contain ionisable groups, they may be presented in the form of salts, as defined in any one of Embodiments 1.189, 1.190 and 1.192 to 1.211.

For example, where the atropisomers contain a basic (e.g. nitrogen basic) group or atom, the atropisomers can be presented in the form of acid addition salts.

The salts can be synthesized from the parent compound by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free base form of the compound with the acid in water or in an organic solvent, or in a mixture of the two; generally, nonaqueous media such as ether, ethyl acetate, ethanol, isopropanol, or acetonitrile are used.

Acid addition salts (as defined in Embodiment 1.190) may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, p-toluenesulphonic, undecylenic and valeric acids, as well as acylated amino acids and cation exchange resins.

The salt forms of the compositions of matter or atropisomers of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the composition of matter or atropisomers of the invention, also form part of the invention.

Geometric Isomers and Tautomers

In addition to existing as atropisomers, the compositions of matter or atropisomers of the invention may contain other structural features that give rise to geometric isomerism, and tautomerism and references to the composition of matter or atropisomers as defined in Embodiments 1.1 to 1.211 include all geometric isomer and tautomeric forms. For the avoidance of doubt, where an atropisomer can exist in one of several geometric isomeric or tautomeric forms and only one is specifically described or shown, all others are nevertheless embraced by formulae (1A) (1B) or subgroups, subsets, preferences and examples thereof.

Optical Isomers

Where compounds of the invention contain one or more chiral centres in addition to the structural features giving rise to atropisomerism, references to the composition of matter or atropisomers include all optical isomeric forms thereof (e.g. enantiomers, epimers and diastereoisomers), either as individual optical isomers, or mixtures thereof (other than mixtures of atropisomers), unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the invention exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing an atropisomer having one or more chiral centres, wherein at least 55% (e.g. at least 60%, 65%, 70%, 75%, 80%, 85%, 90% or 95%) of the composition of matter or atropisomer of the formula (1) is present as a single optical isomer (e.g. enantiomer or diastereoisomer). In one general embodiment, 99% or more (e.g. substantially all) of the total amount of the composition of matter or atropisomer of the formula (1) may be present as a single optical isomer (e.g. enantiomer or diastereoisomer).

Isotopes

The composition of matter or atropisomers of the invention as defined in any one of Embodiments 1.1 to 1.211 may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ³C and ¹⁴C and ¹⁶O and ¹⁸O.

The isotopes may be radioactive or non-radioactive. In one embodiment of the invention, the composition of matter or atropisomers contain no radioactive isotopes. Such compounds are preferred for therapeutic use. In another embodiment, however, the composition of matter or atropisomer may contain one or more radioisotopes. Compounds containing such radioisotopes may be useful in a diagnostic context.

Solvates

The compositions of matter or atropisomers as defined in any one of Embodiments 1.1 to 1.211 may form solvates and anhydrates.

Particular solvates are solvates formed by the incorporation into the solid state structure (e.g. crystal structure) of the compositions of matter or atropisomers of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the composition of matter or atropisomers of the invention with a solvent or mixture of solvents containing the solvating solvent. Whether or not a solvate has been formed in any given instance can be determined by subjecting crystals of the composition of matter or atropisomer to analysis using well known and standard techniques such as thermogravimetric analysis (TGE), differential scanning calorimetry (DSC) and X-ray powder diffraction (XRPD).

The solvates can be stoichiometric or non-stoichiometric solvates.

Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

In addition to forming solvates, the compositions of matter, compounds or salts as defined in any one of Embodiments 1.1 to 1.211 may be provided in the form of an anhydrate. The term “anhydrate” as used herein refers to a solid particulate form which does not contain water (and preferably does not contain any other solvents) within its three-dimensional structure (e.g. crystalline form), although particles of the salt or compound may have water molecules attached to an outer surface thereof.

Prodrugs

The compounds, salts, compositions of matter or atropisomers as defined in any one of Embodiments 1.1 to 1.211 may be presented in the form of a pro-drug.

By “prodrugs” is meant for example any compound that is converted in vivo into a biologically active composition of matter or atropisomer, as defined in any one of Embodiments 1.1 to 1.211.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any hydroxyl groups present in the parent compound with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Methods for the Preparation of Compounds of the Invention

The compositions of matter and atropisomers of the invention can be prepared by separation of mixtures of atropisomers using chiral chromatography and in particular chiral HPLC.

Mixtures of atropisomers of the formula (1A), (1B), (2A), (2B) and the various sub-groups thereof can be prepared in accordance with synthetic methods well known to the skilled person. Unless stated otherwise, R¹-R⁷, Ar¹, X, Y and Z are as hereinbefore defined. In the following paragraphs relating to the preparation of mixtures of atropisomers, the mixtures are referred to generically as compounds of the formula (1).

Compounds of the formula (1) wherein Z is a pyrrole ring can be prepared by reacting a 1,4-dicarbonyl compound of formula (10) with an aminoaryl compound of formula (11) as shown in Scheme 1.

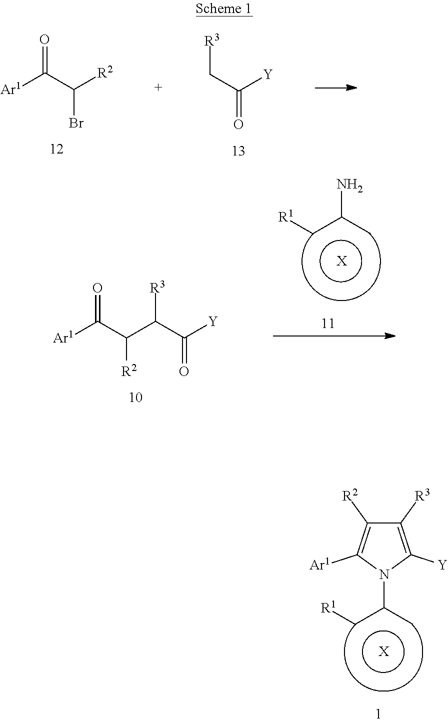

The starting material for the synthetic route shown in Scheme 1 is the 1-aryl-3-bromopropanone (12) with arylpropanone (13), which can both be obtained commercially.

The 1-aryl-2-bromoethanone (12), is reacted with arylpropanone (13) to give the 1,4-dicarbonyl compound (10). The reaction is preferably carried out in the presence of a zinc (II) salt (for example, zinc chloride) in a non-polar, aprotic solvent (for example, benzene or toluene). Preferably a tertiary alcohol (for example, t-butanol) and a tertiary amine (for example, triethylamine) are also added. The reaction may be carried out at room temperature, for example over a period of 12 to 48 hours.

The 1,4-dicarbonyl compound (10) may then be reacted with aminoarene (11) to form the trisubstituted pyrroles of the present invention (1). The reaction may be carried out in a non-polar, aprotic solvent (for example dioxane). The reaction mixture may be subject to heating (for example between 150 and 170° C.) and/or microwave irradiation. The reaction may be carried out for between 1 and 12 hours, for example between 1 and 6 hours. A strong acid (e.g. p-toluenesulphonic acid) may also be added as a catalyst.

Alternatively, compounds of formula (10) where R² and R³ are both hydrogen can be prepared by the synthetic route as shown in Scheme 2.

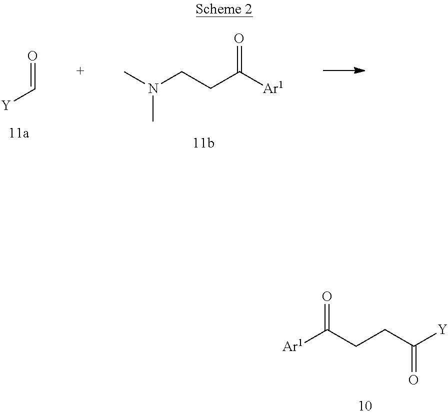

Starting aldehyde (11a) may be prepared from the corresponding acid by reduction with a reducing agent (for example NaBH₄), followed by oxidation with a suitable oxidising agent. One such example of an oxidising agent to prepare the aldehyde without further oxidation to the carboxylic acid is Dess-Martin periodinane. Starting amine (11b) may be prepared via a Mannich reaction with dimethylamine hydrochloride and formaldehyde in a polar, protic solvent (for example ethanol) in the presence of an acid catalyst.

Compounds of formula (10) can then be prepared by reacting compound (11a) and (11b) in a polar, aprotic solvent (for example, 1,2-dimethoxyethane) with a suitable catalyst. One such class of suitable catalysts are thiazolium salts (for example, 3-ethyl-5-(2-hydroxyethyl)-4-methylthiazoliumbromide). The reaction is typically carried out at elevated temperatures (for example between 80° C. and 120° C.) for between 1 and 24 hours, even more preferably between 2 and 12 hours.

Once formed, one compound of the formula (1) may be transformed into another compound of the formula (1) using standard chemistry procedures well known in the art. For examples of functional group interconversions, see for example, March's Advanced Organic Chemistry, Michael B. Smith & Jerry March, 6^{th} Edition, Wiley-Blackwell (ISBN: 0-471-72091-7), 2007 and Organic Syntheses, Volumes 1-9, John Wiley, edited by Jeremiah P. Freeman (ISBN: 0-471-12429), 1996. Compounds of the formula (1) where Y is substituted with a substituent R⁶ wherein R⁶ is an amide group of the formula C(O)NHR⁸, wherein R⁸ is an optionally substituted C₁₋₈ hydrocarbon group can be prepared by according to the synthetic route as shown in Scheme 3.

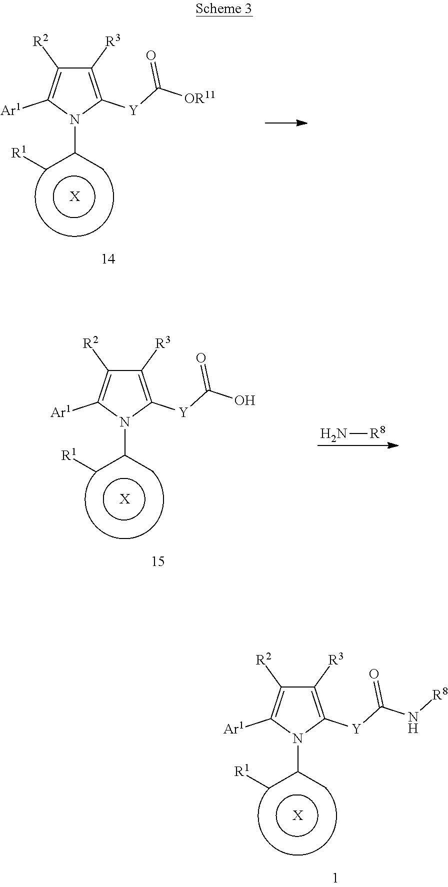

In Scheme 3, Y represents ring Y as defined herein.

A compound of the formula (14) can be prepared in accordance with the synthetic route as shown in Scheme 1 above, wherein R¹¹ is a C₁₋₈ hydrocarbon group or another carboxylic acid protecting group. Ester (14) can be hydrolysed to give carboxylic acid (15). This is preferably carried out in a mixture of a non-polar, aprotic solvent (for example, tetrahydrofuran) and a polar, protic solvent (for example, water). One such suitable solvent system is a 1:1 mixture of tetrahydrofuran and water. A strong, water-soluble base (for example, lithium hydroxide) is added and the reaction mixture is stirred at room temperature for an extended period, for example between 6 and 48 hours, more usually between 12 and 48 hours.

The acid compound (15) may then be reacted with a corresponding amine (H₂N—R) under amide-forming conditions, for example in the presence of a reagent of the type commonly used in the formation of amide bonds, to afford a compound of the formula (1) wherein R⁶ is an amide. Examples of such reagents include carbodiimide-based coupling agents such as 1,3-dicyclohexylcarbo-diimide (DCC) (Sheehan et al, J. Amer. Chem Soc. 1955, 77 1067) and 1-ethyl-3-(3′-dimethylaminopropyl)-carbodiimide (referred to herein either as EDC or EDCI) (Sheehan et al, J. Org. Chem., 1961, 26, 2525), which are typically used in combination with 1-hydroxy-7-azabenzotriazole (HOAt) (L. A. Carpino, J. Amer. Chem. Soc., 1993, 11, 4397) or 1-hydroxybenzotriazole (HOBt) (Konig et al, Chem. Ber., 103, 708, 2024-2034). Further examples of such reagents are uronium-based coupling agents such as O-(7-azabenzotriazol-1-yl)-N,N,N′,N′-tetramethyluronium hexafluorophosphate (HATU). One preferred amide coupling agent is HATU.

The coupling reaction is typically carried out in a non-aqueous, non-protic solvent such as dimethylformamide at room temperature in the presence of a non-interfering base, for example a tertiary amine such as triethylamine or N,N-diisopropylethylamine.

Compounds of formula (15) may alternatively be prepared from the hydrolysis of the corresponding nitrile, using appropriate hydrolysis conditions. Preferably the hydrolysis is carried out with a strong base, for example an alkali metal hydroxide (for example, sodium hydroxide) in a polar protic solvent or a mixture of polar protic solvents. One such example of a suitable solvent system in a mixture of methanol and water. The reaction is preferably carried out at elevated temperature for between 12 and 24 hours.

Compounds of the formula (1) where Y is substituted with a substituent R⁶ wherein R⁶ is an amine group having the formula NHR⁹ can be prepared by according to the synthetic route as shown in Scheme 4.

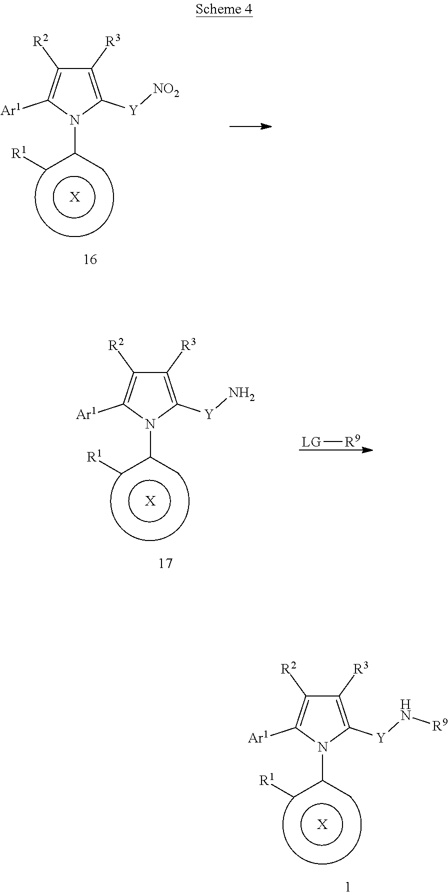

In Scheme 4, Y represents ring Y as defined herein.

A compound of formula (16) can be prepared according to the synthetic route as shown in Scheme 1 above. Compound (16) can then be reduced to compound (17) using a suitable reducing agent (for example, sodium borohydride) and optionally with catalytic quantities of a copper (II) salt (for example, copper (II) acetate). The reaction is preferably carried out in an anhydrous, polar, aprotic solvent (for example, methanol).

Compound (17) can then be reacted with a compound of the formula LG-R⁹, wherein LG is a suitable leaving group (for example, halogen, more preferably chlorine) and R⁹ is an optionally substituted non-aromatic C₁₋₈ hydrocarbon group. The amine compound (17) is first treated with a suitable base (for example, sodium hydride) in a polar, aprotic solvent (for example, dimethylformamide), typically at room temperature and is then reacted with compound LG-R⁹, typically at an elevated temperature (for example, between 60° C. and 100° C.).

Alternatively, compounds of formula (1) where R⁶ is an amide in which the nitrogen atom of the amide is bonded to ring Y can be prepared from compounds of formula (17) in an analogous method to the method shown in Scheme 4 and carboxylic acids, or activated derivatives (such as acyl chlorides or acid anhydrides).

Alternatively, the compounds of formula (1) wherein R⁶ is an amide of the formula NHCOR¹⁰ where R¹⁰ is an optionally substituted C₁₋₈ hydrocarbon group, can be prepared from intermediate (17), under amide-forming conditions, for example in the presence of a reagent of the type commonly used in the formation of amide bonds, according to Scheme 5.

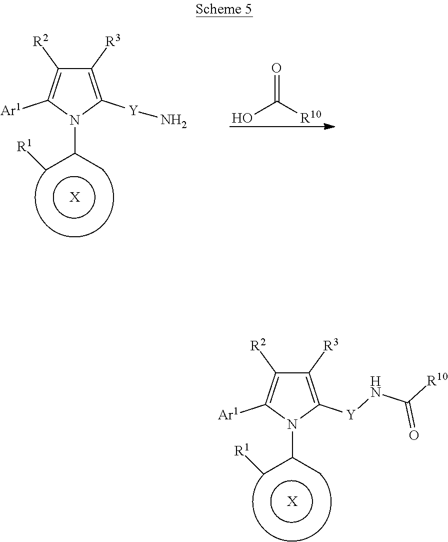

In Scheme 5, Y represents ring Y as defined herein.

Examples of such reagents include carbodiimide-based coupling agents such as 1,3-dicyclohexylcarbo-diimide (DCC) (Sheehan et al, J. Amer. Chem Soc. 1955, 77, 1067) and 1-ethyl-3-(3′-dimethylaminopropyl)-carbodiimide (referred to herein either as EDC or EDCI) (Sheehan et al, J. Org. Chem., 1961, 26, 2525), which are typically used in combination with 1-hydroxy-7-azabenzotiazole (HOAt) (L. A. Carpino, J. Amer. Chem. Soc., 1993, 11§, 4397) or 1-hydroxybenzotriazole (HOBt) (Konig et al, Chem. Ber., 103, 708, 2024-2034). Further examples of such reagents are uronium-based coupling agents such as O-(7-azabenzotriazol-1-yl)-N,N,N′,N′-tetramethyluronium hexafluorophosphate (HATU). One preferred amide coupling agent is HATU.

The coupling reaction is typically carried out in a non-aqueous, non-protic solvent such as dimethylformamide at room temperature in the presence of a non-interfering base, for example a tertiary amine such as triethylamine or N,N-diisopropylethylamine.

Compounds of the formula (1) where Y is substituted with a substituent R⁶ wherein R⁶ is an ether group having the formula OR¹² where R¹² is an optionally substituted C₁₋₈ hydrocarbon group can be prepared by according to the synthetic route as shown in Scheme 6.

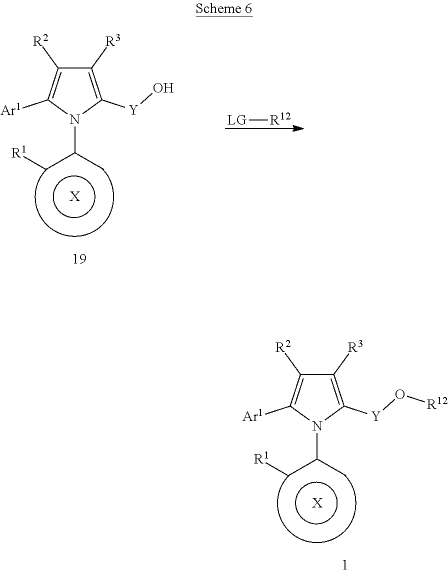

In Scheme 6, Y represents ring Y as defined herein.

A compound of formula (19) can be prepared according to the synthetic route as shown in Scheme 1 above. Compound (19) can then be reacted with a compound of the formula LG-R¹², wherein LG is a suitable leaving group (for example, halogen, more preferably chlorine) and R⁷ is an optionally substituted non-aromatic C₁₋₈ hydrocarbon group. The alcohol compound (19) is first deprotonated with a suitable base (for example, sodium hydride) in a polar, aprotic solvent (for example, dimethylformamide). This reaction may be carried out at room temperature. The reaction mixture is then treated with compound of the formula LG-R¹². The second step of this reaction may occur at elevated temperatures, typically between 80° C. and 100° C.

Compounds of formula (1) wherein R⁶ is Q¹-R^{a}—R^{b} and Q¹ is a methylene group can be prepared according to Scheme 7.

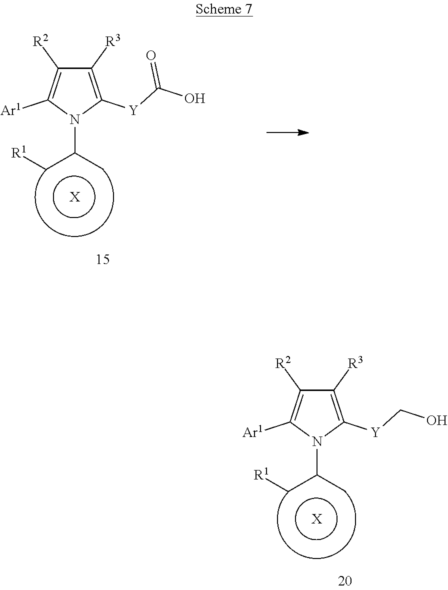

In Scheme 7, Y represents ring Y as defined herein.

Compound (15) (obtainable as described in Scheme 3 above) is treated with a reducing agent (for example sodium borohydride) in an polar aprotic solvent, such as tetrahydrofuran, to afford the primary alcohol (20). Alcohol (20) can then be reacted in the manner described above in Scheme 6 to provide further compounds of formula (1) wherein R⁶ is an ether.

Alternatively, compound (20) may undergo other standard functional group interconversions to yield further compounds of formula (1), for example via oxidation to an aldehyde and reductive amination to form an amine. Amines produced via this method can be further reacted with carboxylic acids or acid derivatives to yield amide compounds of formula (1) using the method described above in Scheme 5.

Compounds of the formula (1) wherein Z is a 1,4,5-trisubstituted pyrazole can be prepared by reacting an aryl hydrazine (21) with the α,β-unsaturated carbonyl compound (22) as shown in Scheme 8.

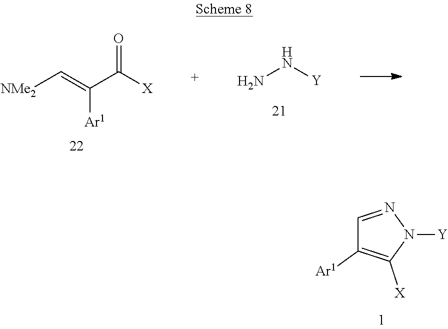

In Scheme 8, X and Y represent rings X and Y respectively as defined herein.

The aryl hydrazine (21) and α,β-unsaturated carbonyl compound (22) are dissolved in a suitable polar, protic solvent system (e.g. 1:1 water-methanol) with a suitable base (e.g. sodium carbonate). The mixture is typically stirred at or about room temperature (e.g. for about 15 minutes) before a weak acid, such as acetic acid, is added. The resulting mixture is then heated (e.g. between 100° C. and 140° C., for an extended period of time, (for example between 6 and 12 hours), for a period of time (e.g. 8 hours) sufficient to afford a compound of formula (1) wherein Z is a 1, 4, 5-trisubstituted pyrazole.

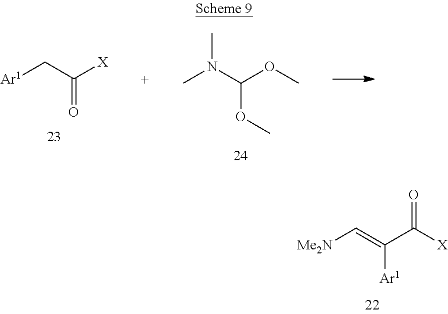

The starting α,β-unsaturated carbonyl compound (22) of Scheme 8 can be prepared from the corresponding ketone (23) and N,N-dimethylformamide dimethyl acetal. A solution of N,N-dimethylformamide dimethyl acetal in a polar aprotic solvent such as DMF, is added to a solution of ketone (23). The mixture is typically heated, for example to a temperature between 70° C. and 110° C. (e.g. approximately 90° C.) to afford compound (22). Compound (23) may be obtained through a Grignard reaction between Ar¹CH₂CHO and Br—X followed by oxidation of the resulting alcohol with a suitable oxidising agent (for example, Dess-Martin periodinane) in a solvent such as DCM to afford ketone (23).

Alternatively, when alternative isomers of formula (1) wherein Z is a 3, 4, 5-trisubstituted pyrazole, are required, these can be prepared as described in Scheme 10 below.

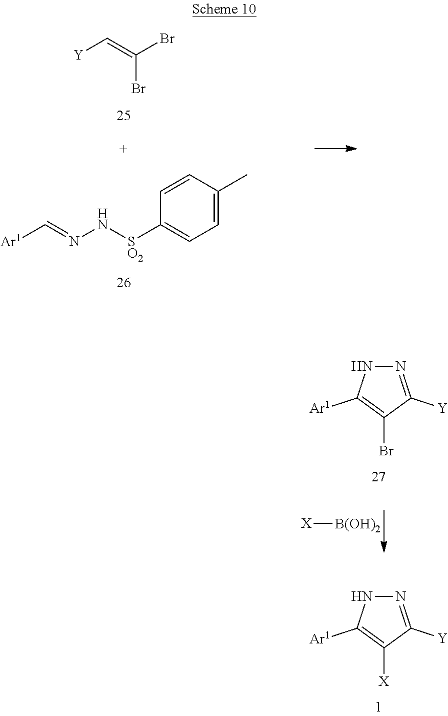

In Scheme 10, X and Y represent rings X and Y respectively as defined herein.

Alkenyl bromide (25) is reacted with diazo compound (26) in a 1,3-dipolar cycloaddition reaction by mixing the two compounds with a strong base (e.g. sodium hydroxide) and heating (e.g. to a temperature of approximately 70° C.) to afford bromo-pyrazole (27).

The bromo-pyrazole (27) is then reacted with a boronic acid having formula X—B(OH)₂ (wherein X is a ring as defined herein) in a polar solvent such as dioxane in the presence of a palladium (0) catalyst, such as bis(tri-tert-butylphosphine)palladium (0), and suitable base (such as caesium or potassium carbonate or phosphate) under Suzuki reaction conditions to give the compound of formula (1) wherein Z is a pyrazole or a protected derivative thereof. The bromo-pyrazole (27) may be in a protected form. For example, in the NH group on the pyrazole, a protecting group such as a Boc (tert-butoxycarbonyl) group may be attached to the nitrogen atom, replacing the hydrogen atom. After the reaction between the boronic acid and the pyrazole (27), a deprotection step may be required in order to give the compound of formula (1). In the case of a Boc protecting group, this can be removed by treatment with an acid such as hydrochloric acid.

Boronates and boronic acids are widely available commercially or can be prepared for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Starting material (25) can be prepared by treating the aryl aldehyde with carbon tetrabromide and triphenylphosphine in a solvent such as DCM at a reduced temperature (e.g. approximately 0° C.). Starting material (26) can be prepared from the corresponding aryl aldehyde by treating with p-toluenesulfonyl hydrazide in a polar protic solvent such as methanol and heating (e.g. to approximately 60° C.).

Compounds of formula (1) wherein Z is an isoxazole group may be prepared according to the synthetic scheme in Scheme 11.

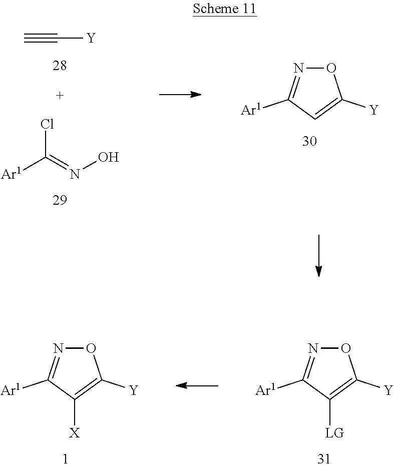

In Scheme 11, X and Y represent rings X and Y respectively as defined herein.

Intermediate (30) can be prepared by reacting alkyne (28) with oxime (29) by mixing in a polar, aprotic solvent (such as diethyl ether) with a base (such as triethylamine), for example at a temperature around room temperature to afford isoxazole (30). Isoxazole (30) can then be brominated, with a suitable brominating agent, such as N-bromosuccinimide as a bromine source, to afford the bromoisoxazole (31). The reaction typically takes place in an acidic solution (e.g. acetic acid) at elevated temperatures (for example between 90° C. and 120° C.).

The bromo-isoxazole (31) is then reacted with a boronic acid having formula X—B(OH)₂ (wherein X is a ring as defined herein) in a polar solvent such as dioxane in the presence of a palladium (0) catalyst, such as bis(tri-tert-butylphosphine)palladium (0), and a base (e.g. caesium or potassium carbonate or phosphate) under Suzuki reaction conditions to give the compound of formula (1) wherein Z is a isoxazole or a protected derivative thereof. The bromo-isoxazole (31) may be in a protected form. For example, in a NH group on groups Ar₁ or Y, a protecting group such as a Boc (tert-butoxycarbonyl) group may be attached to the nitrogen atom, replacing the hydrogen atom. After the reaction between the boronic acid and the isoxazole (31), a deprotection step may be required in order to give the compound of formula (1). In the case of a Boc protecting group, this can be removed by treatment with an acid such as hydrochloric acid.

Boronates and boronic acids are widely available commercially or can be prepared for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Starting material (29) can be prepared from the corresponding aryl aldehyde via a two-step process. The first step consists of treating the aldehyde with NH₂OH and a strong base (such as sodium hydroxide) in a polar, protic solvent system (such as 1:1 ethanol:water) to afford the aryl oxime. This can then the chlorinated by mixing with N-chlorosuccinimide in dimethylformamide and stirring for 18 hours to afford starting material (29).

The synthesis of the compounds of formula (1) has been illustrated above with reaction schemes for preparing pyrroles, isoxazoles and pyrazoles. It will readily be appreciated however that analogous methods may be used to prepare compounds of formula (1) containing other five-membered heteroaryl rings.

Specific synthetic routes for the preparation of a preferred atropisomer, compound (1), of the invention are shown in Scheme 12 below.

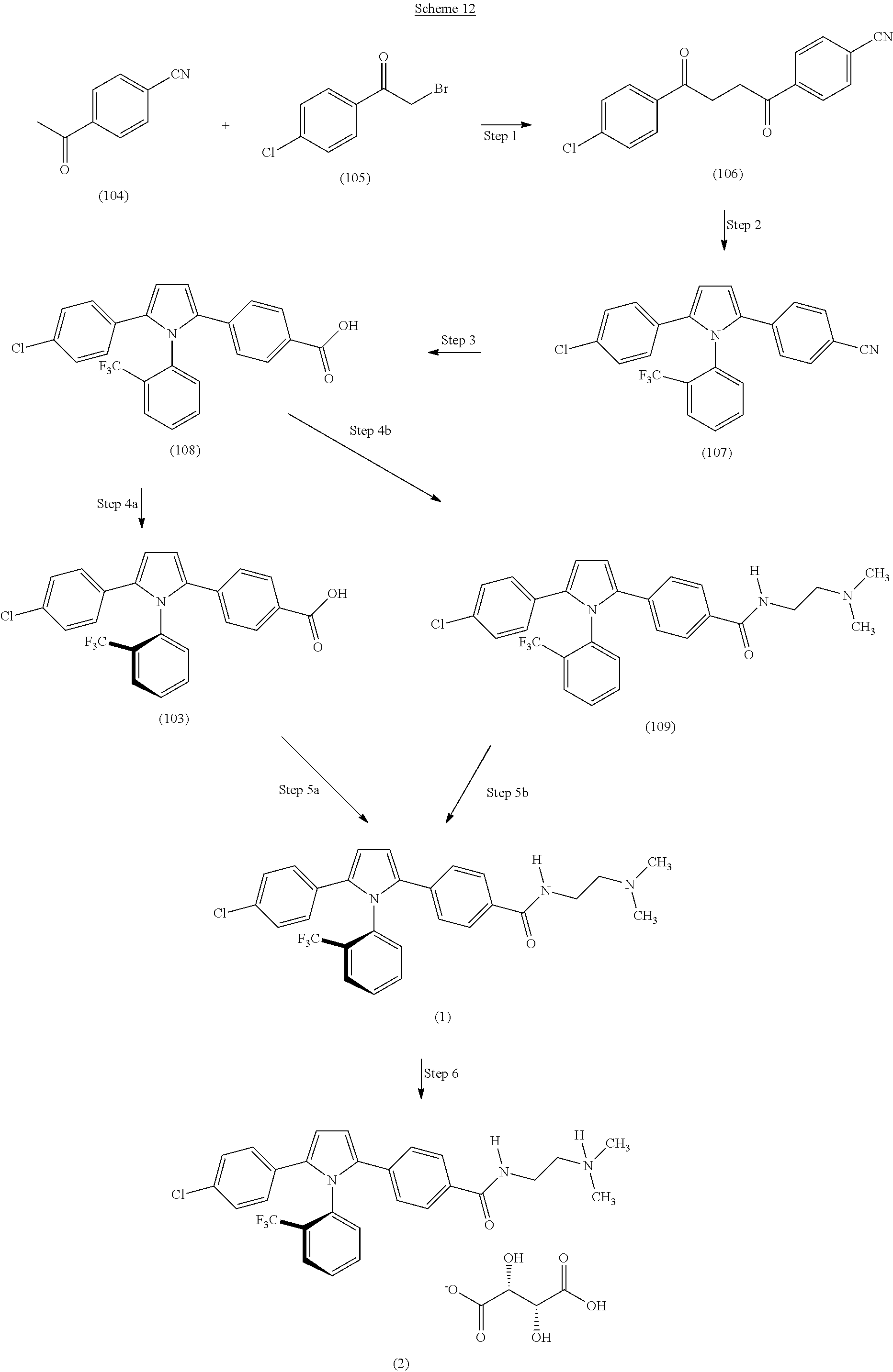

The starting materials for the synthetic route shown in Scheme 1 are 4-cyano-acetophenone (104) and 4-chlorophenacylbromide (105), both of which are commercially available.

In Step 1, 4-cyano-acetophenone (104) and 4-chlorophenacylbromide (105) are reacted together to give 4-[4-(4-chlorophenyl)-4-oxo-butanoyl]benzonitrile (106). The reaction is typically carried out in the presence of a zinc (II) salt (for example, zinc chloride) in a suitable solvent, for example a mixture of a non-polar (e.g. hydrocarbon) solvent such as benzene or toluene and a tertiary alcohol (for example, t-butanol), in the presence of a tertiary amine such as triethylamine. The reaction may be carried out at room temperature, or near room temperature, for example over a period of 12 to 60 hours.

In Step 2, 4-[4-(4-chlorophenyl)-4-oxo-butanoyl]benzonitrile (106) is reacted with 2-trifluoromethyl aniline to give 4-(5-(4-chlorophenyl)-1-(2-(trifluoromethyl) phenyl)-1H-pyrrol-2-yl) benzonitrile (107). The reaction is typically carried out in the presence of an acid catalyst such as p-toluenesulphonic acid in a suitable high boiling solvent (for example dioxane) at an elevated temperature (for example between 130 and 170° C.) and/or microwave irradiation. The reaction may be carried out for between 1 and 12 hours, for example between 1 and 6 hours.

In Step 3, 4-(5-(4-chlorophenyl)-1-(2-(trifluoromethyl) phenyl)-1H-pyrrol-2-yl) benzonitrile (107) is subjected to alkaline hydrolysis to give 4-(5-(4-chlorophenyl)-1-(2-(trifluoromethyl) phenyl)-1H-pyrrol-2-yl) benzoic acid (108). The hydrolysis reaction is typically carried out in an aqueous solvent, which may contain an alcohol such as methanol, in the presence of an alkaline metal hydroxide such as sodium hydroxide (typically in an excess amount), and generally with heating, for example to a temperature in the range from 60-80° C. or a period of up to about 20 hours, or more. Once hydrolysis is complete, the acid (8) is typically isolated by cooling and acidifying the reaction mixture.

Following Step 3, one of two possible routes to the atropisomer (1) can be followed. In one variant consisting of Steps 4b and 5b and 6, 4-(5-(4-chlorophenyl)-1-(2-(trifluoromethyl) phenyl)-1H-pyrrol-2-yl) benzoic acid (108) is reacted with N,N-dimethylethylenediamine under amide forming conditions to give a racemic mixture of atropisomers of 4-(5-(4-chlorophenyl)-1-(2-(trifluoromethyl) phenyl)-1H-pyrrol-2-yl)-N-(2-(dimethylamino) ethyl) benzamide (109) which is then resolved into its individual atropisomers by chiral separation to give the atropisomer (1).

In the other variant, racemic 6, 4-(5-(4-chlorophenyl)-1-(2-(trifluoromethyl) phenyl)-1H-pyrrol-2-yl) benzoic acid (108) is subjected to a chiral separation to give the atropisomer (103) which is then reacted with N,N-dimethylethylenediamine under amide forming conditions to give atropisomer (1).

The carboxylic acids (103) and (108) are reacted with N,N-dimethylethylenediamine under amide forming conditions in the presence of an amide coupling reagent. Examples of such amide coupling reagents include carbodiimide-based coupling reagents such as 1,3-dicyclohexylcarbo-diimide (DCC) (Sheehan et al, J. Amer. Chem Soc. 1955, 77, 1067) and 1-ethyl-3-(3′-dimethylaminopropyl)-carbodiimide (referred to herein either as EDC or EDCI) (Sheehan et al, J. Org. Chem., 1961, 26, 2525), which are typically used in combination with 1-hydroxy-7-azabenzotriazole (HOAt) (L. A. Carpino, J. Amer. Chem. Soc., 1993, jj, 4397) or 1-hydroxybenzotriazole (HOBt) (Konig et al, Chem. Ber., 103, 708, 2024-2034), uronium-based coupling reagents such as 0-(7-azabenzotriazol-1-yl)-N,N,N′,N′-tetramethyluronium hexafluorophosphate (HATU), and propanephosphonic acid anhydride (T3P) (see A. Garcia, Synlett, 2007, No. 8, pp 1328-1329). Particular amide coupling reagents for use in process steps 5a and 5b are HATU and T3P.

The amide coupling reaction is typically carried out in a non-aqueous, polar, non-protic solvent such as tetrahydrofuran or dimethylformamide, or mixtures thereof at room temperature or thereabouts (e.g. 18-30° C.) in the presence of a non-interfering base, for example a tertiary amine such as triethylamine or N,N-diisopropylethylamine.

Certain aspects of the processes described above represent further embodiments of the invention (Embodiments 2.1 to 2.8). Accordingly, the invention provides:
2.1 A method of preparing a composition of matter or a single atropisomer as defined in any one of Embodiments 1.1 to 1.211, which method comprises the chiral separation of mixture of atropisomers of a compound of the formula (0):

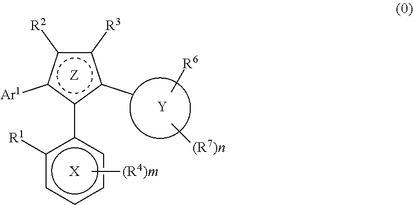

where ring X, ring Y, ring Z, Ar¹, m, n and R¹ to R⁷ are as defined in any one of Embodiments 1.1 to 1.211.
2.2 A method according to Embodiment 2.1 wherein the mixture of atropisomers of the compound of formula (0) is a racemic mixture.
2.3 A method according to Embodiment 2.1 or Embodiment 2.2 wherein the chiral separation is carried out by:
   (i) passing the mixture of atropisomers through a chiral chromatography column; e.g. a chiral HPLC column; or
   (ii) reacting the mixture of atropisomers of a compound of the formula (0) with a chiral acid to form salts of both of the atropisomers in the mixture, separating the salts and decomposing the salts to give the corresponding free bases of each of the atropisomers.
2.4 A method for the preparation of atropisomer (1) as defined herein, which method comprises the reaction of a compound of the formula (103) with N,N-dimethylethylenediamine under amide forming conditions.
2.5 A method according to Embodiment 2.4 wherein the amide forming conditions include the presence of an amide coupling reagent, for example an amide coupling agent as described herein.
2.6 A method according to Embodiment 2.5 wherein the amide coupling reagent is propanephosphonic acid anhydride (T3P).
2.7 A method for the preparation of a compound of the formula (103) (see Scheme 12), which method comprises the chiral separation of the compound of formula (103) from a mixture of atropisomers of formula (108), for example by chiral chromatography or salt formation with a chiral base and resolution of the resulting chiral salt.
2.8 An atropisomer compound having the formula (103), or a salt thereof (for example a metal salt such as an alkaline or alkaline earth metal salt, or a salt with ammonia or an organic amine).

The atropisomers and compositions of matter of the invention can be provided in salt forms or in non-salt (e.g. free base) form.

Acid addition salts of basic atropisomers of the invention can be prepared by bringing an atropisomer in free base form into contact with a suitable salt forming acid in a suitable solvent or mixture of solvents as described elsewhere herein and then isolating the desired salt from the solvent or mixture of solvents.

A particular salt of the invention is the (+)-L-tartaric acid salt of formula (2) as defined in any one of Embodiments 1.194 to 1.211.

The (+)-L-tartaric acid salt of the invention can be prepared from the atropisomer of the formula (1) by reaction with tartaric acid in a solvent or mixture of solvents and then isolating the tartrate salt from the solvent or mixture of solvents.

In one embodiment (Embodiment 2.9), the atropisomer of formula (1) can be dissolved or suspended in one solvent to form a first mixture, and (+)-L-tartaric acid dissolved or suspended in the same or another solvent to form a second mixture, and then the first and second mixtures combined and left (e.g. with stirring) for a period of time to allow salt formation to occur, followed by isolation of the (+)-L-tartaric acid salt.

When the first and second mixtures are combined, it is preferred that the molar amounts of atropisomer of formula (1) and (+)-L-tartaic acid are approximately equivalent; i.e. there is preferably a 1:1 molar ratio between the atropisomer of formula (1) and (+)-L-tartaic acid.

The (+)-L-tartaic acid salt can be isolated from the combined mixture by filtration (when a precipitate is formed) or by evaporation of the solvents.

Thus, when more than one solvent is present in the combined mixture, the different solvents can be selected so as to act as co-solvents or as anti-solvents.

The solvent or mixture of solvents can be selected so that they retain the (+)-L-tartaric acid salt at least partially in solution when heated, but then deposit the salt as a precipitate when the solvent or mixture of solvents is cooled.

The solvent used to form the first mixture (the mixture containing the atropisomer of formula (1)) can be selected from, for example, aliphatic ketones, aliphatic esters of aliphatic acids, non-aromatic cyclic ethers and aliphatic alcohols.

A particular example of an aliphatic ketone is acetone.

Examples of aliphatic esters of aliphatic acids include C₂₋₄ alkyl esters of acetic acid, a particular example being isopropylacetate.

Examples of non-aromatic cyclic ethers include dioxane, 2-methyltetrahydrofuran and tetrahydrofuran, a particular example being 2-methyltetrahydrofuran.

Examples of aliphatic alcohols are C₂₋₄ aliphatic alcohols, and more particularly C₃₋₄ alkanols such as isopropyl alcohol and butanol.

The solvent used to form the second mixture (the mixture containing the (+)-L-tartaric acid) can be selected from, for example, water, non-aromatic cyclic ethers and aliphatic alcohols.

A particular example of an aliphatic alcohol solvent for the second mixture is ethanol.

A particular example of a non-aromatic cyclic ether solvent for the second mixture is tetrahydrofuran (THF).

Another particular example of a solvent for use in forming the second mixture is water.

The (+)-L-tartaric acid salt of the atropisomer of formula (1) can exist in several crystalline forms, notably Pattern A (which is a solvate) and Pattern B (which is an anhydrate). Characterising details for the different crystalline forms are provided elsewhere herein. The different crystalline forms can be prepared by varying the solvents and heating conditions used in the formation of the salts.

In one process (Embodiment 2.10) for making (+)-L-tartaric acid salt of the atropisomer of formula (1) having Pattern A, a solution of the atropisomer in acetone is mixed with a solution of (+)-L-tartaric acid in ethanol at a temperature in the range from 20° C. to 30° C. (for example approximately 25° C.), the resulting mixture is stirred or otherwise agitated for a length of time (e.g. 12-24 hours) sufficient to allow salt formation to take place, and the salt is then isolated by filtration.

In another process (Embodiment 2.11) for making (+)-L-tartaric acid salt of the atropisomer of formula (1) having Pattern A, a solution of the atropisomer in in isopropyl alcohol is mixed with a solution of (+)-L-tartaric acid in ethanol at a temperature in the range from 35° C. to 45° C. (for example approximately 40° C.), the resulting mixture is cooled to a temperature in the range from 20° C. to 30° C. (for example approximately 25° C.) over a period of approximately 1-3 hours, and the salt is then isolated by filtration.

In another process (Embodiment 2.12) for making (+)-L-tartaric acid salt of the atropisomer of formula (1) having Pattern A, a solution of the atropisomer in 2-methyltetrahydrofuran is mixed with a solution of (+)-L-tartaric acid in ethanol at a temperature in the range from 20° C. to 30° C. (for example approximately 25° C.), the resulting mixture is stirred or otherwise agitated for a length of time (e.g. 12-24 hours) sufficient to allow salt formation to take place, and the salt is then isolated by filtration.

In one process (Embodiment 2.13) for making (+)-L-tartaric acid salt of the atropisomer of formula (1) having Pattern B, a solution of the atropisomer in isopropyl acetate at a temperature in the range from 35° C. to 45° C. (for example approximately 40° C.) is mixed with a solution of (+)-L-tartaric acid in ethanol, the resulting mixture is cooled to a temperature in the range from 20° C. to 30° C. (for example approximately 25° C.) over a period of approximately 1-3 hours, and the salt is then isolated by filtration.

In another process (Embodiment 2.14) for (+)-L-tartaric acid salt of the atropisomer of formula (1) having Pattern B, a solution of the atropisomer in isopropyl acetate at a temperature in the range from 35° C. to 45° C. (for example approximately 40° C.) is mixed (either portion-wise or in one single charge) with a solution of (+)-L-tartaric acid in THF and one or more seed crystals of the salt Pattern B are added to give a precipitate, the mixture is cooled to a temperature in the range from 20° C. to 30° C. (for example approximately 25° C.) and stirred or agitated for period of time (e.g. 12 to 24 hours, particularly approximately 20 hours) sufficient to allow ripening of the precipitate to a state in which it can be isolated by filtration.

In another process (Embodiment 2.15) for (+)-L-tartaric acid salt of the atropisomer of formula (1) having Pattern B, a solution of the atropisomer in butanol at a high temperature in the range from 70° C. to 85° C. (for example approximately 80° C.) is mixed (either portion-wise or in one single charge) with a solution of (+)-L-tartaric acid in water, the resulting mixture is cooled to an intermediate temperature in the range 65° C. to 70° C. before adding one or more seed crystals of the salt Pattern B and cooling the mixture to a low temperature in the range from 3-10° C. over a period of 8 to 15 hours, and thereafter stirring or otherwise agitating the resulting mixture at or near the low temperature for a further period of 2 to 8 hours (e.g. approximately 6 hours) and then filtering off the Pattern B salt thus formed.

Protecting Groups

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a tetrahydropyranyl (THP) ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol.

The aldehyde or ketone group is readily regenerated by hydrolysis using a large excess of water in the presence of acid.

An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz or NH—Z); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH—Psec).

For example, in Scheme 1 above, when the moiety R³ in the amine H₂N—Y—R³ contains a second amino group, such as a cyclic amino group (e.g. a piperidine or pyrrolidine group), the second amino group can be protected by means of a protecting group as hereinbefore defined, one preferred group being the tert-butyloxycarbonyl (Boc) group. Where no subsequent modification of the second amino group is required, the protecting group can be carried through the reaction sequence to give an N-protected form of a compound of the formula (1) which can then be de-protected by standard methods (e.g. treatment with acid in the case of the Boc group) to give the compound of formula (1).

Other protecting groups for amines, such as cyclic amines and heterocyclic N—H groups, include toluenesulphonyl (tosyl) and methanesulphonyl (mesyl) groups, benzyl groups such as a para-methoxybenzyl (PMB) group and tetrahydropyranyl (THP) groups.

A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇alkylsilyl-C₁₋₇alkyl ester; or a C₅₋₂₀ aryl-C₁₋₇alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

Isolation and Purification of the Compounds of the Invention

The compounds prepared by the foregoing synthetic routes can be isolated and partially purified according to standard techniques well known to the person skilled in the art, to give mixtures of atropisomers. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

An example of such a system for purifying compounds via preparative LC-MS is described below in the Examples section of this application (under the heading “Mass Directed Purification LC-MS System”). However, it will be appreciated that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described below could alternatively be used to purify the compounds.

Once the mixtures of atropisomers have been isolated and purified to an acceptable extent, the mixtures can then be subjected to separation procedures in order to separate individual atropisomers. Thus, for example, chiral chromatography can be used to separate individual atropisomers. The retention times of the atropisomers in the chiral chromatography procedures provide a means of differentiating between and characterising the individual atropisomers whose NMR and MS properties are typically the same.

Chiral chromatography columns that can be used to separate the individual atropisomers comprise an immobilised chiral stationary phase (CSF) which can be, for example, based on a functionalised amylose or cellulose. Examples of such CSF's are amylose and celluloses that have been functionalised with chloro- and/or methyl-substituted phenyl carbamates. Particular examples of chiral columns that may be used to isolate the individual atropisomers of the present invention are the “Chiralpak IG” columns available from Daicel Corporation.

Mobile phases that can typically be used in conjunction with the above chiral columns include mixtures of (A) liquid alkanes such as n-heptane containing a small amount (e.g. up 1% (v/v) and more usually about 0.1% (v/v)) of an alkylamine base such as diethylamine; and (B) alcohols and mixtures thereof such as mixtures of isopropyl alcohol and methanol (e.g. 70:30 IPA:MeOH). For example, the mobile phase can comprise a mixture of A:B in the range of ratios 80:20 to 95:5, for example from approximately 85:15 to approximately 90:10. The mobile phases may be used in isocratic or gradient elution methods but, in one embodiment of the invention, are used in an isocratic elution method.

The atropisomers of the invention may also be resolved by chiral HPLC under supercritical fluid chromatography (SFC) conditions. In supercritical fluid chromatography, the mobile phase comprises a supercritical fluid such as carbon dioxide, often with a co-solvent such as an alcohol or mixture of alcohols, e.g. methanol, ethanol and isopropanol.

The Chiralpak IG columns referred to above may be used in SFC chromatography procedures, using carbon dioxide/methanol/isopropanol mixtures as the mobile phase.

Other chiral column/co-solvent combinations for use in SFC include:

### Lux Cellulose 4 (MeOH, EtOH);

### Lux Cellulose 2 (MeOH);

### Lux Amylose 1 (MeOH, EtOH); and

### YMC Amylose-SA (MeOH, EtOH)

The Lux family of chiral columns are available from Phenomenex, Inc.

YMC Amylose-SA columns are available from YMC America, Inc.

Biological Properties and Therapeutic Uses

The evidence set out in the Examples below indicates that atropisomers of the invention as defined herein are inhibitors of the polo box domains of PLK1 and PLK4 kinases but do not inhibit the catalytic domains of PLK1 and PLK4 kinases. Since PBD domains only reside in PLKs, the atropisomers should exhibit much greater selectivity (and hence fewer unwanted side effects due to off-target kinase inhibition) than compounds which are ATP-competitive kinase inhibitors. For example, the results obtained from the study described in Example 11F below, where the atropisomer of formula (1) was tested against a panel of ninety seven kinases and showed negligible activity against other kinases, confirms that the atropisomer of formula (1) has a high degree of selectivity for PLK1-PBD and PLK4-PBD over other structurally and functionally similar kinases. On the basis of this evidence, it is considered that other atropisomers of the invention, particularly those having the same R configuration as atropisomer (1), should exhibit similar advantages.

A further advantage of inhibiting the PBD domain rather than the catalytic domain is that this may result in a reduced tendency to induce drug resistance compared to PLK1 inhibitors that inhibit the catalytic domain.

The activity of the atropisomers of the invention as inhibitors of the PBD domain of PLK1 kinase can be demonstrated using the fluorescence polarization (FP) assay described in Narvaez et al., Cell Chemical Biology, 24, 1017-1028, 2017, see page 1018 and page 1026 (Method Details).

It is believed that compounds of the invention may be effective in exploiting weaknesses in cellular pathways as a result of constitutively activating KRAS mutants and therefore the composition of matter or atropisomers of the invention may be useful for the treatment of diseases and conditions mediated by modulation of KRAS.

Mutation of KRAS, resulting from a single nucleotide substitution, has been associated with various forms of cancer. In particular, KRAS mutations are found at high rates in leukaemias, colon cancer, pancreatic cancer and lung cancer.

A primary screen for anticancer activity, which makes use of a cancer cell line (U87MG, human brain (glioblastoma astrocytoma)), is described in Example 11A below.

In addition, it is believed that compounds of the invention may be useful in treating cancers characterised by p53 deficiency or mutation in the TP53 gene. PLK1 is believed to inhibit p53 in cancer cells. Therefore, upon treatment with PLK1 inhibitors, p53 in tumour cells should be activated and hence should induce apoptosis.

The activity of the composition of matter or atropisomers against KRAS mutant and p53 deficient cancers is believed to arise, at least in part, through inhibition of PLK1 kinase and, in particular, the C-terminal polo box domain (PBD) of PLK1 kinase. KRAS is known to be dependent on interaction with PLK1.

Compounds of the invention that only inhibit the PBD domain and not the N-terminal catalytic domain of PLK1 are advantageous in that they are selective for PLK1-PBD over other structurally and functionally similar kinases, against which they show negligible inhibitory activity (see Example E below).

The compositions of matter or atropisomers of the invention induce mitotic arrest with non-congressed chromosomes, a property which is believed to arise from the PLK1-PBD and PLK4-PBD inhibiting activity of the composition of matter or atropisomers (see Example 11C below).

The atropisomers induce mitotic arrest with a multipolar spindle phenotype, and causes amplification of centrioles, a well described phenotype of PLK4 inhibition (Lei 2018, Cell Death & Disease 9, 1066; Kawakami, PNAS 2018, 115(8) 1913-18). These phenotypes are believed to arise from the PLK4-PBD inhibiting activity of the atropisomers.

A further advantage of inhibiting the PBD domain rather than the catalytic domain is that this may result in a reduced tendency to induce drug resistance compared to PLK1 inhibitors that inhibit the catalytic domain.

The activity of compounds of the invention as inhibitors of the PBD domain of PLK1 kinase can be demonstrated using the fluorescence polarization (FP) assay described in Narvaez et al., Cell Chemical Biology, 24, 1017-1028, 2017, see page 1018 and page 1026 (Method Details).

Compounds of the invention have good oral bioavailability (see Example 11G below) and have good brain exposure when administered orally (see Example 11G below). Accordingly, the composition of matter or atropisomers of the invention should be useful in treating brain cancers such as gliomas and glioblastomas.

In further embodiments (Embodiments 3.1 to 3.27), the invention provides:
3.1. A composition of matter, atropisomer or salt as defined in any one of Embodiments 1.1 to 1.211 for use as a PLK1-PBD inhibitor.
3.2 A composition of matter, atropisomer or salt as defined in any one of Embodiments 1.1 to 1.211 for use as a PLK4-PBD inhibitor.
3.3 A composition of matter, atropisomer or salt as defined in any one of Embodiments 1.1 to 1.211 for use as a PLK1-PBD and PLK4-PBD inhibitor.
3.4 A composition of matter, atropisomer or salt as defined in any one of Embodiments 1.1 to 1.211 for use in treating a cancer, optionally in combination with another therapeutic agent or treatment (e.g. an anticancer agent or therapy), where the cancer is selected from tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the oesophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukaemias, lymphomas) and premalignant haematological disorders and disorders of borderline malignancy including haematological malignancies and related conditions of lymphoid lineage (for example acute lymphocytic leukaemia [ALL], chronic lymphocytic leukaemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenous leukaemia [AML], chronic myelogenous leukaemia [CML], chronic myelomonocytic leukaemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocytic leukaemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcoma protuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).
3.5 A composition of matter, atropisomer or salt as defined in any one of Embodiments 1.1 to 1.211 for use in treating a cancer, optionally in combination with another therapeutic agent or treatment (e.g. an anticancer agent or therapy), where the cancer is selected from pancreatic cancer, cancers of the large intestine and colorectum, lung cancers, cancers of the brain and nerves, and blood cancers such as lymphoma and leukaemia.
3.6 A composition of matter, atropisomer or salt as defined in any one of Embodiments 1.1 to 1.211 for use in treating a cancer, optionally in combination with another therapeutic agent or treatment (e.g. an anticancer agent or therapy), where the cancer is selected from gliomas and glioblastomas (which may, for example, be selected from glioblastoma multiforme, ependymomas, diffuse intrinsic pontine glioma, IDH1 mutant gliomas).
3.7 A composition of matter, atropisomer or salt as defined in any one of Embodiments 1.1 to 1.211 for use in treating a cancer, optionally in combination with another therapeutic agent or treatment (e.g. an anticancer agent or therapy), where the cancer is selected from rhabdoid tumours; medulloblastoma and other embryonal tumours of the brain; breast, lung, melanoma, gastric, colorectal, pancreatic and ovarian cancer.
3.8 A composition of matter, atropisomer or salt as defined in any one of Embodiments 1.1 to 1.211 for use in treating a cancer, optionally in combination with another therapeutic agent or treatment (e.g. an anticancer agent or therapy), wherein the cancer is one in which PLK1 is implicated (e.g. wherein PLK1 is overexpressed).
3.9 A composition of matter, atropisomer or salt for use according to Embodiment 3.8 wherein the cancer is as defined in any one of Embodiments 3.4 to 3.7.
3.10 A composition of matter, atropisomer or salt as defined in any one of Embodiments 1.1 to 1.211 for use in treating a cancer, optionally in combination with another therapeutic agent or treatment (e.g. an anticancer agent or therapy), wherein the cancer is one in which PLK4 is implicated (e.g. wherein PLK4 is overexpressed).
3.11 A composition of matter, atropisomer or salt for use according to Embodiment 3.10 wherein the cancer is as defined in any one of Embodiments 3.4 to 3.7.
3.12 A composition of matter, atropisomer or salt as defined in any one of Embodiments 1.1 to 1.211 for use in treating a cancer, optionally in combination with another therapeutic agent or treatment (e.g. an anticancer agent or therapy), wherein the cancer is one which is characterised by p53 deficiency or mutation in the TP53 gene.
3.13 A composition of matter, atropisomer or salt for use according to Embodiment 3.12 wherein the cancer is as defined in any one of Embodiments 3.4 to 3.7.
3.14 A composition of matter, atropisomer or salt as defined in any one of Embodiments 1.1 to 1.211 for use in treating a cancer wherein the cancer is one which is characterised by the presence of a mutated form of KRAS.
3.15 A composition of matter, atropisomer or salt for use according to Embodiment 3.14 wherein the mutated form of KRAS in one having a mutation at an amino acid in the protein selected from glycine 12, glycine 13, glutamine 61, and combinations thereof.
3.16 A composition of matter, atropisomer or salt for use according to Embodiment 3.14 or 3.15 wherein the cancer is as defined in any one of Embodiments 3.4 to 3.7.
3.17 A composition of matter, atropisomer or salt as defined in any one of Embodiments 1.1 to 1.211 for use in medicine or therapy, optionally in combination with another therapeutic agent or treatment (e.g. an anticancer agent or therapy).
3.18 A composition of matter, atropisomer or salt as defined in any one of Embodiments 1.1 to 1.211 for use, optionally in combination with another therapeutic agent or treatment (e.g. an anticancer agent or therapy), in preventing or treating disease states and conditions characterised by abnormal expression of KRAS protein.
3.19 A composition of matter, atropisomer or salt as defined in any one of Embodiments 1.1 to 1.211 for use as an anti-cancer agent.
3.20 A method of treating a subject (e.g. a mammalian subject such as human) suffering from a cancer as defined in any one of Embodiments 3.4 to 3.16, which method comprises administering to the subject a therapeutically effective amount of a composition of matter, atropisomer or salt as defined in any one of Embodiments 1.1 to 1.211, optionally in combination with another therapeutic agent or treatment (e.g. an anticancer agent or therapy).
3.21 The use of a composition of matter, atropisomer or salt as defined in any one of Embodiments 1.1 to 1.211 for the manufacture of a medicament for a use as defined in any one of Embodiments 3.1 to 3.19.
3.22 A method of inhibiting PLK1-PBD, which method comprises bringing an effective kinase inhibiting amount of a composition of matter, atropisomer or salt as defined in any one of Embodiments 1.1 to 1.211 into contact with the PLK1-PBD.
3.23 A method of inhibiting PLK1 kinase, which method comprises contacting the PLK1 kinase with a kinase inhibiting amount of a composition of matter, atropisomer or salt as defined in any one of Embodiments 1.1 to 1.211.
3.24 A method of inhibiting PLK4-PBD, which method comprises bringing an effective inhibiting amount of a composition of matter, atropisomer or salt as defined in any one of Embodiments 1.1 to 1.211 into contact with the PLK4-PBD.
3.25 A method of inhibiting PLK4 kinase, which method comprises contacting the PLK4 kinase with a kinase inhibiting amount of a composition of matter, atropisomer or salt as defined in any one of Embodiments 1.1 to 1.211.
3.26 A method of inhibiting PLK1-PBD and PLK4-PBD, which method comprises bringing an effective inhibiting amount of a composition of matter, atropisomer or salt as defined in any one of Embodiments 1.1 to 1.211 into contact with the PLK1-PBD and PLK4-PBD.
3.27 A method according to any one of Embodiments 3.22 to 3.26 wherein the effective inhibiting amount of a composition of matter, atropisomer or salt as defined in any one of Embodiments 1.1 to 1.211 is brought into contact with the PLK1-PBD and/or PLK4-PBD in vivo, for example in a mammalian subject such as a human subject.

Prior to administration of a composition of matter, atropisomer or salt as defined in any one of Embodiments 1.1 to 1.211, a patient may be screened to determine whether a cancer from which the patient is or may be suffering is one which is characterised by elevated levels of PLK1 and/or PLK4 kinase and which would therefore be would be susceptible to treatment with a compound having activity against PLK1 and/or PLK4 kinase.

For example, a biological sample taken from a patient may be analysed to determine whether a cancer, that the patient is or may be suffering from is one which is characterised by a genetic abnormality or abnormal protein expression which leads to up-regulation of PLK1 and/or PLK4 kinase. The term up-regulation includes elevated expression or over-expression, including gene amplification (i.e. multiple gene copies) and increased expression by a transcriptional effect, and hyperactivity and activation, including activation by mutations. Thus, the patient may be subjected to a diagnostic test to detect a marker characteristic of up-regulation of PLK1 and/or PLK4 kinase. The term diagnosis includes screening. By marker we include genetic markers including, for example, the measurement of DNA composition to identify mutations of PLK1 and/or PLK4 kinase. The term marker also includes markers which are characteristic of up-regulation of PLK1 and/or PLK4, including enzyme activity, enzyme levels, enzyme state (e.g. phosphorylated or not) and mRNA levels of the aforementioned proteins.

Tumours with upregulation of PLK1 and/or PLK4 kinase may be particularly sensitive to PLK1 inhibitors. Tumours may preferentially be screened for upregulation of PLK1 and/or PLK4. Thus, the patient may be subjected to a diagnostic test to detect a marker characteristic of up-regulation of PLK1 and/or PLK4. The diagnostic tests are typically conducted on a biological sample selected from tumour biopsy samples, blood samples (isolation and enrichment of shed tumour cells), stool biopsies, sputum, chromosome analysis, pleural fluid and peritoneal fluid.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively, a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) pre-hybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labelled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of up-regulation of PLK1 and/or PLK4 kinase could be applicable in the present case.

Alternatively, or in addition, prior to administration of a composition of matter, atropisomer or salt as defined in any one of Embodiments 1.1 to 1.211, a patient may be screened to determine whether a cancer from which the patient is or may be suffering is one which is characterised by mutated KRAS and which would therefore be would be susceptible to treatment with a compound having activity against cancer cells carrying a mutant KRAS.

For example, a biological sample taken from a patient may be analysed to determine whether a cancer, that the patient is or may be suffering from is one which is characterised by a presence of mutant KRAS. Thus, for example, the patient may be subjected to a diagnostic test to detect mutations in at codons 12, 13, 61 (glycine 12, glycine 13 and glutamine 61) or mixtures thereof in the KRAS protein. Commercially available diagnostic tests for mutant KRAS include the Cobas® KRAS Mutation Test from Roche Molecular Systems, Inc and therascreen KRAS RGQ PCR Kit from Qiagen Manchester, Ltd.

Tumours with mutant KRAS may be particularly sensitive to PLK1 and/or PLK4 inhibitors. Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation as described above.

Accordingly, in further embodiments (Embodiments 3.28 to 3.38), the invention provides:
3.28 A composition of matter, atropisomer or salt as defined in any one of Embodiments 1.1 to 1.211 for use in the treatment of a cancer in a subject (e.g. a human subject) who has been screened and has been determined as suffering from a cancer which is characterised by elevated levels of PLK1 kinase (e.g. PLK1 overexpression).
3.29 A composition of matter, atropisomer or salt as defined in any one of Embodiments 1.1 to 1.211 for use in the treatment of a cancer in a subject (e.g. a human subject) who has been screened and has been determined as suffering from a cancer which is characterised by elevated levels of PLK4 kinase (e.g. PLK4 overexpression).
3.30 A composition of matter, atropisomer or salt as defined in any one of Embodiments 1.1 to 1.211 for use in the treatment of a cancer in a subject (e.g. a human subject) who has been screened and has been determined as suffering from a cancer which is characterised by elevated levels of PLK1 kinase and PLK4 kinase (e.g. PLK1 and PLK4 overexpression).
3.31 A composition of matter, atropisomer or salt as defined in any one of Embodiments 1.1 to 1.211 for use in the treatment of a cancer in a subject (e.g. a human subject) who has been screened and has been determined as suffering from, or being at risk of suffering from, a disease or condition which would be susceptible to treatment with a compound having activity against KRAS.
3.32 A composition of matter, atropisomer or salt as defined in any one of Embodiments 1.1 to 1.211 for use in the treatment of a subject (e.g. a human subject) who has been screened and has been determined as suffering from a cancer which is one which is characterised by mutated KRAS and which would be susceptible to treatment with a compound having activity against KRAS or against cancer cells carrying a mutant KRAS.
3.33 A composition of matter, atropisomer or salt for use according to any one of Embodiments 3.28 to 3.32 wherein the cancer is a cancer as defined in any one of Embodiments 3.4 to 3.16.
3.34 The use of a composition of matter, atropisomer or salt as defined in any one of Embodiments 1.1 to 1.211 for the manufacture of a medicament for a use as defined in any one of Embodiments 3.28 to 3.33.
3.35 A method for the diagnosis and treatment of a disease state or condition (e.g. a cancer, for example a cancer as defined in any one of Embodiments 3.4 to 3.16) mediated by KRAS or characterised by the presence of a mutated form of KRAS, which method comprises (i) screening a subject (e.g. a human subject) to determine whether a disease or condition from which the subject is or may be suffering is one which would be susceptible to treatment with a compound having activity against KRAS; and (ii) where it is indicated that the disease or condition from which the subject is thus susceptible, thereafter administering to the subject a therapeutically effective amount of a composition of matter, atropisomer or salt as defined in any one of Embodiments 1.1 to 1.211.
3.36 A method for the treatment of a disease state or condition (e.g. a cancer, for example a cancer as defined in any one of Embodiments 3.4 to 3.16) characterised by the presence of a mutated form of KRAS, which method comprises administering a therapeutically effective amount of a composition of matter, atropisomer or salt as defined in any one of Embodiments 1.1 to 1.211 to a subject (e.g. a human subject) who has been screened and has been determined as suffering from, or being at risk of suffering from, a disease or condition which would be susceptible to treatment with a compound having activity against KRAS.
3.37 A method for the diagnosis and treatment of a cancer which is characterised by elevated levels of PLK1 kinase, which method comprises (i) screening a patient to determine whether a cancer from which the patient is suffering is one which is characterised by elevated levels of PLK1 kinase; and (ii) where it is indicated that the cancer is one which is characterised by elevated levels of PLK1 kinase, thereafter administering to the patient a therapeutically effective amount of a composition of matter, atropisomer or salt as defined in any one of Embodiments 1.1 to 1.211.
3.38 Use of a composition of matter, atropisomer or salt as defined in any one of Embodiments 1.1 to 1.211 for the manufacture of a medicament for the treatment or prophylaxis of a disease state or condition in a patient who has been screened and has been determined as suffering from, or being at risk of suffering from, a disease or condition which would be susceptible to treatment with a compound having activity against KRAS.

Pharmaceutical Formulations

The composition of matter or atropisomers of the invention are typically administered to patients in the form of a pharmaceutical composition. Accordingly, in another Embodiment of the invention (Embodiment 4.1), the invention provides a pharmaceutical composition comprising a composition of matter, atropisomer or salt as defined in any one of Embodiments 1.1 to 1.211 and a pharmaceutically acceptable excipient.

In further embodiments, there are provided:
4.2 A pharmaceutical composition according to Embodiment 4.1 which comprises from approximately 1% (w/w) to approximately 95% (w/w) of a composition of matter, atropisomer or salt as defined in any one of Embodiments 1.1 to 1.211 and from 99% (w/w) to 5% (w/w) of a pharmaceutically acceptable excipient or combination of excipients and optionally one or more further therapeutically active ingredients.
4.3 A pharmaceutical composition according to Embodiment 4.2 which comprises from approximately 5% (w/w) to approximately 90%,% (w/w) of a composition of matter, atropisomer or salt as defined in any one of Embodiments 1.1 to 1.211 and from 95% (w/w) to 10% of a pharmaceutically excipient or combination of excipients and optionally one or more further therapeutically active ingredients.
4.4 A pharmaceutical composition according to Embodiment 4.3 which comprises from approximately 10% (w/w) to approximately 90%,% (w/w) of a composition of matter, atropisomer or salt as defined in any one of Embodiments 1.1 to 1.211 and from 90% (w/w) to 10% of a pharmaceutically excipient or combination of excipients.
4.5 A pharmaceutical composition according to Embodiment 4.4 which comprises from approximately 20% (w/w) to approximately 90%,% (w/w) of a composition of matter, atropisomer or salt as defined in any one of Embodiments 1.1 to 1.211 and from 80% (w/w) to 10% of a pharmaceutically excipient or combination of excipients.
4.6 A pharmaceutical composition according to Embodiment 4.5 which comprises from approximately 25% (w/w) to approximately 80%,% (w/w) of a composition of matter, atropisomer or salt as defined in any one of Embodiments 1.1 to 1.211 and from 75% (w/w) to 20% of a pharmaceutically excipient or combination of excipients.

The pharmaceutical compositions of the invention can be in any form suitable for oral, parenteral, topical, intranasal, intrabronchial, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, sprays, powders, granules, elixirs and suspensions, sublingual tablets, sprays, wafers or patches and buccal patches.

Accordingly, in further embodiments, the invention provides:
4.7 A pharmaceutical composition according to any one of Embodiments 4.1 to
4.6 which is suitable for oral administration.
4.8 A pharmaceutical composition according to Embodiment 4.7 which is selected from tablets, capsules, caplets, pills, lozenges, syrups, solutions, sprays, powders, granules, elixirs and suspensions, sublingual tablets, sprays, wafers or patches and buccal patches.
4.9 A pharmaceutical composition according to Embodiment 4.8 which is selected from tablets and capsules.
4.10 A pharmaceutical composition according to any one of Embodiments 4.1 to 4.6 which is suitable for parenteral administration.
4.11 A pharmaceutical composition according to Embodiment 4.10 which is formulated for intravenous, intramuscular, intraperitoneal, subcutaneous administration or for direct delivery into a target organ or tissue by injection, infusion or other means of delivery.
4.12 A pharmaceutical composition according to Embodiment 4.11 which is a solution or suspension for injection or infusion.

Pharmaceutical compositions (e.g. as defined in any one of Embodiments 4.1 to 4.12) containing the composition of matter, atropisomer or salt as defined in any one of Embodiments 1.1 to 1.211 of the invention can be formulated in accordance with known techniques, see for example, Remington's Pharmaceutical Sciences, Mack Publishing Company, Easton, Pa., USA.

Thus, tablet compositions (as in Embodiment 4.9) can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, talc, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations (as in Embodiment 4.9) may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract Thus, the coating can be selected so as to degrade under certain pH conditions within the gastrointestinal tract, thereby selectively release the composition of matter or atropisomer in the stomach or in the ileum or duodenum.

Instead of, or in addition to, a coating, the drug can be presented in a solid matrix comprising a release controlling agent, for example a release delaying agent which may be adapted to selectively release the composition of matter or atropisomer under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral administration (as in Embodiments 4.10 to 4.12) are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped mouldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The composition of matter or atropisomers of the inventions will generally be presented in unit dosage form and, as such, will typically contain sufficient compound to provide a desired level of biological activity. For example, a according to any one of Embodiments 3.1 to 3.9), a composition intended for oral administration may contain from 2 milligrams to 200 milligrams of active ingredient, more usually from 10 milligrams to 100 milligrams, for example, 12.5 milligrams, 25 milligrams and 50 milligrams.

Posology

The active compound (a composition of matter, atropisomer or salt as defined in any one of Embodiments 1.1 to 1.211) will be administered to a patient in need thereof (for example a human or animal patient) in an amount sufficient to achieve the desired therapeutic effect: e.g. an effect as set out in Embodiments 3.1 to 3.38 above.

The composition of matter, atropisomer or salt will generally be administered to a subject in need of such administration, for example a human or animal patient, preferably a human.

The composition of mater, atropisomer or salt will typically be administered in amounts that are therapeutically or prophylactically useful and which generally are non-toxic. However, in certain situations, the benefits of administering the composition of matter, atropisomer or salt may outweigh the disadvantages of any toxic effects or side effects, in which case it may be considered desirable to administer compounds in amounts that are associated with a degree of toxicity.

A typical daily dose of the composition of matter, atropisomer or salt can be in the range from 0.025 milligrams to 5 milligrams per kilogram of body weight, for example up to 3 milligrams per kilogram of bodyweight, and more typically 0.15 milligrams to 5 milligrams per kilogram of bodyweight although higher or lower doses may be administered where required.

By way of example, an initial starting dose of 12.5 mg may be administered 2 to 3 times a day. The dosage can be increased by 12.5 mg a day every 3 to 5 days until the maximal tolerated and effective dose is reached for the individual as determined by the physician. Ultimately, the quantity of compound administered will be commensurate with the nature of the disease or physiological condition being treated and the therapeutic benefits and the presence or absence of side effects produced by a given dosage regimen, and will be at the discretion of the physician.

Combination Therapy

It is envisaged that the composition of matter, atropisomer or salt as defined in any one of Embodiments 1.1 to 1.211 will be useful either as sole chemotherapeutic agent or, more usually, in combination therapy with chemotherapeutic agents or radiation therapy in the prophylaxis or treatment of a range of proliferative disease states or conditions. Examples of such disease states and conditions are set out above.

Particular examples of chemotherapeutic agents or other treatments that may be co-administered with the composition of matter, atropisomer or salt as defined in any one of Embodiments 1.1 to 1.211:
Topoisomerase I inhibitors
Antimetabolites: (e.g. Cytarabine)
Tubulin targeting agents
DNA binder and topoisomerase II inhibitors
EGFR inhibitors (e.g. Gefitinib—see Biochemical Pharmacology 78 2009 460-468)
mTOR inhibitors (e.g. Everolimus)
PI3K pathway inhibitors (e.g. PI3K, PDK1)
Akt inhibitors
Alkylating Agents (e.g. temozolomide)
Monoclonal Antibodies.
Anti-Hormones
Signal Transduction inhibitors
Proteasome Inhibitors
DNA methyl transferase inhibitors
Cytokines and retinoids
Hypoxia triggered DNA damaging agents (e.g. Tirapazamine)
Aromatase inhibitors
Anti Her2 antibodies, (see for example http://www.wipo.int/pctdb/en/wo.jsp?wo=2007056118)
Anti cd20 antibodies
Inhibitors of angiogenesis
HDAC inhibitors
MEK inhibitors
B-Raf inhibitors
ERK inhibitors
HER2 small molecule inhibitors e.g. lapatinib
Bcr-Abl tyrosine-kinase inhibitors e.g. imatinib
CDK4/6 inhibitor e.g. Ibrance
Mps1/TTK inhibitors
Aurora B inhibitors
FLT3 kinase inhibitors
IDH1 or IDH2 inhibitors
BRD4 inhibitors
temozolomide
Inhibitors of immune checkpoint blockade signalling components including PD1, PDL-1 and CTLA4; and
radiotherapy.

Accordingly, in further embodiments, the invention provides:
5.1 A pharmaceutical combination comprising a composition of matter, atropisomer or salt as defined in any one of Embodiments 1.1 to 1.211 and another therapeutically active agent.
5.2 A pharmaceutical combination according to Embodiment 5.1 wherein the said another therapeutic agent is selected from the chemotherapeutic agents listed above.
5.3 A pharmaceutical combination according to Embodiment 5.1 wherein the said another therapeutic agent is an anticancer agent.
5.4 A pharmaceutical combination according to any one of Embodiments 5.1 to 5.3 wherein the composition of matter, atropisomer or salt as defined in any one of Embodiments 1.1 to 1.211 and the said another therapeutically active agent are presented in a single pharmaceutical composition or patient pack.
5.5 A pharmaceutical composition comprising a composition of matter, atropisomer or salt as defined in any one of Embodiments 1.1 to 1.211, another therapeutically active agent and at least one pharmaceutically acceptable excipient.
5.6 A method of treatment of a subject suffering from a cancer which method comprises the administration to the subject of a therapeutically effective amount of a pharmaceutical combination according to any one of Embodiments 5.1 to 5.5.
5.7 A composition of matter, atropisomer or salt as defined in any one of Embodiments 1.1 to 1.211 for use in enhancing a therapeutic effect of radiation therapy or chemotherapy in the treatment of a proliferative disease such as cancer.
5.8 The use of a composition of mater, atropisomer or salt as defined in any one of Embodiments 1.1 to 1.211 or a pharmaceutically acceptable salt thereof for the manufacture of a medicament for enhancing a therapeutic effect of radiation therapy or chemotherapy in the treatment of a proliferative disease such as cancer.
5.9 A method for the prophylaxis or treatment of a proliferative disease such as cancer, which method comprises administering to a patient in combination with radiotherapy or chemotherapy a composition of matter, atropisomer or salt as defined in any one of Embodiments 1.1 to 1.211 or a pharmaceutically acceptable salt thereof.

### BRIEF DESCRIPTION OF THE DRAWINGS

FIG. 1 is a schematic diagram illustrating the R/S classification system for atropisomers.

FIG. 2 is a depiction of the three dimensional structure of 2,4-[5-(4-chlorophenyl)-1-[2-(trifluoromethyl)-phenyl]pyrrol-2-yl]-N-[2-(dimethylamino)-ethyl]benzamide atropisomer A-2 as determined by single crystal X-ray crystallographic studies.

FIG. 3 is a schematic stereochemical illustration of the two atropisomers A-1 (S) and A-2 (R) and the basis for assigning their stereochemical structures using the Cahn-Ingold-Prelog (CIP) sequence rules.

FIG. 4 is an X-ray powder diffraction spectrum for atropisomer A-2 free base.

FIG. 5 is an X-ray powder diffraction spectrum for atropisomer A-2 Tartrate Pattern A salt (bottom line) and Pattern B salt (top and middle lines)

FIG. 6 illustrates the thermal profile for atropisomer A-2 free base and shows a differential scanning calorimetry plot (line 6A) and a thermo-gravimetric analysis plot (line 6B).

FIG. 7 illustrates the thermal profile for atropisomer A-2 Tartrate Pattern A salt and shows a differential scanning calorimetry plot (line 7A) and a thermo-gravimetric analysis plot (line 7B).

FIG. 8 illustrates the thermal profile for atropisomer A-2 Tartrate Pattern B salt and shows a differential scanning calorimetry plot (line 8A) and a thermo-gravimetric analysis plot (line 8B).

FIG. 9 is a plot of weight change versus relative humidity in Gravimetric Vapour Sorption studies carried out on atropisomer A-2 Tartrate Pattern B salt.

FIG. 10 is a bar chart showing the proportions of different observed mitotic phenotypes (non-congressed chromosomes, multipolar spindles/abnormal cytokinesis, monopolar spindles, normal prometaphase, normal metaphase produced after) after treating U87MG cells with 0.03 μM concentrations of either of atropisomer A-1 or atropisomer A-2.

FIG. 11 is a bar chart showing the numbers of centrioles present in HeLa cells after treatment with 0.02 μM concentrations of either of atropisomer A-1 or atropisomer A-2.

FIG. 12 is a plot of blood plasma concentrations against time following oral and i.v. dosing to mice of atropisomer A-2. The lower line, extending as far as 24 hours, is the line for the 2 mg/kg i.v. dose. The other line is for the 10 mg/kg p.o. dose.

FIG. 13 is a plot of blood plasma concentrations against time following oral and i.v. dosing to mice of atropisomer A-3. The lower line, extending as far as 24 hours, is the line for the i.v. dose. The other line is for the p.o. dose.

FIG. 14 is a plot of blood plasma and brain concentrations against time following oral dosing (10 mg/kg) to mice of atropisomer A-2. The upper line shows the brain concentrations while the lower line shows the plasma concentrations.

FIG. 15 is a plot of blood plasma and brain concentrations against time following oral dosing to mice of atropisomer A-3. The upper line shows the brain concentrations while the lower line shows the plasma concentrations.

FIG. 16 is a plot of tumour volume versus time in male athymic nude mice in a U87MG subcutaneous xenograft model after administration of atropisomer A-2.

FIG. 17 is a graphic comparison of bioluminescent signal linked to tumour growth in male athymic nude mice in a U87-Luc orthotopic xenograft model after administration of atropisomer A-2.

FIG. 18 is a plot of tumour volume versus time in male athymic nude mice in an HCT116 subcutaneous xenograft model after administration of atropisomer A-2.

FIG. 19 shows XRPD plots for atropisomer A-2 hydrochloride salt patterns A and B.

FIG. 20 shows XRPD plots for atropisomer A-2 mesylate salt.

FIG. 21 shows XRPD plots for atropisomer A-2 maleate salt patterns A and B.

FIG. 22 shows XRPD plots for atropisomer A-2 malate salt patterns A and B.

FIG. 23 shows XRPD plots for atropisomer A-2 tosylate salt pattern A.

FIG. 24 shows XRPD plots for atropisomer A-2 phosphate salt patterns A and B.

FIG. 25 shows XRPD plots for atropisomer A-2 sulfate salt patterns A and B.

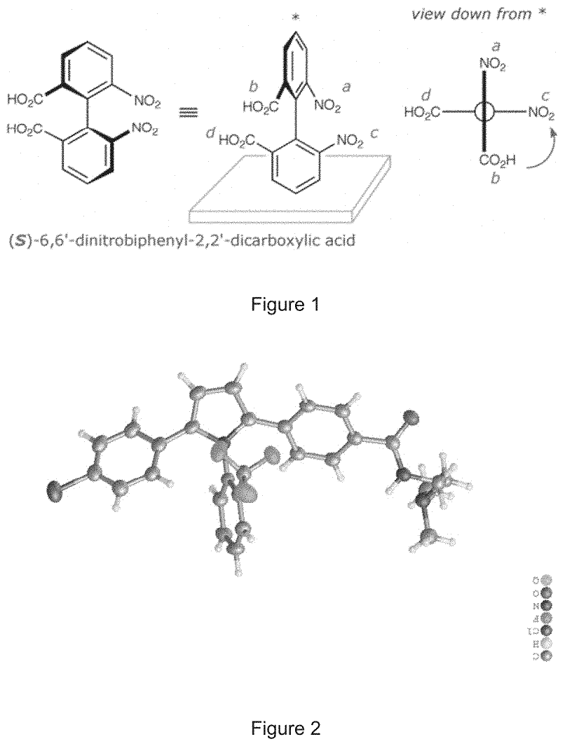

### EXAMPLES

The invention will now be illustrated, but not limited, by reference to the specific embodiments described in the following examples.

In the examples, the following abbreviations are used.
aq aqueous
CaCl₂ calcium chloride
DCM dichloromethane
DEA diethylamine
DIPEA N,N-diisopropylethylamine
DMF dimethylformamide
DMP Dess-Martin periodinane
DMSO dimethylsulfoxide
Et₂O diethyl ether
EtOAc ethyl acetate
EtOH ethanol
h hour(s)
HATU (1-[bis(dimethylamino)methylene]-1H-1,2,3-triazolo[4,5-b]pyridinium-3-oxid hexafluorophosphate)
HCl hydrogen chloride
HPLC high performance liquid chromatography
H₂SO₄ sulfuric acid
IPA iso-propanol
LC liquid chromatography
LCMS liquid chromatography-mass spectrometry
UOH lithium hydroxide
MeCN acetonitrile
MeOH methanol
min minute(s)
MTBE methyl tert-butyl ether
NaBH₄ sodium borohydride
NaHCO₃ sodium hydrogen carbonate
NaOH sodium hydroxide
Na₂SO₄ sodium sulfate
NH₄Cl ammonium chloride
NMR nuclear magnetic resonance
PTSA p-toluenesulfonic acid
TEA triethylamine
THF tetrahydrofuran

### COMPOUND DETAILS AND EXPERIMENTAL

Atropisomers A-1 to A-8

| | | Chemical name | |
|---|---|---|---|
| Atropisomer | Structure | (IUPAC via ISIS draw) | Notes |
| A-1 | 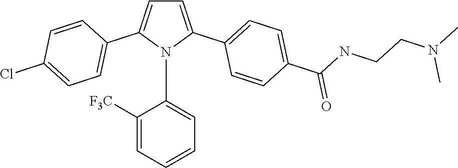 [SMILES: CN(C)CCNC(=O)c1ccc(-c2ccc(-c3ccc(Cl)cc3)n2-c2ccccc2C(F)(F)F)cc1] | 4-[5-(4-chlorophenyl)-1- [2-(trifluoromethyl)- phenyl]pyrrol-2-yl]-N-[2- (dimethylamino)- ethyl]benzamide- atropisomer 1 | S-atropisomer |
| A-2 | 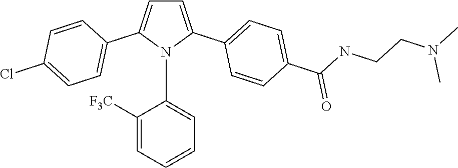 [SMILES: CN(C)CCNC(=O)c1ccc(-c2ccc(-c3ccc(Cl)cc3)n2-c2ccccc2C(F)(F)F)cc1] | 4-[5-(4-chlorophenyl)-1- [2-(trifluoromethyl)- phenyl]pyrrol-2-yl]-N-[2- (dimethylamino)- ethyl]benzamide- atropisomer 2 | R-atropisomer |
| A-3 | 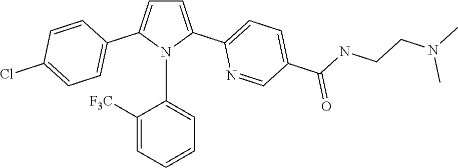 [SMILES: CN(C)CCNC(=O)c1ccc(-c2ccc(-c3ccc(Cl)cc3)n2-c2ccccc2C(F)(F)F)nc1] | 6-[5-(4-chlorophenyl)-1- [2-(trifluoromethyl)- phenyl]pyrrol-2-yl]-N-[2- (dimethylamino)- ethyl]pyridine-3- carboxamide- atropisomer 1 | Single atropisomer, unknown configuration |
| A-4 | 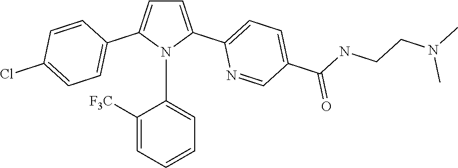 [SMILES: CN(C)CCNC(=O)c1ccc(-c2ccc(-c3ccc(Cl)cc3)n2-c2ccccc2C(F)(F)F)nc1] | 6-[5-(4-chlorophenyl)-1- [2-(trifluoromethyl)- phenyl]pyrrol-2-yl]-N-[2- (dimethylamino)- ethyl]pyridine-3- carboxamide- atropisomer 2 | Single atropisomer, unknown configuration |
| A-5 | 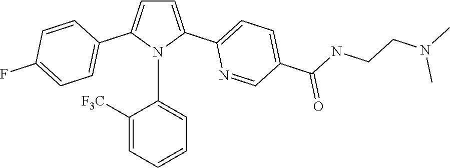 [SMILES: CN(C)CCNC(=O)c1ccc(-c2ccc(-c3ccc(F)cc3)n2-c2ccccc2C(F)(F)F)nc1] | N-[2-(dimethylamino)- ethyl]-6-[5-(4- fluorophenyl)-1-[2- (trifluoromethyl)- phenyl]pyrrol-2- yl]pyridine-3- carboxamide | Racemic mixture of both atropisomers |
| A-6 | 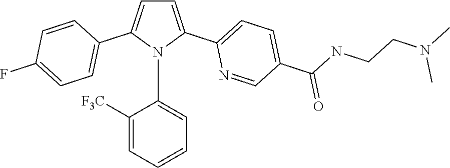 [SMILES: CN(C)CCNC(=O)c1ccc(-c2ccc(-c3ccc(F)cc3)n2-c2ccccc2C(F)(F)F)nc1] | N-[2-(dimethylamino)- ethyl]-6-[5-(4- fluorophenyl)-1-[2- (trifluoromethyl)- phenyl]pyrrol-2- yl]pyridine-3- carboxamide | Racemic mixture of both atropisomers |
| A-7 | 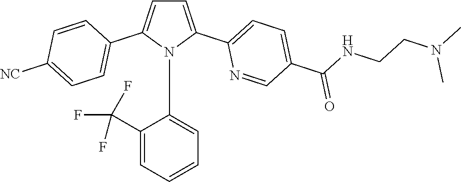 [SMILES: CN(C)CCNC(=O)c1ccc(-c2ccc(-c3ccc(C#N)cc3)n2-c2ccccc2C(F)(F)F)nc1] | 6-[5-(4-cyanophenyl)-1- [2-(trifluoromethyl)- phenyl]pyrrol-2-yl]-N-[2- (dimethylamino)- ethyl]pyridine-3- carboxamide | Racemic mixture of both atropisomers |
| A-8 | 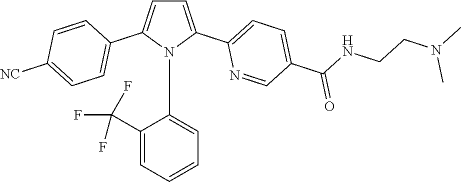 [SMILES: CN(C)CCNC(=O)c1ccc(-c2ccc(-c3ccc(C#N)cc3)n2-c2ccccc2C(F)(F)F)nc1] | 6-[5-(4-cyanophenyl)-1- [2-(trifluoromethyl)- phenyl]pyrrol-2-yl]-N-[2- (dimethylamino)- ethyl]pyridine-3- carboxamide | Racemic mixture of both atropisomers |

Proton magnetic resonance (1H NMR) spectra were recorded on a Bruker 400 instrument operating at 400 MHz, in DMSO-d6 or MeOH-d4 (as indicated) at 27° C., unless otherwise stated and are reported as follows: chemical shift 6/ppm (multiplicity where s=singlet, d=doublet, dd=double doublet, dt—double triplet, t=triplet, q=quartet, m=multiplet, br=broad, number of protons). The residual protic solvent was used as the internal reference.

Liquid chromatography and mass spectroscopy analyses were carried out using the system and operating conditions set out below. Where atoms with different isotopes are present and a single mass quoted, the mass quoted for the compound is the monoisotopic mass (i.e. ³⁵Cl; ⁷⁹Br etc.)

LCMS Conditions

The LCMS data given in the following examples were obtained using one of the methods described below.

LCMS Method 1

LCMS was carried out on UPLC AQUITY with PDA photodiode array detector and QDa mass detector. The column used was a C18, 2.1×50 mm, 1.9 μm. The column flow was 1.2 mL/min and the mobile phase used was: (A) 0.1% Formic acid in MilliQ water (pH=2.70) (B) 0.1% Formic acid in water:MeCN (10:90), the injection volume was between 4 and 7 μL. The sample was prepared in MeOH:MeCN to achieve an approximate concentration of 250 ppm.

The following gradient was used for the elution:

| Time (min) | Flow (ml/min) | % A | % B |
|---|---|---|---|
| 0.00 | 0.8 | 97 | 3 |
| 0.20 | 0.8 | 97 | 3 |
| 2.70 | 0.8 | 2 | 98 |
| 3.00 | 1.0 | 00 | 100 |
| 3.50 | 1.0 | 00 | 100 |
| 3.51 | 0.8 | 97 | 3 |
| 4.00 | 0.8 | 97 | 3 |

Mass Parameters

Probe: ESI capillary

### Source Temperature: 120° C.

### Probe Temperature: 600° C.

### Capillary Voltage: 0.8 KV (+Ve and −Ve)

### Cone Voltage: 10 & 30 V

Mode of Ionization: Positive and negative

LCMS Method 2

LCMS was carried out on Agilent Infinity II G6125C LCMS. The column used was an XBridge C18, 50×4.6 mm, 3.5 μm. The column flow was 1.0 mL/min and the mobile phase used was: (A) 5 mM Ammonium Bicarbonate in Milli-Qwater and (B) MeOH. The injection volume was 5 μL. The sample was prepared in water MeCN to achieve an approximate concentration of 250 ppm.

The following gradient was used for the elution.

| Time (min) | % A | % B |
|---|---|---|
| 0.00 | 92 | 8 |
| 0.75 | 92 | 8 |
| 3.00 | 30 | 70 |
| 3.70 | 5 | 95 |
| 4.20 | 0 | 100 |
| 5.20 | 0 | 100 |
| 5.21 | 92 | 8 |
| 7.00 | 92 | 8 |

Mass Parameter

### Ion Source: MMI

Fragmentation voltage: 70V
Mode of Ionization: Positive and negative

### Gas Temperature: 250° C.

### Vaporizer 160° C.

Gas flow: 10 L/min

### Nebulizer Pressure: 45 psi

HPLC Method 1

HPLC analysis was carried out on an Agilent Technologies 1100/1200 series HPLC system. The column used was an ACE 3 C18; 150×4.6 mm, 3.0 μm particle size (Ex: Hichrom, Part number ACE-111-1546). The flow rate was 1.0 mL/min. Mobile phase A was Water:Trifluoroacetic acid (100:0.1%) and mobile phase B was Acetonitrile:Trifluoroacetic acid (100:0.1%). The injection volume was 5 μL and the following gradient was used:

| Time (mins) | % A | % B |
|---|---|---|
| 0 | 80 | 20 |
| 35 | 5 | 95 |
| 39.5 | 5 | 95 |
| 40 | 80 | 20 |

Chiral HPLC Analysis

The chiral HPLC data reported were obtained using one of the methods described below.

Chiral HPLC Method 1

Chiral HPLC was analysis was carried out on an Agilent Technologies 1200 series HPLC system. The column used was a CHIRAL PAK IG, 250×4.6 mm, 5 μm. The column flow rate was 1.0 mL/min and the mobile phase was: (A) 0.1% v/v DEA in n-heptane and (B) IPA:MeOH (70:30). The injection volume was 25 μL. Samples were prepared in IPA:MeOH to achieve an approximate concentration of 250 ppm and with the following isocratic method:

| Time | Flow | % A | % B |
|---|---|---|---|
| 0.01 | 1.0 mL/min | 90 | 10 |
| 45 | 1.0 mL/min | 90 | 10 |

Chiral HPLC Method 2 Chiral HPLC was analysis was carried out on an Agilent Technologies 1200 series HPLC system. The column used was a CHIRALPAK IG SFC, 21×250 mm, 5 μm. The column flow rate was 1.0 mL/min and the mobile phase was: (A) 0.1% v/v DEA in n-heptane and (B) IPA:MeOH (70:30). The injection volume was 20 μL. Samples were prepared in IPA:MeOH to achieve an approximate concentration of 250 ppm and with the following isocratic method:

| Time | Flow | % A | % B |
|---|---|---|---|
| 0.01 | 1.0 mL/min | 85 | 15 |
| 30 | 1.0 mL/min | 85 | 15 |

Chiral HPLC Method 3

Chiral HPLC was carried out on an Agilent Technologies 1200 series HPLC system. The column used was a CHIRAL PAK IG, 250×4.6 mm, 5 μm. The column flow rate was 1.0 mL/min and the mobile phase was: (A) 0.1% v/v DEA in n-heptane and (B) IPA:MEOH (70:30). The injection volume was 10 μL Samples were prepared in IPA:MeCN to achieve an approximate concentration of 250 ppm and with the following isocratic method:

| Time | Flow | % A | % B |
|---|---|---|---|
| 0.01 | 1.0 mL/min | 85 | 15 |
| 25 | 1.0 mL/min | 85 | 15 |

Chiral HPLC Method 4

Identical conditions to chiral method 3 except using the following isocratic method:

| Time | Flow | % A | % B |
|---|---|---|---|
| 0.01 | 1.0 mL/min | 70 | 30 |
| 25 | 1.0 mL/min | 70 | 30 |

Chiral HPLC Method 5

Identical conditions to chiral method 3 except using the following isocratic method:

| Time | Flow | % A | % B |
|---|---|---|---|
| 0.01 | 1.0 mL/min | 90 | 10 |
| 25 | 1.0 mL/min | 90 | 10 |

Chiral HPLC Method 7

Chiral HPLC was analysis was carried out on an Agilent Technologies 1100/1200 series HPLC system. The column used was a CHIRALPAK IA; 250×4.6 mm, 5.0 μm. The column flow rate was 1.0 mL/min and the mobile phase was: Hexane:EtOH:Ethanolamine (90:10:0.1%). The injection volume was 5 μL. Samples were prepared in 100% EtOH to achieve an approximate concentration of 0.5 mg/mL.

Preparative HPLC Methods

Final compounds were purified using one of the following preparative HPLC methods.

Preparative HPLC Method 1

Preparative HPLC was carried out using a SUNFIRE Prep C18 OBD, 19×250 mm, 5 μm column with (A) 0.05% HCl in water and (B) 100% MeCN as mobile phase and a flow rate of 17 mL/min and with the following isocratic system for the elution:

| Time (min) | Flow | % A | % B |
|---|---|---|---|
| 00.01 | 17 | 70 | 30 |
| 16.00 | 17 | 57 | 43 |
| 16.01 | 17 | 2 | 98 |
| 18.00 | 17 | 2 | 98 |
| 18.01 | 17 | 70 | 30 |
| 20.00 | 17 | 70 | 30 |

Preparative HPLC Method 2

Preparative HPLC was carried out using an X-bridge prep, C18, 30×250 mm, 5 μm column with (A) 0.05% HCl in water and (B) 100% MeCN as mobile phase and a flow rate of 25 mL/min with the following isocratic system for the elution:

| Time (min) | Flow | % A | % B |
|---|---|---|---|
| 00.01 | 25 | 80 | 20 |
| 15.00 | 25 | 20 | 80 |
| 15.01 | 25 | 2 | 98 |
| 17.00 | 25 | 2 | 98 |
| 17.01 | 25 | 80 | 20 |
| 19.00 | 25 | 80 | 20 |

Preparative Chiral HPLC Methods:

The atropisomers were isolated using one of the following preparative chiral HPLC methods.

Preparative Chiral HPLC Method 1

Preparative chiral HPLC was carried out using a CHIRALPAK IG SFC, 21×250 mm, 5 μm column, eluting with (A) 0.1% DEA in heptane and (B) IPA as mobile phase, with the flow rate of 30 mL/min and the following isocratic system:

| Time (min) | % A | % B |
|---|---|---|
| 0.01 | 94 | 6 |
| 50.00 | 94 | 6 |

Preparative Chiral HPLC Method 2

Preparative chiral HPLC was carried out using a CHIRALPAK IG SFC column, 21×250 mm, 5 μm eluting with (A) 0.1% DEA in heptane and (B) IPA:MeOH (90:10) as mobile phase and a flow rate of 22 mL/min and with the following isocratic system was used for the elution:

| Time (min) | % A | % B |
|---|---|---|
| 0.01 | 93 | 7 |
| 35.00 | 93 | 7 |

Chiral Analysis Specific Optical Rotation Protocol

### Instrumentation: Optical Activity AA-10 Automatic Polarimeter

### Wavelength: 589 nm

### Temperature: 23° C.

Pathlength of cell: 1 dm
Solvent: Chloroform (Fisher, HPLC grade)
Concentration: 1.0 g/100 mL

Sampling Technique

The instrument was switched on and allowed to stabilize for 30 minutes before calibration was checked using an Optical Activity Quartz Control Plate (S/N 00049). The angular rotation at 23° C. using sodium yellow D line was measured at 34.16° (after firstly zeroing the instrument without any sample tube). The sample tube quality was then checked by zeroing the instrument, then filling the sample tube with chloroform and checking the instrument was still reading 0.00 (+/−0.02). The instrument was zeroed with the chloroform blank in place.

The sample was dissolved in CHCl₃ (2 mg in 2 mL), filtered and 2 mL was pipetted into the cell to measure α.

The specific optical rotation was calculated from the following equation: [α]Tλ=(α×100)/(cl)

### Synthesis of Intermediates

### Intermediate A: 1-(4-chlorophenyl)-3-(dimethylamino) propan-1-one hydrochloride

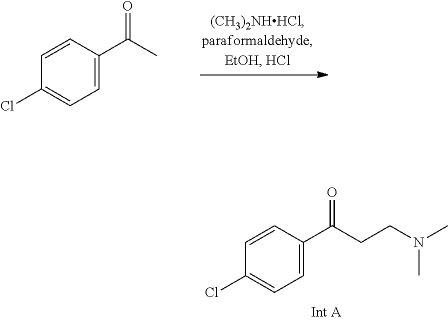

To a solution of 4′-chloroacetophenone (10 g, 65 mmol) in absolute EtOH (50 mL) at room temperature were added paraformaldehyde (1.94 g, 64 mol), N,N-dimethylamine hydrochloride (5.27 g, 64.68 mmol) and conc. HCl (2 mL). The resulting reaction mixture was stirred at between 80-90° C. for 30 h. The reaction mixture was concentrated under reduced pressure and the resulting residue was purified by column chromatography with silica gel (60-120 mesh) eluting with 2% EtOAc/hexane) and trituration with Et₂O (100 mL) to afford the title compound (10 g, 40 mmol, 62%).

### Intermediate B: 3-(dimethylamino)-1-(4-fluorophenyl) propan-1-one hydrochloride

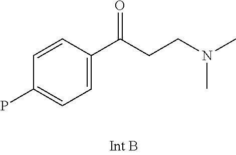

Intermediate B was prepared using the same method as described for intermediate A except that 4′-fluoroacetophenone (20 g, 144.87 mmol) was used and the resulting residue was purified by column chromatography with silica gel (60-120 mesh) eluting with 4% MeOH/DCM) followed by trituration with Et₂O (400 mL) to afford the title compound (15 g, 77 mmol, 53%).

### Intermediate C: 4-(3-(dimethyl amino) propanol) benzonitrile hydrochloride

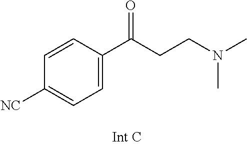

Intermediate C was prepared using the same method as described for intermediate A except that 4-acetylbenzonitrile (25 g, 172 mmol) was used and the resulting residue was purified by column chromatography with silica gel (60-120 mesh) eluting with 5% MeOH/DCM followed by trituration with Et₂O (400 mL) to afford the title compound (20 g, 99 mmol, 57%).

### Example 1

### Preparation of Atropisomers A-1 and A-2

Atropisomers A-1 and A-2 can be prepared by following Synthetic Route A as shown below.

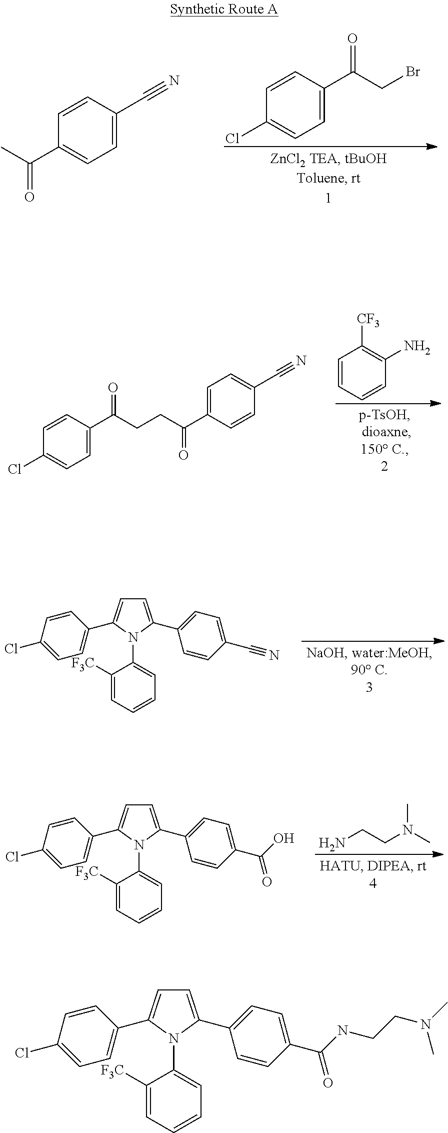

### Step 1: 4-[4-(4-chlorophenyl)-4-oxo-butanoyl]benzonitrile

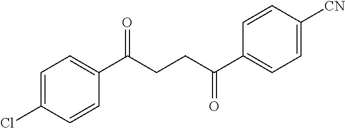 [SMILES: N#Cc1ccc(C(=O)CCC(=O)c2ccc(Cl)cc2)cc1]

Zinc chloride (30.5 g, 223 mmol) was heated to melting under vacuum then cooled to room temperature. Toluene (100 mL), tert-butanol (16.5 mL, 172 mmol) and TEA (24 mL, 172 mmol) and the mixture stirred at room temperature for 2 h under a nitrogen atmosphere at which point the zinc chloride had fully dissolved. 4-Cyanoacetophenone (25 g, 172 mmol) and 4-chlorophenacylbromide (40.2 g, 172 mmol) were added and the reaction mixture was stirred at room temperature for 48 h. The reaction mixture was diluted with EtOAc (300 mL) and washed with water (5×100 mL). The combined organic extracts were dried (Na₂SO₄) and evaporated under reduced pressure. The resulting residue was purified by trituration using MTBE (400 mL) to afford the title compound (30 g, 101 mmol, 59%).

### Step 2: 4-(5-(4-chlorophenyl)-1-(2-(trifluoromethyl) phenyl)-1H-pyrrol-2-yl) benzonitrile

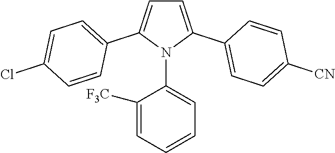 [SMILES: N#Cc1ccc(-c2ccc(-c3ccc(Cl)cc3)n2-c2ccccc2C(F)(F)F)cc1]

A stirred solution of 4-(4-(4-chlorophenyl)-4-oxobutanoyl) benzonitrile (30 g, 101 mmol), 2-trifluoromethyl aniline (48.79 g, 303 mmol) and PTSA (1.92 g, 10.099 mmol) in dioxane (300 mL) was heated at 150° C. for 16 h. The reaction mixture concentrated under reduced pressure and the resulting residue was purified by column chromatography on silica gel (60-120 mesh) using 8% EtOAc/hexane as the eluent to afford the title compound (30 g, 71 mmol, 70%).

### Step 3: 4-(5-(4-chlorophenyl)-1-(2-(trifluoromethyl) phenyl)-1H-pyrrol-2-yl) benzoic acid

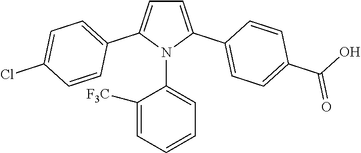 [SMILES: O=C(O)c1ccc(-c2ccc(-c3ccc(Cl)cc3)n2-c2ccccc2C(F)(F)F)cc1]

To a solution of 4-(5-(4-chlorophenyl)-1-(2-(trifluoromethyl) phenyl)-1H-pyrrol-2-yl) benzonitrile (2 g, 4.739 mmol) in MeOH (20 mL) was added NaOH (1.89 g, 47 mmol) in water (10 mL) and the resulting mixture was stirred at 90° C. for 24 h. The mixture was concentrated under reduced pressure and the resulting residue was purified by trituration by using Et₂O (10 mL) to afford the title compound (1.8 g, 4.1 mmol, 86%).

### Step 4: 4-(5-(4-chlorophenyl)-1-(2-(trifluoromethyl) phenyl)-1H-pyrrol-2-yl)-N-(2-(dimethylamino) ethyl) benzamide

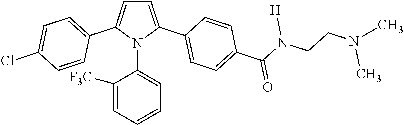 [SMILES: CN(C)CCNC(=O)c1ccc(-c2ccc(-c3ccc(Cl)cc3)n2-c2ccccc2C(F)(F)F)cc1]

To a stirred solution of 4(5-(4-chlorophenyl)-1-(2-(trifluoromethyl)phenyl)-1H-pyrrol-2-yl)benzoic acid (1.8 g, 4.0 mmol) in DMF (12 mL) was added DIPEA (2.13 mL, 22 mmol) followed by HATU (4.65 g, 12 mmol). The reaction mixture was stirred at room temperature for 30 min followed by the addition of N,N′-dimethylethylenediamine (1.08 g, 12 mmol) dropwise and stirring continued at room temperature for 4 h. The mixture was poured into ice-cold water (150 mL) and extracted with EtOAc (3×100 mL). The combined organic layers were dried (Na₂SO₄) and concentrated under reduced pressure. The resulting residue was purified by column chromatography on neutral Alumina eluting with 6% MeOH/DCM to afford the title compound (1.2 g, 2.3 mmol, 57%) as a mixture of atropisomers.

Separation of Atropisomers

The atropisomers (A-1 and A-2) of 4-[5-(4-chlorophenyl)-1-[2-(trifluoromethyl)phenyl]pyrrol-2-yl]-N-[2-(dimethylamino)ethyl]benzamide may be resolved by chiral HPLC using preparative chiral HPLC method 1.

Two peaks were isolated:
Peak 1: Atropisomer A-1, 4-[5-(4-chlorophenyl)-1-[2-(trifluoromethyl)phenyl]pyrrol-2-yl]-N-[2 (dimethylamino)ethyl]benzamide-atropisomer1 (0.3 g, 0.58 mmol, 38%, >99% ee), and:
Peak 2: Atropisomer A-2, 4-[5-(4-chlorophenyl)-1-[2-(trifluoromethyl)phenyl]pyrrol-2-yl]-N-[2 (dimethylamino)ethyl]benzamide-atropisomer2 (0.31 g, 0.606 mmol, 39%, 98% ee).

The compounds can also be isolated as their hydrochloride salts.

### Example 2

Further purification and characterisation of the atropisomers

### Atropisomer A-1: 4-[5-(4-chlorophenyl)-1-[2-(trifluoromethyl)phenyl]pyrrol-2-yl]-N-[2 (dimethylamino)ethyl]benzamide hydrochloride salt

Peak 1 (0.31 g, 0.606 mmol) was further purified by stirring in HPLC grade water (30 mL) followed by sonication for 10 min and extraction with EtOAc (3×30 mL). The combined organic layers were dried (Na₂SO₄), filtered and concentrated under reduced pressure followed by lyophilisation to afford an amorphous solid (0.290 g, 0.567 mmol, 94%) which was dissolved in DCM (7.12 mL). The resulting solution was cooled to 0° C. and 4N HCl in dioxane (1.42 mL) was added. The reaction mixture was stirred at room temperature for 3 h. The mixture was concentrated and dried under high vacuum. Purification by trituration using Et₂O (10 mL) and lyophilisation afforded the title compound (0.3 g, 0.56 mmol, 98%) as an off-white solid.

¹H NMR (DMSO-d₆) δ 10.03, (brs, 1H), 8.62 (s, 1H), 7.81-7.68 (m, 6H), 7.25 (d, J=8.4 Hz, 2H), 7.10-7.03 (m, 4H), 6.67-6.58 (m, 2H), 3.56-3.54 (m, 2H), 3.20-3.18 (m, 2H), 2.76 (s, 6H). LCMS (Method 1)—RT 2.54, MH+ 512.4

### Atropisomer A-2: 4-[5-(4-chlorophenyl)-1-[2-(trifluoromethyl)phenyl]pyrrol-2-yl]-N-[2 (dimethylamino)ethyl]benzamide hydrochloride salt

The hydrochloride salt of atropisomer A-2 was prepared using the same method as used for atropisomer A-1 starting from peak 2 to afford the title compound (0.31 g, 0.56 mmol, 99%) an off-white solid.

¹H NMR (DMSO-d₆) δ 9.91 (brs, 1H), 8.69 (s, 1H), 7.81-7.68 (m, 6H), 7.25 (d, J=8.0 Hz, 2H), 7.10-7.03 (m, 4H), 6.67-6.58 (m, 2H), 3.56-3.54 (m, 2H), 3.20-3.18 (m, 2H), 2.77 (s, 6H). LCMS (Method 1)—RT 2.56, MH+ 512.4

Single crystal X-ray crystallographic analysis of atropisomer A-2 (see Example 3 below) indicated that atropisomer A-2 is the R-isomer (Compound (1)) and hence atropisomer A-1 must be the S-isomer.

Chiral Analysis

Analysis of the chiral properties of the Atropisomers A-1 and A-2 was carried out by measuring their optical rotations and their retention times obtained by chiral HPLC using the methods described above to give the results shown in the table below.

| | Chiral HPLC RT | Chiral HPLC | Specific Optical |
|---|---|---|---|
| Atropisomer | (min) | Method | Rotation |
| A-1 (S-atropisomer) | 17.063 | 1 | +12.39° |
| A-2 (R-atropisomer) | 20.553 | 1 | −11.76° |

Atropisomer Classification

Stability studies were carried out on the isolated atropisomers, atropisomers A-1 and A-2.

To assess the interconversion of atropisomer A-1 and atropisomer A-2 chiral stability was monitored at 40° C. and 80° C. As shown by the results set out below, no interconversion was observed on heating for 10 days at either temperature.

| | % ee of sample @ 40° C. | | % ee sample @ 80° C. | |
|---|---|---|---|---|
| Time/h | A-1 | A-2 | A-1 | A-2 |
| 0 | 100 | 97.20 | 100 | 97.20 |
| 24 | 100 | 97.34 | 100 | 95.62 |
| 48 | 100 | 97.30 | nd | nd |
| 72 | 100 | 97.24 | nd | nd |
| 96 | 100 | 97.14 | 100 | 96.62 |
| 10 days | 100 | 97.46 | 100 | 97.12 |

Protocol:

1. 2×1 mg of pure atropisomer was dissolved in 1 mL of EtOH in a sealed-dram vial.

2. One set of vials was heated at 40° C. and another set at 80° C.

3. At specified time-points a 20 μL aliquot from each stock solution (1 mL) was taken and quenched into a HPLC vial containing a 80 μL solution of hexane:EtOH; 80:20 to afford a final concentration of 200 ppm and the sample was analysed by chiral HPLC

4. Analysis was carried out at the following timepoints: 0 h, 24 h, 48 h, 72 h, 96 h and 240 h for the samples kept at 40° C. and 24 h, 96 h and 240 h for the samples kept at 80° C. using Chiral HPLC method 5

The stabilities of the isolated atropisomers, Example A-1 and A-2, confirmed that they are Class 3 atropisomers (LaPlante et al., J. Med. Chem., 54:7005-7022 (2011))).

### Example 3

X-Ray Crystallographic Analysis of Atropisomer A-2

Atropisomer A-2 free base was prepared, and a single crystal was subjected to X-ray crystallographic studies as described below.

### EXPERIMENTAL

Single non-defined morphology crystals of atropisomer A-2 were obtained by recrystallisation from methyl isobutyl ketone (MIBK). A suitable crystal 0.19×0.13×0.04 mm³ was selected and, using MiTiGen MicroMount, mounted on a Rigaku XtaLAB Syngery-S diffractometer equipped with a HyPix-6000HE detector. The crystal was kept at a steady T=123(2) K during data collection.

Data were generated using CuKα radiation. The maximum resolution that was achieved was Θ=74.263° (0.80 Å). Data reduction, scaling and absorption corrections were performed. The final completeness was 100.00% out to 74.263° in Θ. The absorption coefficient μ of the compound was determined as being 1.761 mm⁻¹ at the wavelength (λ=1.542 Å).

The data were collected and processed using CrysAlisPro software and the structure was solved with the SheIXT (Sheldrick, 2015) structure solution program using the Intrinsic Phasing solution method and by using Olex2 (Dolomanov et al., 2009) as the graphical interface. The model was refined with version 2018/3 of SheIXL-2018/3 (Sheldrick, 2018) using Least Squares minimisation.

The crystal structure was found to be monoclinic and was assigned the space group P21 (#4).

All non-hydrogen atoms were refined anisotropically. Hydrogen atom positions were calculated geometrically and refined using the riding model.
References: O. V. Dolomanov and L. J. Bourhis and R. J. Gildea and J. A. K. Howard and H. Puschmann, Olex2: A complete structure solution, refinement and analysis program, J. Appl. Cryst., (2009), 42, 339-341.
Sheldrick, G. M., Crystal structure refinement with SheIXL, Acta Cryst., (2015), C71, 3-8.
Sheldrick, G. M., SheIXT-Integrated space-group and crystal-structure determination, Acta Cryst., (2015), A71, 3-8.

The results of the studies are set out below in Tables 1-7.

TABLE 1
Data for crystal of Atropisomer A-2 Free Base

| Formula | C₂₈H₂₅CIF₃N₃O |
|---|---|
| D_{calc.}/g cm⁻³ | 1.350 |
| m/mm⁻¹ | 1.761 |
| Formula Weight | 511.96 |
| Colour | n/a |
| Shape | n/a |
| Size/mm³ | 0.19 × 0.13 × 0.04 |
| T/K | 123(2) |
| Crystal System | monoclinic |
| Flack Parameter | −0.03(2) |
| Hooft Parameter | −0.020(6) |
| Space Group | P2₁ |
| a/Å | 10.1964(3) |
| b/Å | 8.6349(4) |
| c/Å | 14.3398(6) |
| a/° | 90 |
| b/° | 93.955(4) |
| g/° | 90 |
| v/Å³ | 1259.53(9) |
| Z | 2 |
| Z′ | 1 |
| Wavelength/Å | 1.54184 |
| Radiation type | CuKₐ |
| Qₘᵢₙ/° | 3.089 |
| Qₘₐₓ/° | 74.263 |
| Measured Refl. | 17242 |
| Independent Refl. | 4929 |
| Reflections with I > 2(I) | 4544 |
| Rᵢₙₜ | 0.0357 (3.57 %) |
| Parameters | 327 |
| Restraints | 1 |
| Largest Peak | 0.381 |
| Deepest Hole | −0.188 |
| GooF | 1.032 |
| wR₂ (all data) | 0.1228 |
| wR₂ | 0.1174 |
| R₁ (all data) | 0.0474 |
| R₁ | 0.0433 |
| Reflections | d min (Cu) = 0.80; I/σ = 35.2; |
| | Complete 10% (IUCR) = 99% |
| Refinement | Shift = 0.000; |
| | Max. Peak = 0.4; Min peak = −0.2 |

TABLE 2
Fractional Atomic Coordinates (×104) and Equivalent
Isotropic Displacement Parameters (Å² × ¹⁰³) for
Atropisomer A-2. U_{eq} is defined as ⅓ of
the trace of the orthogonalised Uᵢⱼ.

| Atom | x | y | z | U_{eq} |
|---|---|---|---|---|
| CI36 | 7764.8(9) | 10484.3(12) | 2175.1(6) | 58.8(3) |
| F19 | 8576.0(19) | 5604(3) | 7787.5(13) | 47.5(5) |
| F20 | 9300.0(18) | 6815(3) | 6631.6(14) | 56.0(6) |
| F21 | 8706(2) | 8065(3) | 7827.5(18) | 61.5(6) |
| 035 | 6134(2) | -381(3) | 9646.9(18) | 44.1(5) |
| N29 | 4745(2) | 1641(3) | 9788.3(18) | 36.4(5) |
| N32 | 1997(2) | 656(3) | 9424.4(17) | 36.5(5) |
| N7 | 7412(2) | 4971(3) | 5673.0(16) | 32.3(5) |
| C28 | 5700(3) | 889(3) | 9377(2) | 34.9(6) |
| C12 | 6589(3) | 6128(3) | 6040(2) | 31.4(6) |
| C11 | 7755(3) | 3554(4) | 6084(2) | 35.0(6) |
| C9 | 8822(3) | 3840(4) | 4773(2) | 39.7(7) |
| C17 | 7018(3) | 7016(4) | 6817(2) | 34.2(6) |
| C13 | 5360(3) | 6390(4) | 5597(2) | 36.7(6) |
| C26 | 7530(3) | 1442(4) | 8352(2) | 36.1(6) |
| C2 | 7880(3) | 8040(4) | 4542(2) | 37.9(6) |
| C8 | 8077(3) | 5149(4) | 4861(2) | 35.3(6) |
| C1 | 7975(3) | 6504(4) | 4243(2) | 36.4(6) |
| C24 | 5413(3) | 2530(4) | 7922(2) | 35.7(6) |
| C18 | 8397(3) | 6877(4) | 7264(2) | 37.7(6) |
| C22 | 7203(3) | 2948(4) | 6928(2) | 34.9(6) |
| C3 | 7834(3) | 9267(4) | 3916(2) | 41.2(7) |
| C25 | 6226(3) | 1670(3) | 8545(2) | 33.5(6) |
| C31 | 2911(3) | -109(4) | 10106(2) | 38.8(7) |
| C6 | 8027(3) | 6246(4) | 3276(2) | 40.6(7) |
| C23 | 5889(3) | 3149(4) | 7118(2) | 36.8(6) |
| C30 | 4005(3) | 939(4) | 10513(2) | 38.6(7) |
| C27 | 8013(3) | 2085(4) | 7566(2) | 36.1(6) |
| C10 | 8632(3) | 2863(4) | 5533(2) | 39.2(7) |
| C16 | 6185(3) | 8123(4) | 7156(2) | 42.2(7) |
| C15 | 4949(4) | 8367(4) | 6710(3) | 46.4(8) |
| C4 | 7882(3) | 8953(5) | 2972(2) | 42.5(7) |
| C14 | 4541(3) | 7517(4) | 5926(3) | 41.6(7) |
| C5 | 7986(3) | 7463(5) | 2643(2) | 43.4(8) |
| C34 | 1206(3) | 1831(5) | 9855(2) | 47.0(8) |
| C33 | 1136(4) | -484(5) | 8950(3) | 49.5(8) |

TABLE 3
Anisotropic Displacement Parameters (×10⁴) SOL_686_i42-5 Hz.
The anisotropic displacement factor exponent takes the form:
−2π²[h²a*² × U₁₁ + . . . + 2hka* × b* × U₁₂]

| Atom | U₁₁ | U₂₂ | U₃₃ | U₂₃ | U₁₃ | U₁₂ |
|---|---|---|---|---|---|---|
| Cl36 | 57.6(5) | 68.4(6) | 49.6(4) | 19.0(4) | −1.6(4) | −1.6(4) |
| F19 | 46.7(10) | 46.1(11) | 48.0(10) | 7.6(9) | −10.0(8) | −6.8(9) |
| F20 | 31.1(9) | 89.9(17) | 46.4(10) | 9.7(11) | −1.4(7) | −10.4(10) |
| F21 | 62.3(13) | 47.5(12) | 70.7(14) | −13.8(11) | −24.9(11) | −5.3(11) |
| O35 | 43.0(12) | 35.0(11) | 54.6(13) | 7.3(10) | 4.8(10) | 3.5(10) |
| N29 | 33.7(12) | 32.3(12) | 43.3(13) | 2.5(11) | 2.4(10) | −1.1(10) |
| N32 | 33.3(12) | 36.4(13) | 39.4(12) | −0.1(11) | −0.2(10) | −1.4(11) |
| N7 | 25.9(11) | 36.3(13) | 34.6(11) | −1.4(10) | 1.9(8) | 0.6(9) |
| C28 | 31.2(13) | 29.4(14) | 43.5(14) | 0.1(12) | −1.6(11) | −2.7(11) |
| C12 | 28.5(12) | 31.1(14) | 35.0(13) | 0.7(11) | 4.2(10) | −1.3(10) |
| C11 | 25.8(13) | 38.8(16) | 39.9(15) | −2.0(12) | −1.8(11) | 0.4(11) |
| C9 | 29.5(14) | 48.7(18) | 41.1(15) | −9.2(14) | 4.5(11) | 1.8(13) |
| C17 | 34.6(14) | 34.6(14) | 33.4(13) | −1.3(12) | 1.8(10) | −4.7(12) |
| C13 | 31.9(13) | 38.9(16) | 39.1(14) | 0.3(13) | 0.0(11) | −1.3(12) |
| C26 | 31.2(13) | 30.9(13) | 45.5(16) | −0.7(12) | −1.8(11) | 1.3(11) |
| C2 | 30.7(13) | 47.2(17) | 36.0(14) | −3.7(13) | 2.2(11) | −1.6(13) |
| C8 | 24.1(12) | 45.1(17) | 37.0(14) | −7.5(12) | 3.5(10) | −3.1(12) |
| C1 | 24.4(12) | 46.7(17) | 38.2(15) | −2.2(13) | 3.1(10) | −2.5(12) |
| C24 | 25.2(12) | 36.6(16) | 45.0(16) | −2.3(12) | 0.3(11) | −1.3(11) |
| C18 | 36.8(15) | 39.8(16) | 36.0(14) | −0.6(13) | −2.0(11) | −7.1(13) |
| C22 | 31.7(13) | 32.7(14) | 40.0(15) | −3.5(12) | −0.1(11) | −0.6(12) |
| C3 | 34.0(15) | 44.6(17) | 44.6(16) | 0.8(14) | −1.6(12) | −0.8(13) |
| C25 | 30.8(13) | 27.1(13) | 42.0(15) | −2.2(12) | −0.5(11) | −1.1(11) |
| C31 | 37.1(15) | 37.8(15) | 41.7(15) | 7.4(13) | 3.3(12) | 2.1(12) |
| C6 | 30.4(14) | 51.6(19) | 39.8(15) | −6.2(14) | 2.7(11) | −5.0(13) |
| C23 | 28.7(13) | 37.2(15) | 43.7(16) | 1.4(13) | −3.1(11) | 1.8(12) |
| C30 | 36.7(15) | 41.7(16) | 37.5(14) | 3.1(13) | 2.6(11) | 0.0(13) |
| C27 | 26.8(13) | 34.6(15) | 46.4(16) | −2.8(13) | −0.2(11) | 2.8(11) |
| C10 | 29.3(13) | 40.0(16) | 47.9(16) | −7.4(14) | 0.8(11) | 5.6(13) |
| C16 | 47.9(18) | 35.6(16) | 43.5(16) | −4.9(13) | 5.1(13) | −3.5(14) |
| C15 | 42.7(18) | 35.7(17) | 62(2) | −5.8(15) | 12.1(15) | 6.5(13) |
| C4 | 28.8(14) | 56(2) | 41.9(16) | 8.1(15) | −0.9(11) | −2.0(14) |
| C14 | 28.7(14) | 39.9(17) | 55.8(19) | 4.1(14) | 0.9(12) | 2.1(12) |
| C5 | 30.8(14) | 64(2) | 35.2(14) | −0.8(14) | 2.5(11) | −2.9(14) |
| C34 | 42.7(17) | 52(2) | 45.6(17) | 0.8(15) | 0.3(13) | 8.4(15) |
| C33 | 47.8(18) | 48.9(19) | 51.0(18) | 0.3(16) | −1.6(15) | −9.8(16) |

TABLE 4
Bond Lengths in A for Atropisomer A-2

| Atom | Atom | Length/A |
|---|---|---|
| CI36 | 04 | 1.746(4) |
| F19 | C18 | 1.336(4) |
| F20 | C18 | 1.338(4) |
| F21 | C18 | 1.331(4) |
| 035 | C28 | 1.234(4) |
| N29 | C28 | 1.340(4) |
| N29 | C30 | 1.457(4) |
| N32 | C33 | 1.456(4) |
| N32 | C34 | 1.459(5) |
| N32 | C31 | 1.461(4) |
| N7 | C11 | 1.392(4) |
| N7 | C8 | 1.396(4) |
| N7 | C12 | 1.429(4) |
| 028 | C25 | 1.501(4) |
| C12 | C13 | 1.384(4) |
| C12 | C17 | 1.397(4) |
| C11 | C10 | 1.370(4) |
| C11 | C22 | 1.466(4) |
| 09 | C8 | 1.372(5) |
| 09 | C10 | 1.403(5) |
| C17 | C16 | 1.388(5) |
| C17 | C18 | 1.510(4) |
| C13 | C14 | 1.386(5) |
| C26 | C27 | 1.377(5) |
| 026 | C25 | 1.391(4) |
| 02 | C3 | 1.388(5) |
| 02 | C1 | 1.400(5) |
| 08 | C1 | 1.468(5) |
| C1 | C6 | 1.409(4) |
| 024 | C23 | 1.389(5) |
| C24 | C25 | 1.390(4) |
| C22 | C23 | 1.396(4) |
| C22 | C27 | 1.404(4) |
| C3 | C4 | 1.385(5) |
| C31 | C30 | 1.521(5) |
| C6 | C5 | 1.387(5) |
| C16 | C15 | 1.390(5) |
| C15 | C14 | 1.383(5) |
| C4 | C5 | 1.377(6) |

TABLE 5
Bond Angles in ° for Atropisomer A-2

| Atom | Atom | Atom | Angle/° |
|---|---|---|---|
| C28 | N29 | 030 | 122.6(3) |
| C33 | N32 | 034 | 109.6(3) |
| C33 | N32 | 031 | 110.1(3) |
| C34 | N32 | 031 | 112.1(2) |
| C11 | N7 | 08 | 109.1(3) |
| C11 | N7 | 012 | 126.6(2) |
| 08 | N7 | 012 | 124.2(3) |
| 035 | 028 | N29 | 123.2(3) |
| 035 | 028 | 025 | 120.5(3) |
| N29 | 028 | 025 | 116.2(3) |
| 013 | 012 | 017 | 120.0(3) |
| 013 | 012 | N7 | 118.8(3) |
| 017 | 012 | N7 | 121.2(3) |
| 010 | 011 | N7 | 107.1(3) |
| 010 | 011 | 022 | 128.6(3) |
| N7 | 011 | 022 | 124.3(3) |
| 08 | 09 | 010 | 108.4(3) |
| 016 | 017 | 012 | 119.5(3) |
| 016 | 017 | 018 | 118.7(3) |
| C12 | C17 | C18 | 121.8(3) |
| C12 | C13 | C14 | 120.4(3) |
| C27 | C26 | C25 | 120.5(3) |
| C3 | C2 | C1 | 121.7(3) |
| C9 | C8 | N7 | 106.9(3) |
| C9 | C8 | C1 | 128.1(3) |
| N7 | C8 | C1 | 125.0(3) |
| C2 | C1 | C6 | 117.3(3) |
| C2 | C1 | C8 | 125.0(3) |
| C6 | C1 | C8 | 117.6(3) |
| C23 | C24 | C25 | 120.8(3) |
| F21 | C18 | F19 | 106.0(2) |
| F21 | C18 | F20 | 107.3(3) |
| F19 | C18 | F20 | 105.9(3) |
| F21 | C18 | C17 | 111.8(3) |
| F19 | C18 | C17 | 113.1(2) |
| F20 | C18 | C17 | 112.3(2) |
| C23 | C22 | C27 | 117.9(3) |
| C23 | C22 | C11 | 123.1(3) |
| C27 | C22 | C11 | 119.0(3) |
| C4 | C3 | C2 | 118.8(3) |
| C24 | C25 | C26 | 118.9(3) |
| C24 | C25 | C28 | 121.4(3) |
| C26 | C25 | C28 | 119.6(3) |
| N32 | C31 | C30 | 113.9(3) |
| C5 | C6 | C1 | 121.6(3) |
| C24 | C23 | C22 | 120.6(3) |
| N29 | C30 | C31 | 112.1(2) |
| C26 | C27 | C22 | 121.3(3) |
| C11 | C10 | C9 | 108.5(3) |
| C17 | C16 | C15 | 120.0(3) |
| C14 | C15 | C16 | 120.4(3) |
| C5 | C4 | C3 | 121.8(3) |
| C5 | C4 | CI36 | 119.1(3) |
| C3 | C4 | CI36 | 119.1(3) |
| C15 | C14 | C13 | 119.6(3) |
| C4 | C5 | C6 | 118.9(3) |

TABLE 6
Torsion Angles in ° for Atropisomer A-2

| Atom | Atom | Atom | Atom | Angle/° |
|---|---|---|---|---|
| C30 | N29 | C28 | O35 | −8.5(4) |
| C30 | N29 | C28 | C25 | 170.8(2) |
| C11 | N7 | C12 | C13 | −110.3(3) |
| C8 | N7 | C12 | C13 | 74.5(4) |
| C11 | N7 | C12 | C17 | 71.3(4) |
| C8 | N7 | C12 | C17 | −103.9(3) |
| C8 | N7 | C11 | C10 | 0.3(3) |
| C12 | N7 | C11 | C10 | −175.5(3) |
| C8 | N7 | C11 | C22 | −177.3(3) |
| C12 | N7 | C11 | C22 | 6.8(4) |
| C13 | C12 | C17 | C16 | 2.1(4) |
| N7 | C12 | C17 | C16 | −179.5(3) |
| C13 | C12 | C17 | C18 | −174.1(3) |
| N7 | C12 | C17 | C18 | 4.3(4) |
| C17 | C12 | C13 | C14 | −0.8(5) |
| N7 | C12 | C13 | C14 | −179.2(3) |
| C10 | C9 | C8 | N7 | −0.8(3) |
| C10 | C9 | C8 | C1 | 179.0(3) |
| C11 | N7 | C8 | C9 | 0.3(3) |
| C12 | N7 | C8 | C9 | 176.2(3) |
| C11 | N7 | C8 | C1 | −179.4(3) |
| C12 | N7 | C8 | C1 | −3.5(4) |
| C3 | C2 | C1 | C6 | 0.1(4) |
| C3 | C2 | C1 | C8 | 177.6(3) |
| C9 | C8 | C1 | C2 | −141.6(3) |
| N7 | C8 | C1 | C2 | 38.1(4) |
| C9 | C8 | C1 | C6 | 35.8(4) |
| N7 | C8 | C1 | C6 | −144.5(3) |
| C16 | C17 | C18 | F21 | −11.9(4) |
| C12 | C17 | C18 | F21 | 164.2(3) |
| C16 | C17 | C18 | F19 | 107.6(3) |
| C12 | C17 | C18 | F19 | −76.2(4) |
| C16 | C17 | C18 | F20 | −132.6(3) |
| C12 | C17 | C18 | F20 | 43.6(4) |
| C10 | C11 | C22 | C23 | −139.7(3) |
| N7 | C11 | C22 | C23 | 37.4(5) |
| C10 | C11 | C22 | C27 | 38.3(5) |
| N7 | C11 | C22 | C27 | −144.6(3) |
| C1 | C2 | C3 | C4 | 0.2(5) |
| C23 | C24 | C25 | C26 | 0.2(5) |
| C23 | C24 | C25 | C28 | −175.1(3) |
| C27 | C26 | C25 | C24 | 1.3(5) |
| C27 | C26 | C25 | C28 | 176.7(3) |
| O35 | C28 | C25 | C24 | 144.1(3) |
| N29 | C28 | C25 | C24 | −35.3(4) |
| O35 | C28 | C25 | C26 | −31.2(4) |
| N29 | C28 | C25 | C26 | 149.5(3) |
| C33 | N32 | C31 | C30 | 169.7(3) |
| C34 | N32 | C31 | C30 | −68.1(4) |
| C2 | C1 | C6 | C5 | 0.0(4) |
| C8 | C1 | C6 | C5 | −177.6(3) |
| C25 | C24 | C23 | C22 | −1.3(5) |
| C27 | C22 | C23 | C24 | 0.9(5) |
| C11 | C22 | C23 | C24 | 179.0(3) |
| C28 | N29 | C30 | C31 | −80.9(4) |
| N32 | C31 | C30 | N29 | −55.5(4) |
| C25 | C26 | C27 | C22 | −1.7(5) |
| C23 | C22 | C27 | C26 | 0.6(5) |
| C11 | C22 | C27 | C26 | −177.6(3) |
| N7 | C11 | C10 | C9 | −0.8(3) |
| C22 | C11 | C10 | C9 | 176.8(3) |
| C8 | C9 | C10 | C11 | 0.9(3) |
| C12 | C17 | C16 | C15 | −1.7(5) |
| C18 | C17 | C16 | C15 | 174.6(3) |
| C17 | C16 | C15 | C14 | −0.1(5) |
| C2 | C3 | C4 | C5 | −0.7(5) |
| C2 | C3 | C4 | CI36 | 177.7(2) |
| C16 | C15 | C14 | C13 | 1.5(5) |
| C12 | C13 | C14 | C15 | −1.0(5) |
| C3 | C4 | C5 | C6 | 0.8(5) |
| CI36 | C4 | C5 | C6 | −177.5(2) |
| C1 | C6 | C5 | C4 | −0.5(4) |

TABLE 7
Hydrogen Fractional Atomic Coordinates (×10⁴) and Equivalent
Isotropic Displacement Parameters (Å² × 10³) for Atropisomer
A-2. U_{eq} is defined as ⅓ of the trace of the orthogonalised Uᵢⱼ.

| Atom | x | y | z | U_{eq} |
|---|---|---|---|---|
| H29 | 4556.28 | 2597.1 | 9613.5 | 44 |
| H9 | 9373.45 | 3631.45 | 4279.86 | 48 |
| H13 | 5075.26 | 5792.63 | 5065.04 | 44 |
| H26 | 8091.21 | 838.52 | 8764.29 | 43 |
| H2 | 7844.85 | 8248.03 | 5190.71 | 46 |
| H24 | 4521.78 | 2695.77 | 8048.38 | 43 |
| H3 | 7771.85 | 10303.2 | 4130.77 | 49 |
| H31A | 3309.1 | −1006.73 | 9802.84 | 47 |
| H31B | 2411.76 | −508.61 | 10623.33 | 47 |
| H6 | 8091.06 | 5215.49 | 3052.71 | 49 |
| H23 | 5315.8 | 3715.16 | 6693.61 | 44 |
| H30A | 3620.15 | 1767.17 | 10885.52 | 46 |
| H30B | 4611.45 | 327.4 | 10937.24 | 46 |
| H27 | 8912.55 | 1941.11 | 7453.43 | 43 |
| H10 | 9042.29 | 1887.07 | 5647.28 | 47 |
| H16 | 6460.32 | 8712.19 | 7693.92 | 51 |
| H15 | 4381.36 | 9123.95 | 6944.76 | 56 |
| H14 | 3703.76 | 7704.45 | 5613.91 | 50 |
| H5 | 8029.04 | 7271.37 | 1993.52 | 52 |
| H34A | 665.96 | 1339.38 | 10310.64 | 71 |
| H34B | 634.49 | 2342.88 | 9371.1 | 71 |
| H34C | 1786.66 | 2599.59 | 10171.65 | 71 |
| H33A | 1667.2 | −1246.13 | 8637.31 | 74 |
| H33B | 539.51 | 36.17 | 8485.32 | 74 |
| H33C | 622.37 | −1010.97 | 9408.31 | 74 |

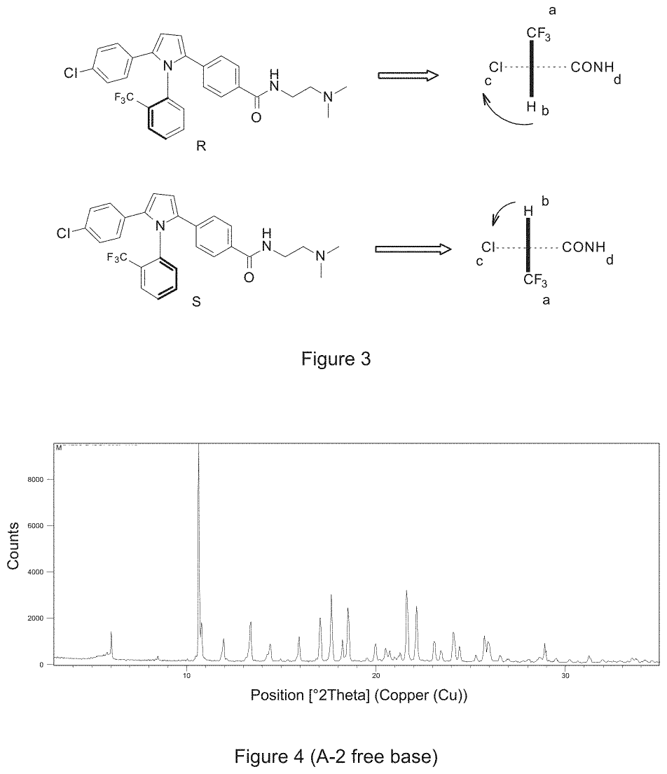

On the basis of the data set out below, atropisomer A-2 is believed to have the R configuration as shown in FIGS. 2 and 3 and can therefore be named as (R)-4-[5-(4-chlorophenyl)-1-[2-(trifluoromethyl)phenyl]pyrrol-2-yl]-N-[2 (dimethylamino)-ethyl]benzamide.

### Example 4

Preparation of Atropisomers A-3 and A-4

Atropisomers A-3 and A-4 were prepared by following Synthetic Route B, as shown below.

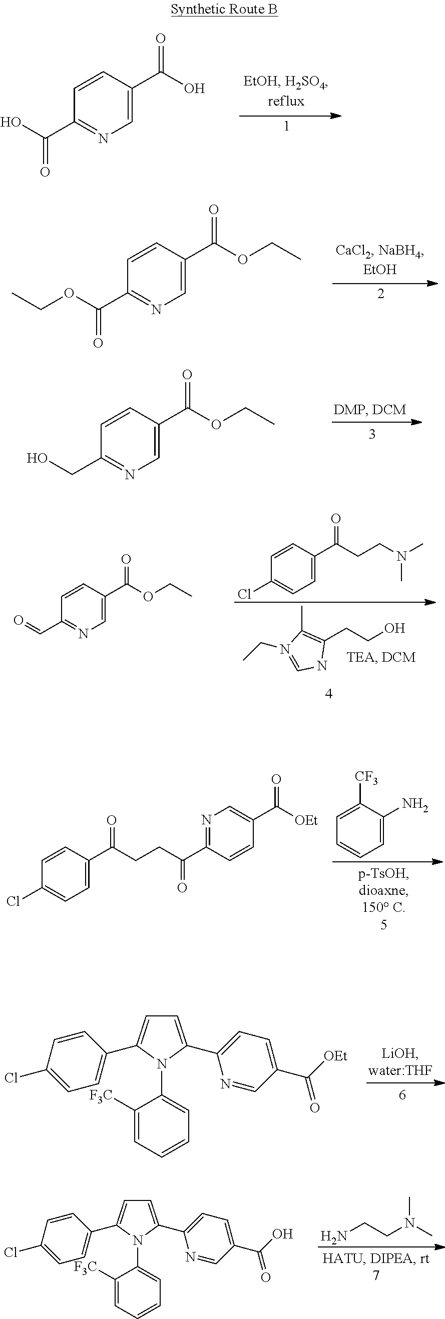

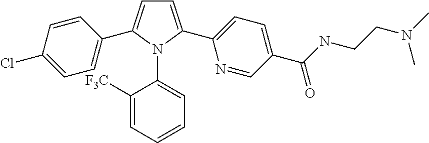 [SMILES: CN(C)CCNC(=O)c1ccc(-c2ccc(-c3ccc(Cl)cc3)n2-c2ccccc2C(F)(F)F)nc1]

### Step 1: Diethyl pyridine-2,5-dicarboxylate

To a suspension of 2, 5-pyridinedicarboxylic acid (20 g, 120 mmol) in absolute EtOH (120 mL) was added conc. H₂SO₄ (25.6 mL, 0.048 mmol) dropwise over a period of 30 min. The resulting reaction mixture was refluxed for 48 h. The reaction mixture was concentrated, and the resulting residue basified to pH 8 (sat. aq. NaHCO₃). The resulting aqueous layer was extracted with EtOAC (4×200 mL). The combined organic layers were washed with brine, washed, dried (Na₂SO₄) and concentrated. Four other 20 g batches were reacted in parallel and the resulting crude material from each reaction was combined and purified by column chromatography on silica gel (60-120 mesh) eluting with 5% EtOAC/hexane to afford the title compound (65 g, 291 mmol, 49%).

### Step 2: Ethyl 6-(hydroxymethyl)pyridine-3-carboxylate

To a cooled (ice-bath) solution of diethyl pyridine-2, 5-dicarboxylate (10 g, 45 mmol) in a mixture of absolute EtOH (40 mL) and THF (3.5 mL) under nitrogen were added NaBH₄ (4.26 g, 112 mmol) and anhydrous CaCl₂ (7.86 g, 71 mmol) portion wise over 30 min. The resulting reaction mixture was stirred at 0° C. for 5 h. The reaction mixture was poured in sat. aq. NH₄Cl (150 mL) and extracted with EtOAc (4×150 mL). The combined organic extracts were dried Na₂SO₄) and concentrated. Six other 10 g batches and one 5 g batch were reacted in parallel and the resulting crude material from each reaction was combined and purified by column chromatography with silica gel (60-120 mesh) eluting with 20% EtOAc/hexane to afford the title compound (55 g, 320 mmol, 100%).

### Step 3: Ethyl 6-formylpyridine-3-carboxylate

To a cooled (ice-bath) solution of ethyl 6-(hydroxymethyl)pyridine-3-carboxylate (30 g, 166 mmol) in DCM (360 mL) under nitrogen was added DMP (84.32 g, 199 mmol) portion wise over 20 min. The reaction was stirred at rt for 3 h. The reaction mixture was poured into ice-cold water (1.5 L) and the resulting mixture basified to ˜pH 8 (sat. aq. NaHCO₃) and extracted with EtOAc (4×1000 mL). The combined organic layers were washed with brine, dried (Na₂SO₄) and concentrated. The resulting residue was purified by column chromatography with silica gel (60-120 mesh) eluting with 12% EtOAc/hexane to afford the title compound (19 g, 106 mmol, 33%).

### Step 4: Ethyl 6-[4-(4-chlorphenyl)-4-oxo-butanoyl]pyridine-3-carboxylate

To a stirred solution of intermediate A (1.17 g, 5.6 mmol) and TEA (1.56 mL, 11.2 mmol) in 1,2-dimethoxyethane (10 mL) were added ethyl 6-formylpyridine-3-carboxylate (1 g, 5.6 mmol) and 3-ethyl-5-(2-hydroxyethyl)-4-methylthiazol-3-ium bromide (0.28 g, 11.2 mmol) at room temperature. The resulting solution was heated at 80-90° C. for 5 h. The reaction was diluted with ice-cold water (400 mL) and extracted with EtOAc (3×200 mL). The combined organic layers were dried (Na₂SO₄) and concentrated. The resulting residue was purified by column chromatography with silica gel (60-120 mesh) eluting with 8% EtOAc/hexane to afford the title compound (5.5 g, 15.9 mmol, 17%).

### Step 5: Ethyl 6-[5-(4-chlorophenyl)-1-[2-(trifluoromethyl)phenyl]pyrrol-2-yl]pyridine-3-carboxylate

To a solution of ethyl 6-[4-(4-chlorophenyl)-4-oxo-butanoyl]pyridine-3-carboxylate (2.5 g, 7.2 mmol) in 1,4-dioxane (25 mL) were added 2-aminobenzotrifluoride (3.5 g, 21.7 mmol) and PTSA (0.14 g, 0.72 mmol) at room temperature. The resulting solution was heated at 150° C. for 48 h. The reaction mixture was concentrated and purified by column chromatography with silica gel (60-120 mesh) eluting with 6% EtOAc/hexane to afford the title compound (2.5 g, 5.3 mmol, 64%).

### Step 6: 6-[5-(4-chlorophenyl)-1-[2-(trifluoromethyl)phenyl]pyrrol-2-yl]pyridine-3-carboxylic acid

To a solution of ethyl 6-[5-(4-chlorophenyl)-1-[2-(trifluoromethyl)phenyl]pyrrol-2-yl]pyridine-3-carboxylate (2.2 g, 4.7 mmol) in mixture of THF (10 mL) and water (10 mL) at room temperature was added UOH (0.59 g, 14 mmol). The resulting solution was stirred at 80° C. for 16 h. The reaction mixture was concentrated, diluted with water (150 mL) and extracted with EtOAc (4×150 mL). The combined organic extracts were dried (Na₂SO₄) and concentrated. The resulting material was triturated with n-pentane (15 mL) and Et₂O (15 mL) to afford the title compound (2 g, 4.5 mmol, 97%).

### Step 7: 4-[5-(4-chlorophenyl)-1-[2-(trifluoromethyl)phenyl]pyrrol-2-yl]-N-[2-(dimethylamino)ethyl]benzamide

To a solution of 6-[5-(4-chlorophenyl)-1-[2-(trifluoromethyl)phenyl]pyrrol-2-yl]pyridine-3-carboxylic acid (2.8 g, 6.33 mol) in DMF (20 mL) was added HATU (7.22 g, 19 mol) and the reaction mixture was stirred at room temperature for 20 min. Unsym-N, N-dimethyl ethylenediamine (1.11 g, 12.7 mol) and DIPEA (3.31 mL, 19 mol) were added and the reaction mixture was stirred at room temperature for 4 h. The reaction mixture was diluted with ice-cold water (200 mL) and extracted with EtOAc (4×100 mL). The combined organic extracts were dried (Na₂SO₄) and concentrated. The resulting residue was purified by column chromatography with silica gel (60-120 mesh) eluting with 30% EtOAc/hexane) to afford the title compound (2.4 g, 4.7 mmol, 74%).

### Step 8: Separation of Atropisomers A-3 and A-4

The atropisomers of 6-[5-(4-chlorophenyl)-1-[2-(trifluoromethyl)phenyl]pyrrol-2-yl]-N-[2-(dimethylamino)ethyl]pyridine-3-carboxamide may be resolved by chiral HPLC using preparative chiral HPLC method 2.

Two peaks were isolated:
Peak 1: Atropisomer A-3, 6-[5-(4-chlorophenyl)-1-[2-(trifluoromethyl)phenyl]pyrrol-2-yl]-N-[2-(dimethylamino)ethyl]pyridine-3-carboxamide-atropisomer 1 (70 mg, 0.14 mmol, 355%), brown solid.
Peak 2: Atropisomer A-4, 6-[5-(4-chlorophenyl)-1-[2-(trifluoromethyl)phenyl]pyrrol-2-yl]-N-[2-(dimethylamino)ethyl]pyridine-3-carboxamide-atropisomer 2 (75 mg, 0.15 mmol, 38%), brown solid.

Both peaks were purified further to remove aliphatic impurities:

Peak 1: (A-3) (57 mg, 0.11 mmol) was diluted with HPLC grade water (25 mL) followed by sonication for 10 min and extraction with EtOAc (3×20 mL). The combined organic extracts were dried (Na₂SO₄), filtered, concentrated and lyophilised to afford atropisomer A-3 (56 mg, 0.11 mmol, 98%, >99% ee).

¹H NMR (DMSO-d₆) δ 8.45-8.43 (m, 2H), 8.01 (d, J=6.8 Hz, 1H), 7.74-7.68 (m, 2H), 7.65-7.60 (m, 3H), 7.25 (d, J=8.4 Hz, 2H), 7.11-7.04 (m, 3H), 6.60 (d, J=4 Hz, 1H), 3.32 (m, 2H, obscured by residual water peak), 2.30 (m, 2H, obscured by residual solvent peak), 2.19 (s, 6H). LCMS (Method 1)—RT 2.41, MH+ 513.4

Peak 2: (A-4): (60 mg, 0.117 mmol) was diluted with HPLC grade water (25 mL) followed by sonication for 10 min and extraction with EtOAc (3×20 mL). The combined organic extracts were dried (Na₂SO₄), filtered, concentrated and lyophilised to afford Example A-4 (60 mg, 0.12 mmol, 99%, 95% ee).

¹H NMR (DMSO-d₆) δ 8.47-8.43 (m, 2H), 8.02 (d, J=7.2 Hz, 1H), 7.74-7.68 (m, 2H), 7.65-7.60 (m, 3H), 7.25 (d, J=8.4 Hz, 2H), 7.11-7.04 (m, 3H), 6.60 (d, J=4 Hz, 1H), 3.32 (m, 2H, obscured by residual water peak), 2.30 (m, 2H, obscured by residual solvent pea), 2.20 (s, 6H). LCMS (Method 1)—RT 2.41, MH+ 513.4

Chiral Analysis

Analysis of the chiral properties of the Atropisomers A-3 and A-4 was carried out by measuring their optical rotations and their retention times obtained by chiral HPLC using the methods described above to give the results shown in the table below.

| | Chiral | Chiral | Specific |
|---|---|---|---|
| | HPLC RT | HPLC | Optical |
| Example | (min) | Method | Rotation |
| A-3 | 10.400 | 2 | +9.92° |
| A-4 | 12.090 | 2 | −4.89° |

### Example 5

### Preparation of Atropisomers A-5 and A-6: N-[2-(dimethylamino)ethyl]-6-[5-(4-fluorophenyl)-1-[2-(trifluoromethyl)phenyl]pyrrol-2-yl]pyridine-3-carboxamide

Atropisomers A-5 and A-6 were prepared as a racemic mixture using the same method as described above in Example 4 for atropisomers A-3 and A-4 with the following exceptions: (a) Intermediate B (3.23 g, 16.58 mmol) was used in step 4 and 3-benzyl-5-(2-hydroxyethyl)-4-methylthiazol-3-ium bromide (0.678 g, 2.51 mmol) and purification was carried out using 10% EtOAc/hexane as eluent (b) step 5 purification used 1.3% EtOAc/hexane as eluent (c) MeOH was used instead of THF in step 6 and purification was trituration with Et₂O (d) In step 7 the isolated residue was purified by chromatography with basic alumina gel eluting with DCM to afford the title compound (0.16 g, 0.32 mmol, 55%) (e) Purification by preparative HPLC method 1 afforded the title compound (61 mg, 0.12 mmol, 38%) (racemic mixture of atropisomers) as its hydrochloride salt, a light yellow solid.

¹H NMR (DMSO-d₆) δ 10.09 (bs, 1H), 8.85 (m, 1H), 8.49 (s, 1H), 8.11 (d, J=8.0 Hz, 1H), 7.73-7.70 (m, 2H), 7.69-7.61 (m, 3H), 7.13-7.10 (m, 3H), 7.06-7.02 (m, 2H), 6.56 (d, J=4.0 Hz, 1H), 3.56 (m, 2H), 3.20 (m, 2H), 2.76 (d, J=4.4 Hz, 6H).

LCMS (Method 2)—RT 5.06, MH+ 497.2

Chiral HPLC analysis with chiral HPLC method 3 indicated a mixture of atropisomers, RT peak 1, 9.95 min, 49.8% area (Atropisomer A-5) and peak 2, 11.52 min, 50.2% area (Atropisomer A-6).

### Example 6

### Preparation of Atropisomers A-7 and A-8 of 6-[5-(4-cyanophenyl)-1-[2-(trifluoromethyl)phenyl]pyrrol-2-yl]-N-[2-(dimethylamino)ethyl]pyridine-3-carboxamide

Atropisomers A-7 and A-8 were prepared as a racemic mixture using the same method as described above in Example 4 for atropisomers A-3 and A-4 with the following exceptions: (a) Intermediate C (0.28 g, 1.39 mmol) was used in step 4 and 3-benzyl-5-(2-hydroxyethyl)-4-methylthiazol-3-ium bromide (0.04 g, 0.14 mmol) and purification was carried out using 10% EtOAc/hexane as eluent (b) step 5 purification used 7% EtOAc/hexane as eluent (c) In step 7 the isolated residue was purified by chromatography with basic alumina gel eluting with 10% EtOAc/hexane to afford the title compound (0.13 g, 0.25 mmol, 75%) (d) Purification by preparative HPLC method 2 afforded the title compound (54 mg, 0.11 mmol, 36%) as its hydrochloride salt, a light yellow solid.

¹H NMR (DMSO-d₆) δ 9.83 (brs, 1H), 8.80 (t, J=5.2 HZ, 1H), 8.50 (d, J=1.6 Hz, 1H), 8.11 (dd, J=8.4, 2.0 Hz, 1H), 7.79-7.64 (m, 6H), 7.32-7.07 (m, 4H), 6.83 (d, J=4.0 Hz, 1H), 3.56-3.46 (m, 2H), 3.22-3.18 (m, 2H), 2.78 (d, J=4.8 Hz, 6H).

LCMS (Method 1)—RT 2.05, MH+ 504.1

Chiral HPLC analysis with chiral HPLC method 4 indicated a mixture of atropisomers, RT peak 1, 8.82 min, 50.2% area (Example A-7) and peak 2, 10.10 min, 49.8% area (Example A-8).

### Example 7

Preoaration of Compounds B-2 to B-107

Further examples of atropisomer compounds of the present invention can be prepared by preparing racemic mixtures of the compounds shown in the table below, and then separating the individual atropisomers using the chiral HPLC methods described above or methods similar thereto. In the table, the Compound numbers given correspond to the Example numbers in our earlier International patent application WO2018/197714 but with the prefix B- added. Thus, Compound B-2 corresponds to Example 2 in WO2018/197714, Compound B-3 corresponds to Example 3 in WO2018/197714 and so on. The NMR, LCMS and other characterising data for the racemic compounds and their biological activity data are as given in WO02018/197714.

| | |
|---|---|
| 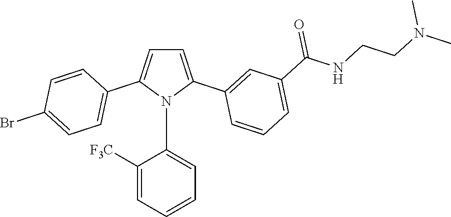 [SMILES: CN(C)CCNC(=O)c1cccc(-c2ccc(-c3ccc(Br)cc3)n2-c2ccccc2C(F)(F)F)c1] | Compound B-2 |
| 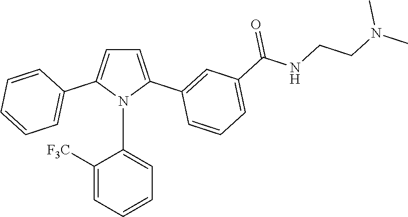 [SMILES: CN(C)CCNC(=O)c1cccc(-c2ccc(-c3ccccc3)n2-c2ccccc2C(F)(F)F)c1] | Compound B-3 |
| 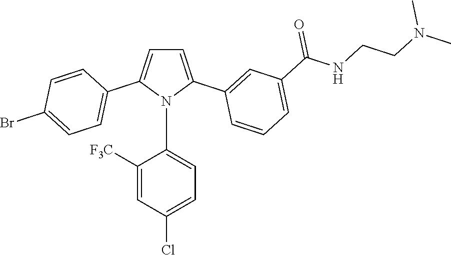 [SMILES: CN(C)CCNC(=O)c1cccc(-c2ccc(-c3ccc(Br)cc3)n2-c2ccc(Cl)cc2C(F)(F)F)c1] | Compound B-4 |
| 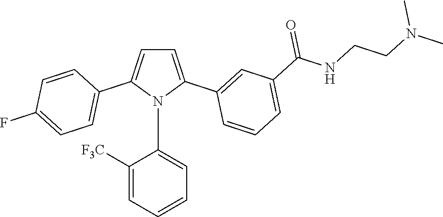 [SMILES: CN(C)CCNC(=O)c1cccc(-c2ccc(-c3ccc(F)cc3)n2-c2ccccc2C(F)(F)F)c1] | Compound B-5 |
| 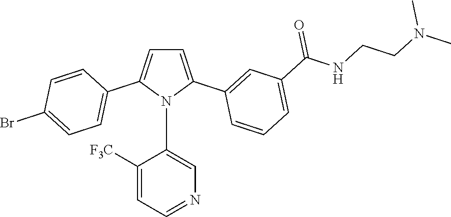 [SMILES: CN(C)CCNC(=O)c1cccc(-c2ccc(-c3ccc(Br)cc3)n2-c2cnccc2C(F)(F)F)c1] | Compound B-6 |
| 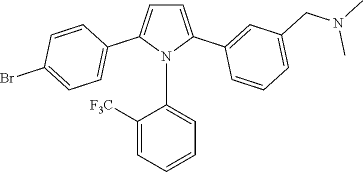 [SMILES: CN(C)Cc1cccc(-c2ccc(-c3ccc(Br)cc3)n2-c2ccccc2C(F)(F)F)c1] | Compound B-7 |
| 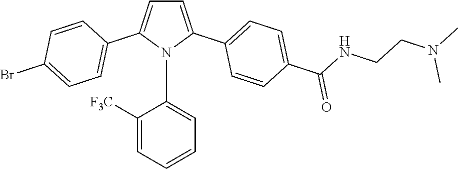 [SMILES: CN(C)CCNC(=O)c1ccc(-c2ccc(-c3ccc(Br)cc3)n2-c2ccccc2C(F)(F)F)cc1] | Compound B-9 |
| 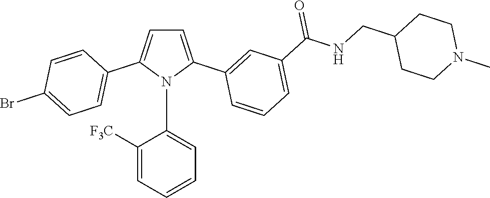 [SMILES: CN1CCC(CNC(=O)c2cccc(-c3ccc(-c4ccc(Br)cc4)n3-c3ccccc3C(F)(F)F)c2)CC1] | Compound B-10 |
| 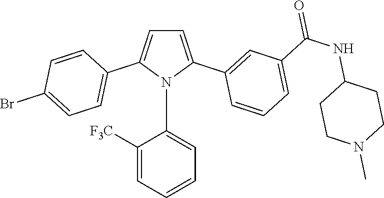 [SMILES: CN1CCC(NC(=O)c2cccc(-c3ccc(-c4ccc(Br)cc4)n3-c3ccccc3C(F)(F)F)c2)CC1] | Compound B-11 |
| 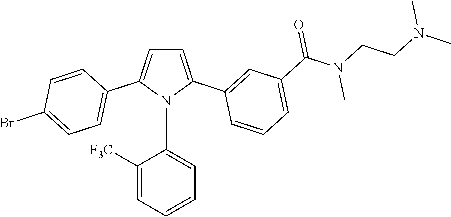 [SMILES: CN(C)CCN(C)C(=O)c1cccc(-c2ccc(-c3ccc(Br)cc3)n2-c2ccccc2C(F)(F)F)c1] | Compound B-12 |
| 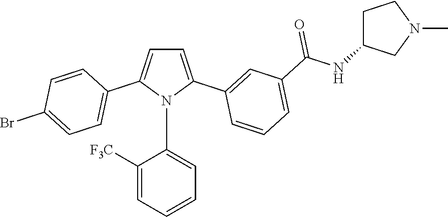 [SMILES: CN1CC[C@@H](NC(=O)c2cccc(-c3ccc(-c4ccc(Br)cc4)n3-c3ccccc3C(F)(F)F)c2)C1] | Compound B-13 |
| 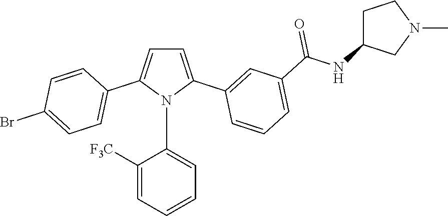 [SMILES: CN1CC[C@H](NC(=O)c2cccc(-c3ccc(-c4ccc(Br)cc4)n3-c3ccccc3C(F)(F)F)c2)C1] | Compound B-14 |
| 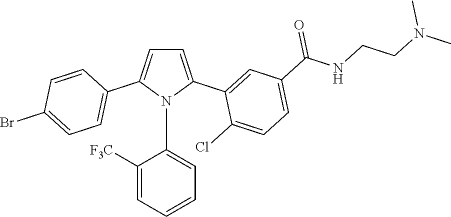 [SMILES: CN(C)CCNC(=O)c1ccc(Cl)c(-c2ccc(-c3ccc(Br)cc3)n2-c2ccccc2C(F)(F)F)c1] | Compound B-15 |
| 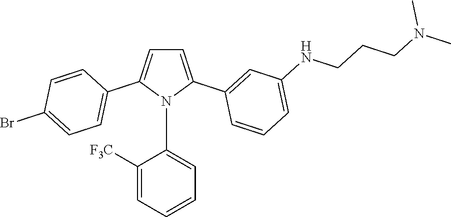 [SMILES: CN(C)CCCNc1cccc(-c2ccc(-c3ccc(Br)cc3)n2-c2ccccc2C(F)(F)F)c1] | Compound B-16 |
| 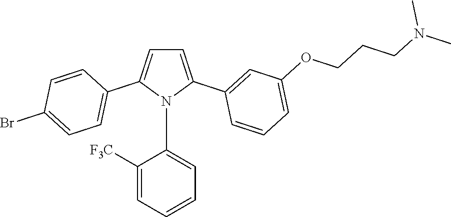 [SMILES: CN(C)CCCOc1cccc(-c2ccc(-c3ccc(Br)cc3)n2-c2ccccc2C(F)(F)F)c1] | Compound B-17 |
| 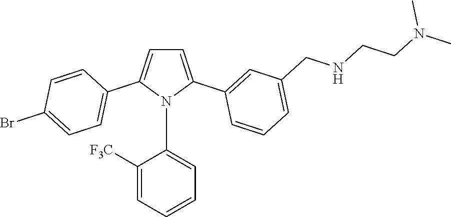 [SMILES: CN(C)CCNCc1cccc(-c2ccc(-c3ccc(Br)cc3)n2-c2ccccc2C(F)(F)F)c1] | Compound B-18 |
| 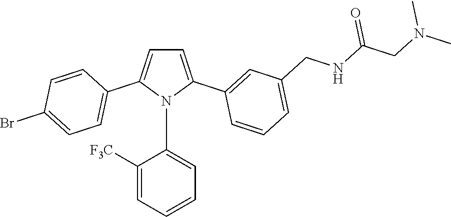 [SMILES: CN(C)CC(=O)NCc1cccc(-c2ccc(-c3ccc(Br)cc3)n2-c2ccccc2C(F)(F)F)c1] | Compound B-21 |
| 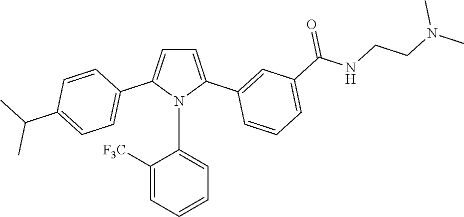 [SMILES: CC(C)c1ccc(-c2ccc(-c3cccc(C(=O)NCCN(C)C)c3)n2-c2ccccc2C(F)(F)F)cc1] | Compound B-22 |
| 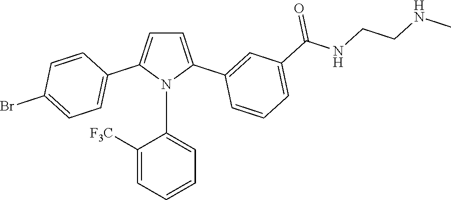 [SMILES: CNCCNC(=O)c1cccc(-c2ccc(-c3ccc(Br)cc3)n2-c2ccccc2C(F)(F)F)c1] | Compound B-23 |
| 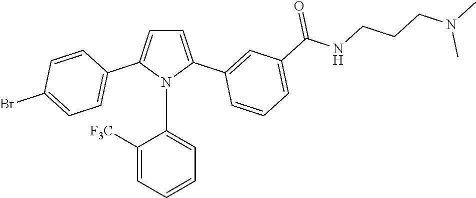 [SMILES: CN(C)CCCNC(=O)c1cccc(-c2ccc(-c3ccc(Br)cc3)n2-c2ccccc2C(F)(F)F)c1] | Compound B-24 |
| 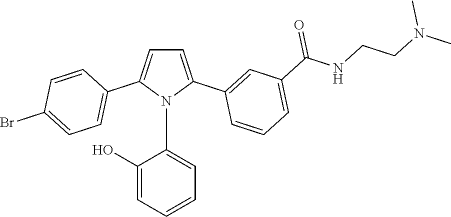 [SMILES: CN(C)CCNC(=O)c1cccc(-c2ccc(-c3ccc(Br)cc3)n2-c2ccccc2O)c1] | Compound B-25 |
| 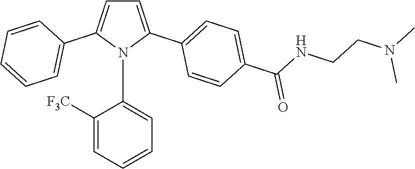 [SMILES: CN(C)CCNC(=O)c1ccc(-c2ccc(-c3ccccc3)n2-c2ccccc2C(F)(F)F)cc1] | Compound B-26 |
| 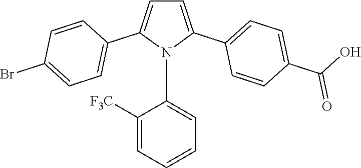 [SMILES: O=C(O)c1ccc(-c2ccc(-c3ccc(Br)cc3)n2-c2ccccc2C(F)(F)F)cc1] | Compound B-27 |
| 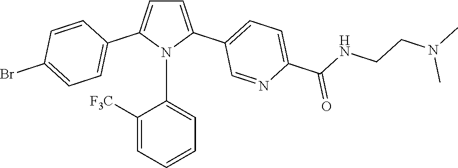 [SMILES: CN(C)CCNC(=O)c1ccc(-c2ccc(-c3ccc(Br)cc3)n2-c2ccccc2C(F)(F)F)cn1] | Compound B-28 |
| 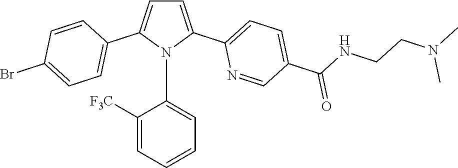 [SMILES: CN(C)CCNC(=O)c1ccc(-c2ccc(-c3ccc(Br)cc3)n2-c2ccccc2C(F)(F)F)nc1] | Compound B-29 |
| 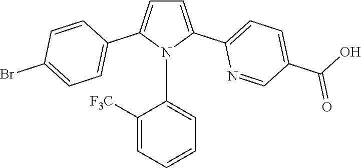 [SMILES: O=C(O)c1ccc(-c2ccc(-c3ccc(Br)cc3)n2-c2ccccc2C(F)(F)F)nc1] | Compound B-30 |
| 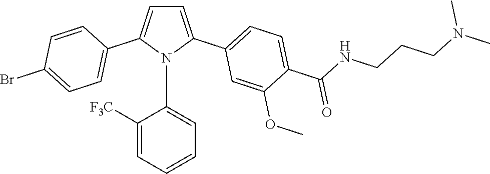 [SMILES: COc1cc(-c2ccc(-c3ccc(Br)cc3)n2-c2ccccc2C(F)(F)F)ccc1C(=O)NCCCN(C)C] | Compound B-31 |
| 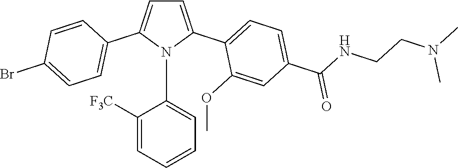 [SMILES: COc1cc(C(=O)NCCN(C)C)ccc1-c1ccc(-c2ccc(Br)cc2)n1-c1ccccc1C(F)(F)F] | Compound B-32 |
| 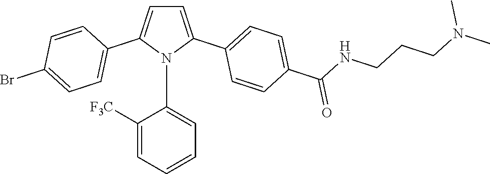 [SMILES: CN(C)CCCNC(=O)c1ccc(-c2ccc(-c3ccc(Br)cc3)n2-c2ccccc2C(F)(F)F)cc1] | Compound B-34 |
| 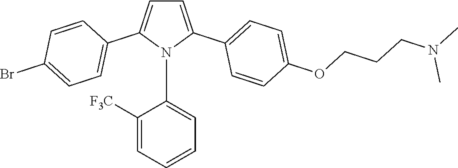 [SMILES: CN(C)CCCOc1ccc(-c2ccc(-c3ccc(Br)cc3)n2-c2ccccc2C(F)(F)F)cc1] | Compound B-35 |
| 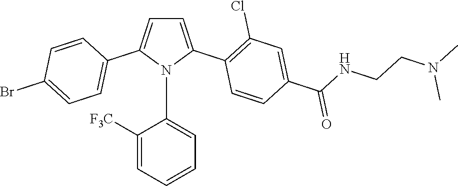 [SMILES: CN(C)CCNC(=O)c1ccc(-c2ccc(-c3ccc(Br)cc3)n2-c2ccccc2C(F)(F)F)c(Cl)c1] | Compound B-36 |
| 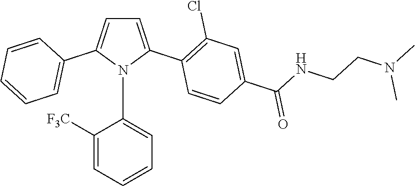 [SMILES: CN(C)CCNC(=O)c1ccc(-c2ccc(-c3ccccc3)n2-c2ccccc2C(F)(F)F)c(Cl)c1] | Compound B-37 |
| 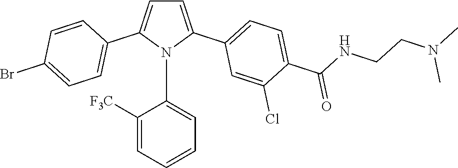 [SMILES: CN(C)CCNC(=O)c1ccc(-c2ccc(-c3ccc(Br)cc3)n2-c2ccccc2C(F)(F)F)cc1Cl] | Compound B-38 |
| 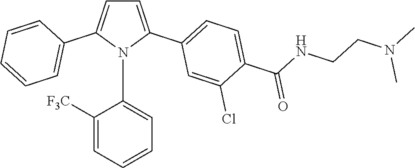 [SMILES: CN(C)CCNC(=O)c1ccc(-c2ccc(-c3ccccc3)n2-c2ccccc2C(F)(F)F)cc1Cl] | Compound B-39 |
| 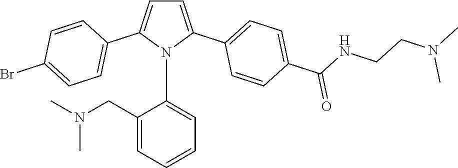 [SMILES: CN(C)CCNC(=O)c1ccc(-c2ccc(-c3ccc(Br)cc3)n2-c2ccccc2CN(C)C)cc1] | Compound B-40 |
| 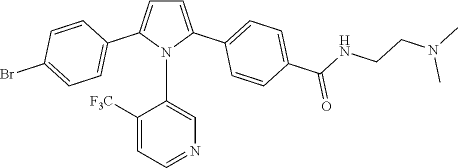 [SMILES: CN(C)CCNC(=O)c1ccc(-c2ccc(-c3ccc(Br)cc3)n2-c2cnccc2C(F)(F)F)cc1] | Compound B-41 |
| 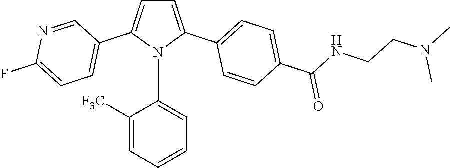 [SMILES: CN(C)CCNC(=O)c1ccc(-c2ccc(-c3ccc(F)nc3)n2-c2ccccc2C(F)(F)F)cc1] | Compound B-42 |
| 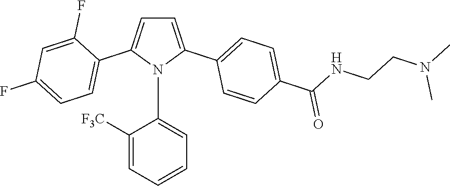 [SMILES: CN(C)CCNC(=O)c1ccc(-c2ccc(-c3ccc(F)cc3F)n2-c2ccccc2C(F)(F)F)cc1] | Compound B-43 |
| 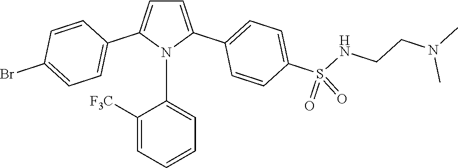 [SMILES: CN(C)CCNS(=O)(=O)c1ccc(-c2ccc(-c3ccc(Br)cc3)n2-c2ccccc2C(F)(F)F)cc1] | Compound B-44 |
| 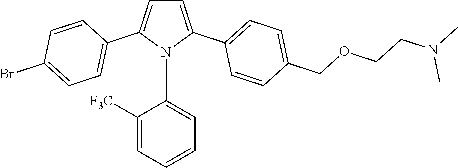 [SMILES: CN(C)CCOCc1ccc(-c2ccc(-c3ccc(Br)cc3)n2-c2ccccc2C(F)(F)F)cc1] | Compound B-45 |
| 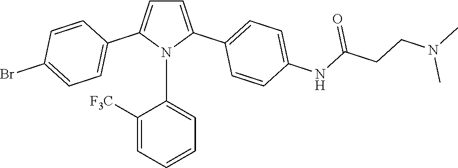 [SMILES: CN(C)CCC(=O)Nc1ccc(-c2ccc(-c3ccc(Br)cc3)n2-c2ccccc2C(F)(F)F)cc1] | Compound B-46 |
| 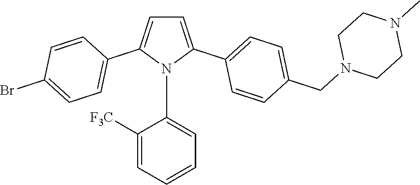 [SMILES: CN1CCN(Cc2ccc(-c3ccc(-c4ccc(Br)cc4)n3-c3ccccc3C(F)(F)F)cc2)CC1] | Compound B-48 |
| 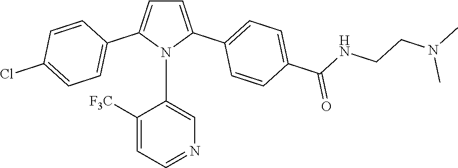 [SMILES: CN(C)CCNC(=O)c1ccc(-c2ccc(-c3ccc(Cl)cc3)n2-c2cnccc2C(F)(F)F)cc1] | Compound B-49 |
| 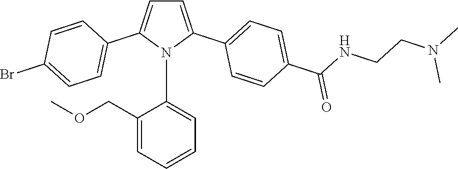 [SMILES: COCc1ccccc1-n1c(-c2ccc(Br)cc2)ccc1-c1ccc(C(=O)NCCN(C)C)cc1] | Compound B-50 |
| 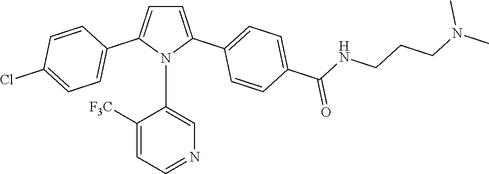 [SMILES: CN(C)CCCNC(=O)c1ccc(-c2ccc(-c3ccc(Cl)cc3)n2-c2cnccc2C(F)(F)F)cc1] | Compound B-51 |
| 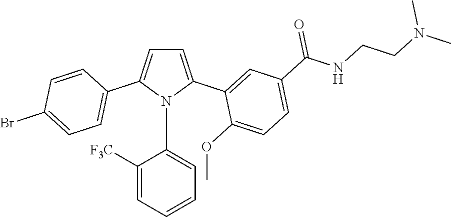 [SMILES: COc1ccc(C(=O)NCCN(C)C)cc1-c1ccc(-c2ccc(Br)cc2)n1-c1ccccc1C(F)(F)F] | Compound B-52 |
| 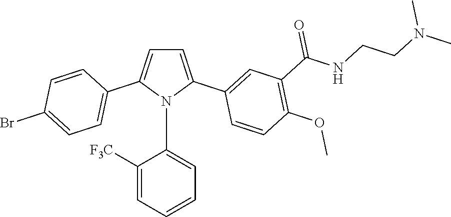 [SMILES: COc1ccc(-c2ccc(-c3ccc(Br)cc3)n2-c2ccccc2C(F)(F)F)cc1C(=O)NCCN(C)C] | Compound B-53 |
| 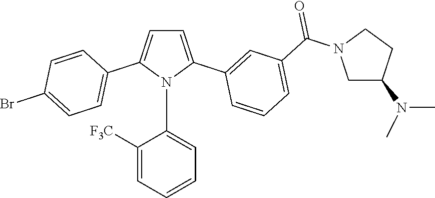 [SMILES: CN(C)[C@@H]1CCN(C(=O)c2cccc(-c3ccc(-c4ccc(Br)cc4)n3-c3ccccc3C(F)(F)F)c2)C1] | Compound B-54 |
| 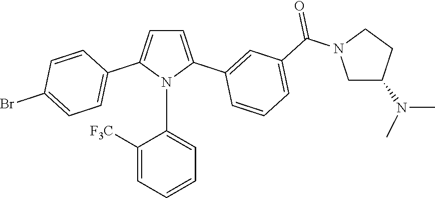 [SMILES: CN(C)[C@H]1CCN(C(=O)c2cccc(-c3ccc(-c4ccc(Br)cc4)n3-c3ccccc3C(F)(F)F)c2)C1] | Compound B-55 |
| 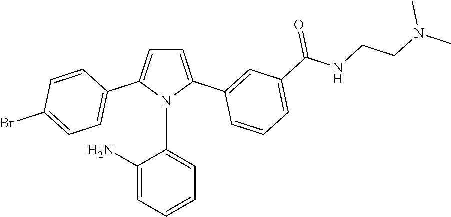 [SMILES: CN(C)CCNC(=O)c1cccc(-c2ccc(-c3ccc(Br)cc3)n2-c2ccccc2N)c1] | Compound B-56 |
| 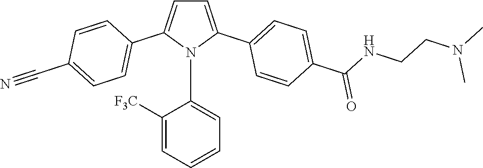 [SMILES: CN(C)CCNC(=O)c1ccc(-c2ccc(-c3ccc(C#N)cc3)n2-c2ccccc2C(F)(F)F)cc1] | Compound B-57 |
| 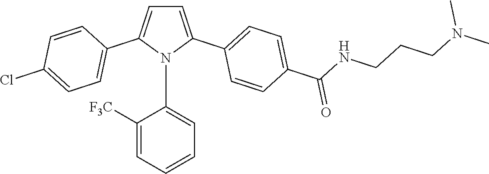 [SMILES: CN(C)CCCNC(=O)c1ccc(-c2ccc(-c3ccc(Cl)cc3)n2-c2ccccc2C(F)(F)F)cc1] | Compound B-58 |
| 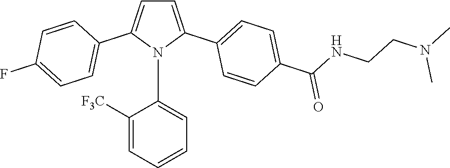 [SMILES: CN(C)CCNC(=O)c1ccc(-c2ccc(-c3ccc(F)cc3)n2-c2ccccc2C(F)(F)F)cc1] | Compound B-59 |
| 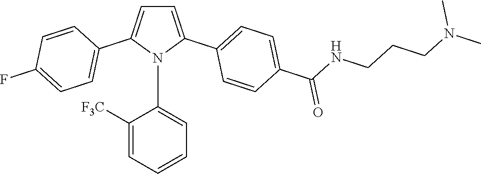 [SMILES: CN(C)CCCNC(=O)c1ccc(-c2ccc(-c3ccc(F)cc3)n2-c2ccccc2C(F)(F)F)cc1] | Compound B-60 |
| 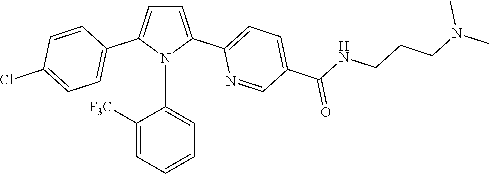 [SMILES: CN(C)CCCNC(=O)c1ccc(-c2ccc(-c3ccc(Cl)cc3)n2-c2ccccc2C(F)(F)F)nc1] | Compound B-62 |
| 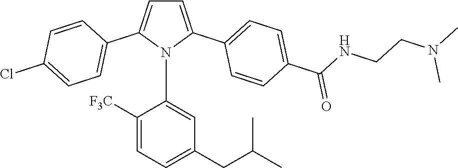 [SMILES: CC(C)Cc1ccc(C(F)(F)F)c(-n2c(-c3ccc(Cl)cc3)ccc2-c2ccc(C(=O)NCCN(C)C)cc2)c1] | Compound B-63 |
| 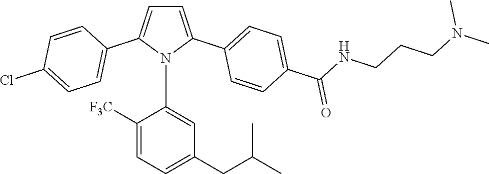 [SMILES: CC(C)Cc1ccc(C(F)(F)F)c(-n2c(-c3ccc(Cl)cc3)ccc2-c2ccc(C(=O)NCCCN(C)C)cc2)c1] | Compound B-64 |
| 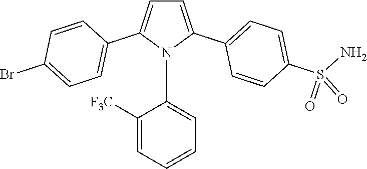 [SMILES: NS(=O)(=O)c1ccc(-c2ccc(-c3ccc(Br)cc3)n2-c2ccccc2C(F)(F)F)cc1] | Compound B-65 |
| 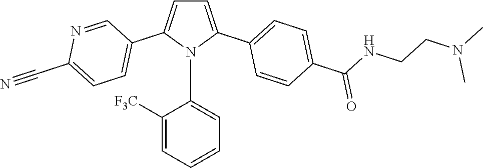 [SMILES: CN(C)CCNC(=O)c1ccc(-c2ccc(-c3ccc(C#N)nc3)n2-c2ccccc2C(F)(F)F)cc1] | Compound B-66 |
| 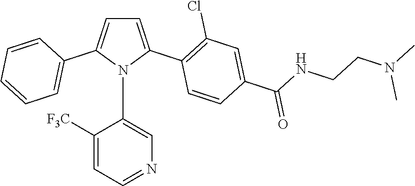 [SMILES: CN(C)CCNC(=O)c1ccc(-c2ccc(-c3ccccc3)n2-c2cnccc2C(F)(F)F)c(Cl)c1] | Compound B-67 |
| 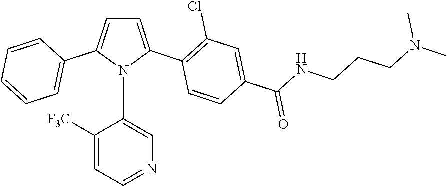 [SMILES: CN(C)CCCNC(=O)c1ccc(-c2ccc(-c3ccccc3)n2-c2cnccc2C(F)(F)F)c(Cl)c1] | Compound B-68 |
| 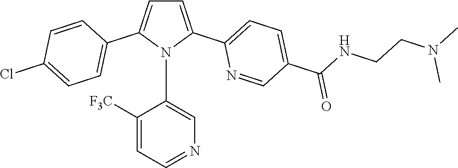 [SMILES: CN(C)CCNC(=O)c1ccc(-c2ccc(-c3ccc(Cl)cc3)n2-c2cnccc2C(F)(F)F)nc1] | Compound B-69 |
| 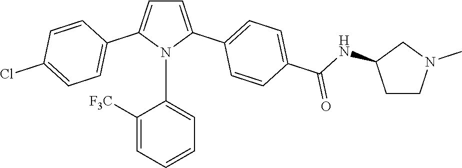 [SMILES: CN1CC[C@@H](NC(=O)c2ccc(-c3ccc(-c4ccc(Cl)cc4)n3-c3ccccc3C(F)(F)F)cc2)C1] | Compound B-70 |
| 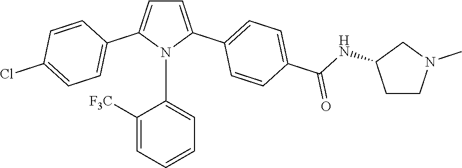 [SMILES: CN1CC[C@H](NC(=O)c2ccc(-c3ccc(-c4ccc(Cl)cc4)n3-c3ccccc3C(F)(F)F)cc2)C1] | Compound B-71 |
| 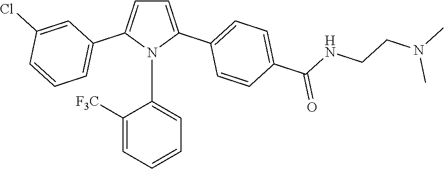 [SMILES: CN(C)CCNC(=O)c1ccc(-c2ccc(-c3cccc(Cl)c3)n2-c2ccccc2C(F)(F)F)cc1] | Compound B-72 |
| 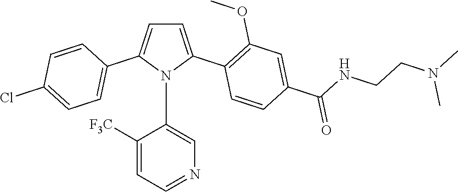 [SMILES: COc1cc(C(=O)NCCN(C)C)ccc1-c1ccc(-c2ccc(Cl)cc2)n1-c1cnccc1C(F)(F)F] | Compound B-73 |
| 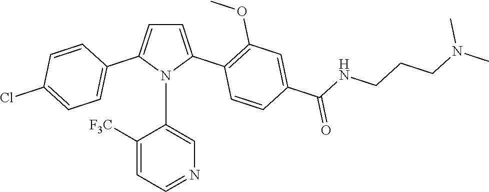 [SMILES: COc1cc(C(=O)NCCCN(C)C)ccc1-c1ccc(-c2ccc(Cl)cc2)n1-c1cnccc1C(F)(F)F] | Compound B-74 |
| 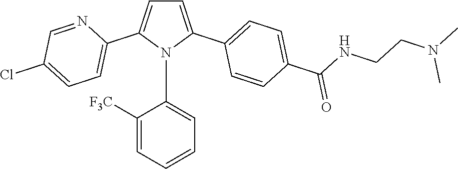 [SMILES: CN(C)CCNC(=O)c1ccc(-c2ccc(-c3ccc(Cl)cn3)n2-c2ccccc2C(F)(F)F)cc1] | Compound B-75 |
| 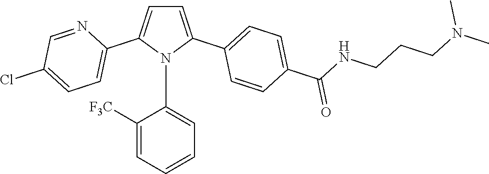 [SMILES: CN(C)CCCNC(=O)c1ccc(-c2ccc(-c3ccc(Cl)cn3)n2-c2ccccc2C(F)(F)F)cc1] | Compound B-76 |
| 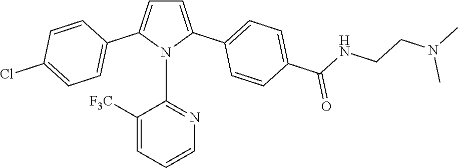 [SMILES: CN(C)CCNC(=O)c1ccc(-c2ccc(-c3ccc(Cl)cc3)n2-c2ncccc2C(F)(F)F)cc1] | Compound B-79 |
| 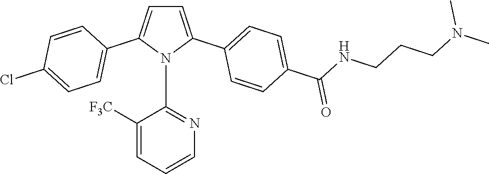 [SMILES: CN(C)CCCNC(=O)c1ccc(-c2ccc(-c3ccc(Cl)cc3)n2-c2ncccc2C(F)(F)F)cc1] | Compound B-80 |
| 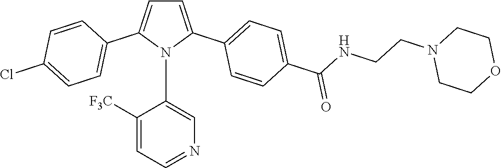 [SMILES: O=C(NCCN1CCOCC1)c1ccc(-c2ccc(-c3ccc(Cl)cc3)n2-c2cnccc2C(F)(F)F)cc1] | Compound B-81 |
| 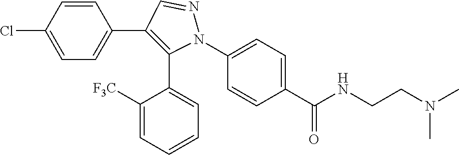 [SMILES: CN(C)CCNC(=O)c1ccc(-n2ncc(-c3ccc(Cl)cc3)c2-c2ccccc2C(F)(F)F)cc1] | Compound B-82 |
| 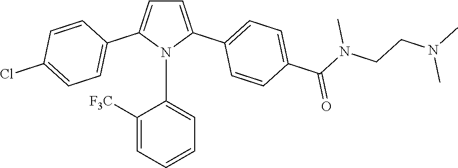 [SMILES: CN(C)CCN(C)C(=O)c1ccc(-c2ccc(-c3ccc(Cl)cc3)n2-c2ccccc2C(F)(F)F)cc1] | Compound B-83 |
| 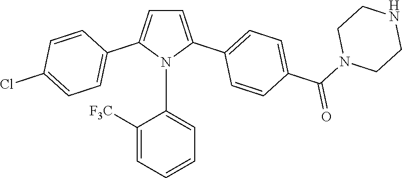 [SMILES: O=C(c1ccc(-c2ccc(-c3ccc(Cl)cc3)n2-c2ccccc2C(F)(F)F)cc1)N1CCNCC1] | Compound B-84 |
| 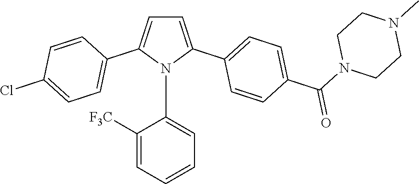 [SMILES: CN1CCN(C(=O)c2ccc(-c3ccc(-c4ccc(Cl)cc4)n3-c3ccccc3C(F)(F)F)cc2)CC1] | Compound B-85 |
| 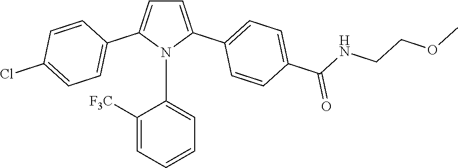 [SMILES: COCCNC(=O)c1ccc(-c2ccc(-c3ccc(Cl)cc3)n2-c2ccccc2C(F)(F)F)cc1] | Compound B-86 |
| 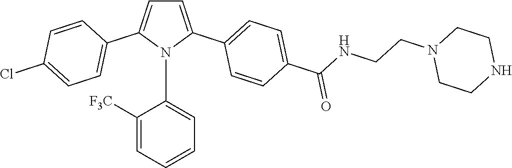 [SMILES: O=C(NCCN1CCNCC1)c1ccc(-c2ccc(-c3ccc(Cl)cc3)n2-c2ccccc2C(F)(F)F)cc1] | Compound B-87 |
| 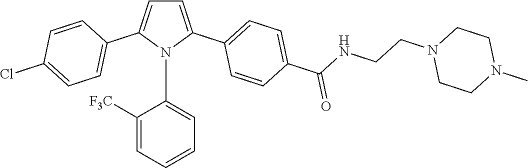 [SMILES: CN1CCN(CCNC(=O)c2ccc(-c3ccc(-c4ccc(Cl)cc4)n3-c3ccccc3C(F)(F)F)cc2)CC1] | Compound B-88 |
| 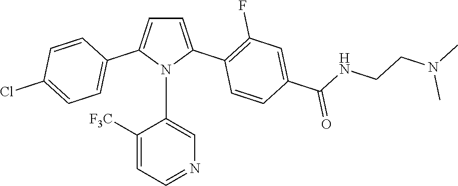 [SMILES: CN(C)CCNC(=O)c1ccc(-c2ccc(-c3ccc(Cl)cc3)n2-c2cnccc2C(F)(F)F)c(F)c1] | Compound B-89 |
| 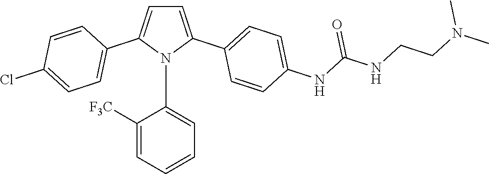 [SMILES: CN(C)CCNC(=O)Nc1ccc(-c2ccc(-c3ccc(Cl)cc3)n2-c2ccccc2C(F)(F)F)cc1] | Compound B-90 |
| 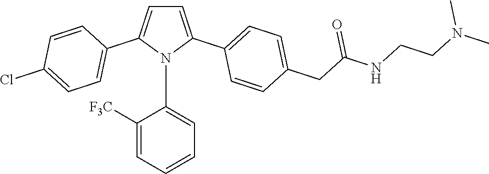 [SMILES: CN(C)CCNC(=O)Cc1ccc(-c2ccc(-c3ccc(Cl)cc3)n2-c2ccccc2C(F)(F)F)cc1] | Compound B-91 |
| 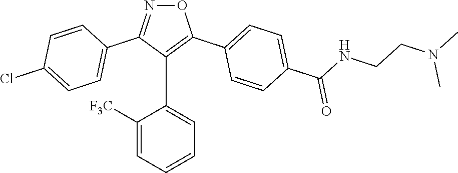 [SMILES: CN(C)CCNC(=O)c1ccc(-c2onc(-c3ccc(Cl)cc3)c2-c2ccccc2C(F)(F)F)cc1] | Compound B-92 |
| 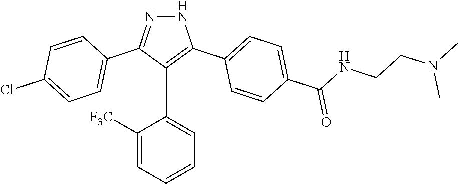 [SMILES: CN(C)CCNC(=O)c1ccc(-c2[nH]nc(-c3ccc(Cl)cc3)c2-c2ccccc2C(F)(F)F)cc1] | Compound B-93 |
| 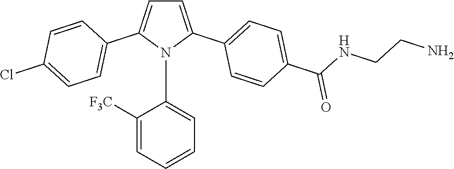 [SMILES: NCCNC(=O)c1ccc(-c2ccc(-c3ccc(Cl)cc3)n2-c2ccccc2C(F)(F)F)cc1] | Compound B-94 |
| 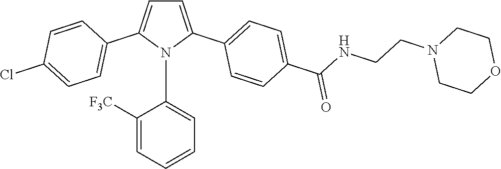 [SMILES: O=C(NCCN1CCOCC1)c1ccc(-c2ccc(-c3ccc(Cl)cc3)n2-c2ccccc2C(F)(F)F)cc1] | Compound B-95 |
| 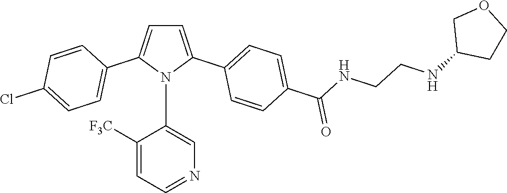 [SMILES: O=C(NCCN[C@H]1CCOC1)c1ccc(-c2ccc(-c3ccc(Cl)cc3)n2-c2cnccc2C(F)(F)F)cc1] | Compound B-96 |
| 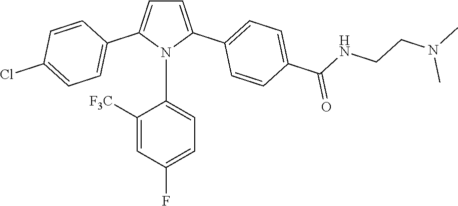 [SMILES: CN(C)CCNC(=O)c1ccc(-c2ccc(-c3ccc(Cl)cc3)n2-c2ccc(F)cc2C(F)(F)F)cc1] | Compound B-97 |
| 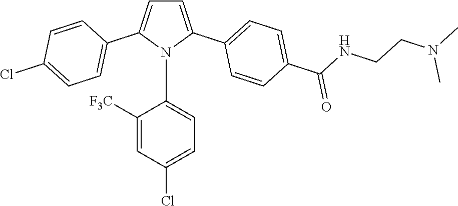 [SMILES: CN(C)CCNC(=O)c1ccc(-c2ccc(-c3ccc(Cl)cc3)n2-c2ccc(Cl)cc2C(F)(F)F)cc1] | Compound B-98 |
| 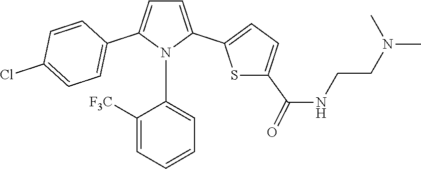 [SMILES: CN(C)CCNC(=O)c1ccc(-c2ccc(-c3ccc(Cl)cc3)n2-c2ccccc2C(F)(F)F)s1] | Compound B-99 |
| 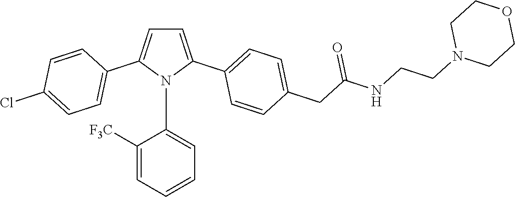 [SMILES: O=C(Cc1ccc(-c2ccc(-c3ccc(Cl)cc3)n2-c2ccccc2C(F)(F)F)cc1)NCCN1CCOCC1] | Compound B-100 |
| 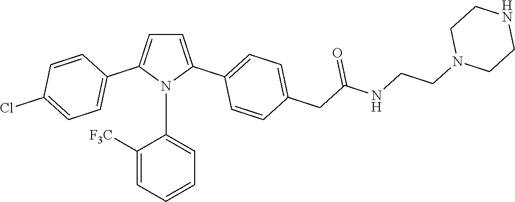 [SMILES: O=C(Cc1ccc(-c2ccc(-c3ccc(Cl)cc3)n2-c2ccccc2C(F)(F)F)cc1)NCCN1CCNCC1] | Compound B-101 |
| 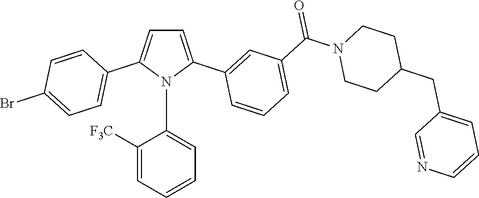 [SMILES: O=C(c1cccc(-c2ccc(-c3ccc(Br)cc3)n2-c2ccccc2C(F)(F)F)c1)N1CCC(Cc2cccnc2)CC1] | Compound B-102 |
| 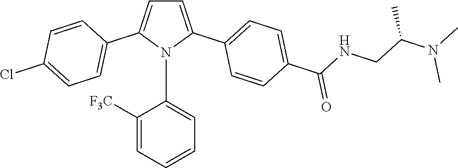 [SMILES: C[C@@H](CNC(=O)c1ccc(-c2ccc(-c3ccc(Cl)cc3)n2-c2ccccc2C(F)(F)F)cc1)N(C)C] | Compound B-103 |
| 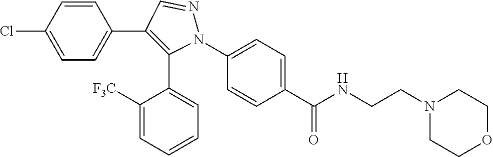 [SMILES: O=C(NCCN1CCOCC1)c1ccc(-n2ncc(-c3ccc(Cl)cc3)c2-c2ccccc2C(F)(F)F)cc1] | Compound B-104 |
| 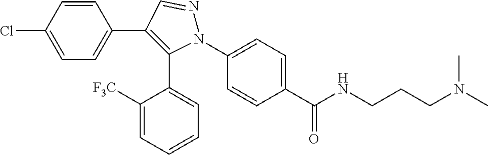 [SMILES: CN(C)CCCNC(=O)c1ccc(-n2ncc(-c3ccc(Cl)cc3)c2-c2ccccc2C(F)(F)F)cc1] | Compound B-105 |
| 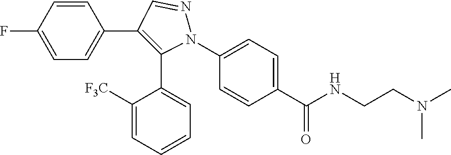 [SMILES: CN(C)CCNC(=O)c1ccc(-n2ncc(-c3ccc(F)cc3)c2-c2ccccc2C(F)(F)F)cc1] | Compound B-106 |
| 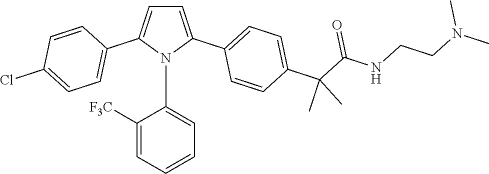 [SMILES: CN(C)CCNC(=O)C(C)(C)c1ccc(-c2ccc(-c3ccc(Cl)cc3)n2-c2ccccc2C(F)(F)F)cc1] | Compound B-107 |

### Example 8

### Alternative Method for Preparing (R)-4-[5-(4-chlorophenyl)-1-[2-(trifluoromethyl)phenyl]pyrrol-2-yl]-N-[2 (dimethylamino)ethyl]benzamide (Atropisomer A-2)

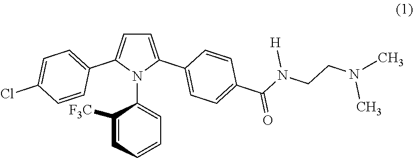

The title compound was prepared by following Steps 1, 2, 3, 4a and 5a of the synthetic routes shown in Scheme 1 above. In this route, chiral resolution is carried out on the carboxylic acid intermediate (8) rather than on the dimethylamino-ethyl amide (9).

### Step 1: 4-[4-(4-chlorophenyl)-4-oxo-butanoyl]benzonitrile (6)

A flask was charged with tetrahydrofuran (4 mL/g) and zinc chloride (1.222 g/g, 1.3 eq.) was added in portions to afford a white mobile suspension which was stirred for 15 min. tert-butanol (0.66 mL/g, 1 eq) was added followed by triethylamine (0.96 mL/g, 1 eq) in portions keeping the temperature below 40° C. The reaction was stirred for 2 h. 4-Cyanoacetophenone (1 g/g, 1 eq) and 4-chlorophenacyl bromide (1.61 g/g, 1 eq) were added and the reaction mixture was stirred at 20° C. (±5) for 48 h or until reaction was complete. The product was isolated by precipitation with aqueous HCl and slurry in aqueous HCl and methanol. The resulting solid was dried under vacuum (45° C.) to afford the title compound as a pale yellow solid.

### Step 2: 4-(5-(4-chlorophenyl-1-(2-(trifluoromethyl) phenyl)-1H-pyrrol-2-yl) benzonitrile (7)

4-(4-(4-chlorophenyl)-4-oxobutanoyl) benzonitrile (1 g/g, 1 eq) was charged to a flask and dioxane (10 mL/g) was added to afford a yellow suspension. 2-Trifluoromethyl aniline (1.269 mL/g, 3 eq) was added in a single portion followed by p-toluenesulfonic acid (0.06399 g/g, 0.1 eq) and the reaction mixture was heated at 101° C. for 40-72 h (additional portions of p-toluenesulfonic acid (0.1 eq) were added if required every 8 hours to push the reaction to completion). The reaction mixture was cooled to room temperature and concentrated under vacuum. The resulting oily residue was purified by slurring in methanol (10 mL/g). The solid was isolated by filtration and dried under vacuum (45° C.) to afford the title compound as a yellow solid.

### Step 3: 4-(5-(4-chlorophenyl)-1-(2-(trifluoromethyl) phenyl)-1H-pyrrol-2-yl) benzoic acid (8)

To 4-(5-(4-chlorophenyl)-1-(2-(trifluoromethyl) phenyl)-1H-pyrrol-2-yl) benzonitrile (1 g/g, 1 eq) in methanol (10.9 mL/g) was added sodium hydroxide (0.948 g/g, 10 eq) in water (5 mL/g) dropwise over 15 minutes and the resulting mixture was stirred at 70-76° C. for 18 hours or until complete. The reaction mixture was cooled to room temperature, acidified and the product isolated by filtration, washing with water (5 mL/g) and acetonitrile (3 mL/g). The product was slurried in acetone/water (20 vols, 75:25) at 50-55° C. and dried under vacuum (60° C.) to afford the title compound as a yellow solid.

### Step 4a: (R) 4-(5-(4-chlorophenyl)-1-(2-(trifluoromethyl) phenyl)-1H-pyrrol-2-yl) benzoic acid (3) by chiral resolution of (8)

4-(5-(4-chlorophenyl)-1-(2-(trifluoromethyl) phenyl)-1H-pyrrol-2-yl) benzoic acid (1 g/g, 1 eq) was added to a flask followed by tetrahydrofuran (2 mL/g) and acetonitrile (0.75 mL/g). (S)-1-(4-methoxyphenyl)-ethylamine (0.335 mL/g, 1 eq) was added dropwise over 5 min and the resulting reaction mixture was stirred at 40-50° C. for 15 min then cooled to room temperature. Acetonitrile (7.25 mL/g) was added and the reaction seeded (0.0001 g/g, 99% ee, (S)-1-(4-methoxyphenyl)-ethylamine salt of desired atropisomer). The reaction mixture was stirred for 16 h and the resulting solids were isolated by filtration washing with acetonitrile. Hot (75-80° C.) slurry in acetonitrile afforded the chiral salt as a white solid (40% yield, 98.16% ee). Salt break was achieved in THF/water (2/2 vols) using 1M HCl (2.2 eq) to afford the acid which was further purified by slurry in water affording the title compound (90.52 g, salt break yield 97%, overall yield 39%, 98.06% ee). ¹H NMR (DMSO-d6) δ 12.83 (brs, 1H), 7.77-7.67 (m, 6H), 7.23-7.10 (m, 2H), 7.08-7.01 (m, 4H), 6.68 (d, J=4.0 Hz, 1H), 6.59-6.58 (d, J=4.0 Hz, 1H). Chiral HPLC with chiral HPLC method 6 showed a single atropisomer, RT 6.083 min, 99.02% area (minor atropisomer RT 7.07 min, 0.98% area).

Chiral resolution can also be achieved using (S)-(−)-1-phenylethylamine.

### Step 5a: (R)-4-[5-(4-chlorophenyl)-1-[2-(trifluoromethyl)phenyl]pyrrol-2-yl]-N-[2 (dimethylamino)ethyl]benzamide (1)

4-(5-(4-chlorophenyl)-1-(2-(trifluoromethyl) phenyl)-1H-pyrrol-2-yl) benzoic acid (single atropisomer) (1 g/g, 1 eq) was dissolved in THF (5 mL/g) and N,N-dimethylethylenediamine (0.75 mL/g, 3 eq) was added dropwise followed by DIPEA (1.58 mL/g, 4 eq). 50% T3P in THF (2.72 mL/g, 2 eq) was added dropwise and the reaction mixture stirred at 20° C. for 15 min. Additional portions of 50% T3P in THF were added until reaction was complete. The reaction mixture was diluted with 10% brine (2 mL/g) and sodium hydroxide solution (2 mL/g) until pH8-10. The layers were separated, and the aqueous layer extracted with ethyl acetate (2×5 mL/g). The combined organic layers were washed with brine, dried (MgSO4) and concentrated to afford the title compound (80 g, 156 mmol, 71%) as a white triboluminescent solid. Chiral HPLC with chiral HPLC method 7 showed a single atropisomer, RT 12.62 min, 99.32% area (minor atropisomer, RT 10.58 min, 0.67% area)

### Example 9

### Preoaration and characterisation of (R)-4-[5-(4-chlorophenyl)-1-[2-(trifluoromethyl)phenyl]pyrrol-2-yl]-N-[2 (dimethylamino)ethyl]benzamide tartrate

Method 1: Small Scale Preparation of Tartrate Salt

Atropisomer A-2 free base (904.2 mg) was suspended in acetone (9.042 mL, 10 vols) and stirred at 25° C. for 40 minutes. When the solution was free of visible particulates, it was split into 12 equal aliquots (603 μL), giving an approximate active content of 60.3 mg per sample.

An aliquot of 247 μL (1.05 eq) of a 0.5 M solution of tartaric acid in ethanol was added to an aliquot of the free base solution at 25° C. The mixture was stirred at 25° C. for 18 hours after which time a white suspension formed, and the resulting solids were then isolated by filtration (PTFE 10 micron fritted cartridge) and the resulting solids were then isolated and dried in vacuo at 40° C. for ca. 72 hours.

The resulting salt was labelled as Tartrate Pattern A (solvate).

Method 2: Preparation of Tartrate Salt Using an Isopropyl Acetate Solution of Atropisomer A-2

Atropisomer A-2 (749.8 mg) was suspended in isopropyl acetate (15 mL, 20 vols) and the suspension was heated to 40° C. with agitation. When the solution was free of visible particulates, it was split into 12 equal aliquots (1 ml), giving an approximate active content of 50 mg per sample. An aliquot of 195.3 μL of a 1 M solution of atropisomer A-2 in ethanol was added to an aliquot of the free base solution at 40° C. The resulting mixture was cooled to 25° C. at a cooling rate of approximately 10° C./hour. A white suspension formed and the resulting solids were then isolated by filtration (PTFE 10 micron fritted cartridge) and dried in vacuo at 40° C. for ca. 18 hours. The resulting salt was labelled as Tartrate Pattern B.

Method 3: Preparation of Tartrate Salt Using an Isopropyl Alcohol Solution of Atropisomer A-2

By following Method 2, except that atropisomer A-2 (750.1 mg) was initially suspended in isopropyl alcohol (15 ml, 20 vols), Tartrate Pattern A salt was prepared.

Method 4: Preparation of Tartrate Salt Using a 2-Methyl-Tetrahydrofuran Solution of Atropisomer A-2

Method 1 was repeated, except that atropisomer A-2 (913.9 mg) was initially suspended in 2-methyl-tetrahydrofuran (15 ml, 20 vols), (9.139 mL, 10 vols) and stirred at 25° C. for ca. 40 minutes, and then a 250 μl (1.05 eq) aliquot of 1 M tartaric acid in ethanol was added to an aliquot of the A-2 free base solution, to give Tartrate Pattern A salt.

Method 5: 500 mg Scale Preparation of Atropisomer A-2 Tartrate Pattern B Salt

Atropisomer A-2 free base (521.5 mg) was weighed into a glass vial and charged with isopropyl acetate (20 vols, 10.430 ml). The mixture was heated to 40° C. and stirred for 15 minutes to give a clear solution. The solution was then charged with tartaric acid (1.05 eq, 162.5 mg) dissolved in 3 mL of tetrahydrofuran. The resulting mixture was seeded with atropisomer A-2.tartrate pattern B, which caused the salt to immediately precipitate at 40° C. forming a mobile suspension.

The mixture was cooled to 25° C. and stirred for 20 hours. The resulting solid was isolated by filtration and dried at 40° C. in vacuo to afford the atropisomer A-2 Tartrate Pattern B salt in 84% yield.

Method 6: Scaled-Up Preparation of Atropisomer A-2 Tartrate Pattern B Salt (Anhydrous Form)

Atropisomer A-2 free base (10.0497 g) was weighed into a Buchi flask and charged with isopropyl acetate (20 vols, 200 ml). The mixture was heated to 40° C. to afford a clear solution, free of particulates, and stirred for 30 minutes. The solution was charged with tartaric acid (3.1954 g, 1.08 eq.) dissolved in tetrahydrofuran (50 mL), the acid being was added in portions as follows: 15 mL at 40° C.; seeded with atropisomer A-2 tartrate pattern B salt and stirred for 30 minutes; 10 mL and stirred for 1 hour; 10 mL and stirred for 30 minutes; 15 mL and stirred for 30 minutes. The white suspension was then cooled to RT at a cooling rate of 10° C./h and stirred for 18 hours. The resulting solid was isolated by filtration in vacuo and washed with isopropyl acetate (2×2 vols) and dried in vacuo at 40° C. for 20 hours to afford the A-2 Tartrate Pattern B salt (anhydrous) in a yield of 97%; HPLC purity 99.74% (HPLC method 1), chiral purity 99.27% (Chiral HPLC method 7).

Method 7: Alternative Scaled-Up Preparation of Atropisomer A-2 Tartrate Pattern B Salt (Anhydrous Form) by Cooling Crystallisation from Butanol/Water 96:4

Atropisomer A-2 free base (36.79 g) was weighed into a flask and charged with butanol (282.57 ml, 7.68 vols). The mixture was heated to 80° C. (pale yellow, hazy solution) and stirred for 30 minutes before clarification into a Mya* vessel, pre-heated at 80° C. The solution was then charged with L-(+)-tartaric acid (1.023 eq, 11.0806 g) as a solution in water (11.77 mL, 0.32 vols of the initial API charge). The addition was made dropwise at 80° C. with clarification of the acid solution. The mixture was then cooled to 68° C. over a period of 30 minutes, seeded with 0.1% of ground atropisomer A-2 tartrate Pattern B salt seed crystals (32.6 mg) and held for 1 hour. The mixture was then cooled to 5° C. at a cooling rate of 5° C./hour and stirred at 5° C. for 6 hours before isolation of the solid. The solid was filtered in vacuo, washed twice with butanol and dried for 15 minutes on the filter and then at 40° C. for 20 hours to afford atropisomer A-2 Tartrate Pattern B salt (anhydrous) in a yield of 83%; HPLC purity 99.84% (HPLC method 1), chiral purity 99.66% (Chiral HPLC method 7).

*Note: In the foregoing equilibrations or crystallisaions that required temperature control and/or defined heating/cooling profiles, a Radley's Mya4 Reaction Station was used. The Radley's Mya4 Reaction Station is a 4-zone reaction station with magnetic and overhead stirring capabilities and a temperature range of −30 to 180° C. on 2 to 400 mL scale mixtures. The reaction conditions required were programmed via the Mya 4 Control Pad.

Characterisation of the Atropisomer A-2 Tartrate Salts

The identities of the salts as 1:1 (molar ratio of free base:tartaric acid) stoichiometric salts were confirmed from their ¹H NMR spectra which were collected using a JEOL ECX 400 MHz spectrometer equipped with an auto-sampler. The samples were dissolved in a suitable deuterated solvent for analysis. The data were acquired using Delta NMR Processing and Control Software version 4.3.

The tartrate salts were characterised using X-ray powder diffraction (XRPD), differential scanning calorimetry (DSC), thermogravimetric analysis (TGA), gravimetric solubility tests and gravimetric vapour sorption tests using the techniques described below.

X-Ray Powder Diffraction (XRPD)

X-Ray Powder Diffraction patterns were collected on a PANalytical diffractometer using Cu Kα radiation (45 kV, 40 mA), θ-θ goniometer, focusing mirror, divergence slit (½″), soller slits at both incident and divergent beam (4 mm) and a PiXcel detector. The software used for data collection was X'Pert Data Collector, version 2.2f and the data was presented using X'Pert Data Viewer, version 1.2d. XRPD patterns were acquired under ambient conditions via a transmission foil sample stage (polyimide—Kapton, 12.7 μm thickness film) under ambient conditions using a PANalytical X'Pert PRO. The data collection range was 2.994-35°2θ with a continuous scan speed of 0.202004° s-1.

Differential Scanning Calorimetry (DSC)

DSC data were collected on a PerkinElmer Pyris 6000 DSC equipped with a 45-position sample holder. The instrument was verified for energy and temperature calibration using certified indium. A predefined amount of the sample, 0.5-3.0 mg, was placed in a pin-holed aluminium pan and heated at 20° C.min—from 30 to 350° C. or varied as experimentation dictated. A purge of dry nitrogen at 20 ml min⁻¹ was maintained over the sample. The instrument control, data acquisition and analysis were performed with Pyris Software v11.1.1 revision H.

Thermo-Gravimetric Analysis (TGA)

TGA data were collected on a PerkinElmer Pyris 1 TGA equipped with a 20-position auto-sampler. The instrument was calibrated using a certified weight and certified Alumel and Perkalloy for temperature. A predefined amount of the sample, 1-5 mg, was loaded onto a pre-tared aluminium crucible and heated at 20° C.min⁻¹ from ambient temperature to 400° C. A nitrogen purge at 20 ml·min⁻¹ was maintained over the sample. Instrument control, data acquisition and analysis were performed with Pyris Software v11.1.1 revision H.

Gravimetric Solubility

The solubility in water of the salts was measured using a gravimetric solubility protocol.

1 ml of water was charged into crystallisation tubes. The solid was weighed into a tared glass vial, added in portions to the solutions and the vial weighed after each addition until a hazy solution was observed. The amount in mg was then calculated to give the solubility in mg/ml.

The results obtained from the characterisation studies are set out in Table 8 below.

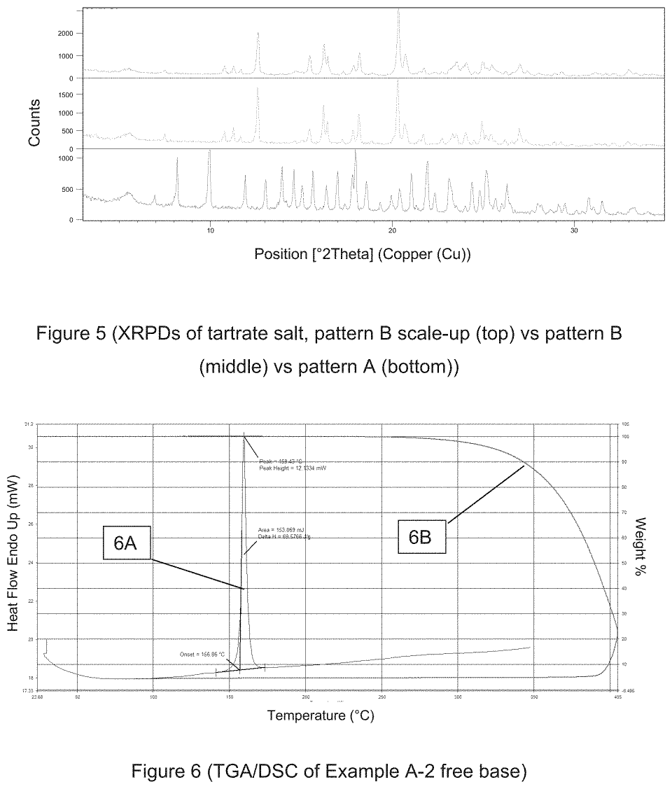

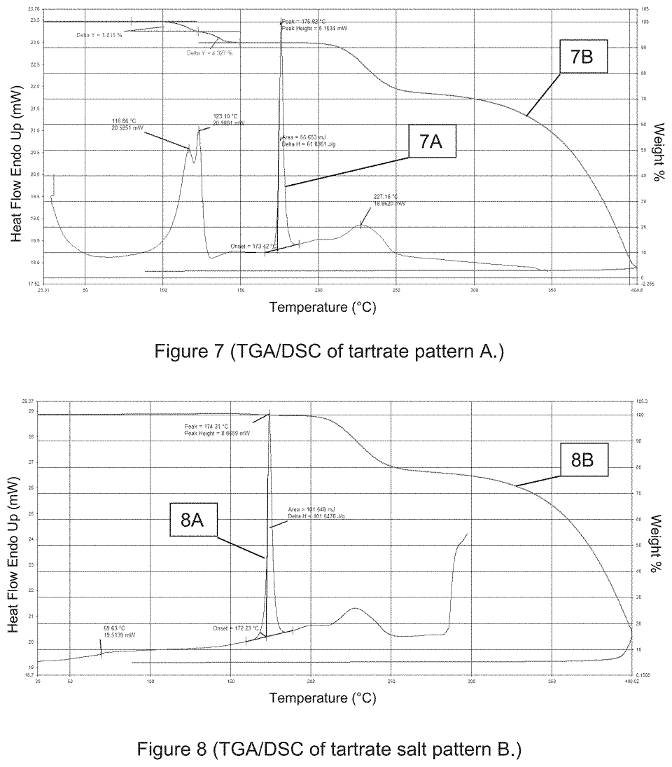

TABLE 8

| | | | | | Solubility |
|---|---|---|---|---|---|
| | XRPD | XRPD | | | in water |
| Salt | pattern | FIG. | DSC | TGA | (mg/mL) |
| Free | Pattern A | FIG. 4 | FIG. 6 line 6A | FIG. 6 | <5 |
| base | | | onset 157° C. | line 6B | |
| | | | peak 159° C. | | |
| Tartrate | Pattern A | FIG. 5 | FIG. 7 line 7A | FIG. 7 | 6.7 |
| | (EtOH | | onset~173° C. | line 7B | |
| | solvate) | | peak~175° C., | 78-157° C. | |
| | | | thermal event | loss 6% | |
| | | | 117° C. | | |
| Tartrate | Pattern B | FIG. 5 | FIG. 8 line 8A | FIG. 8 | 7.5 |
| | (anhydrous) | | onset~172° C. | line 8B | |
| | | | peak at~174° C. | | |

Gravimetric Vapour Sorption (GVS)

GVS studies were carried out on atropisomer A-2 Tartrate Pattern B salt using the protocol set out below:

Sorption isotherms were obtained using a Hiden Isochema moisture sorption analyser (model IGAsorp), controlled by IGAsorp Systems Software V6.50.48. The sample was maintained at a constant temperature (25° C.) by the instrument controls. The humidity was controlled by mixing streams of dry and wet nitrogen, with a total flow of 250 ml·min-1. The instrument was verified for relative humidity content by measuring three calibrated Rotronic salt solutions (10-50-88%). The weight change of the sample was monitored as a function of humidity by a microbalance (accuracy +/−0.005 mg). A defined amount of sample was placed in a tared mesh stainless steel basket under ambient conditions. A full experimental cycle typically consisted of three scans (sorption, desorption and sorption) at a constant temperature (25° C.) and 10% RH intervals over a 0-90% range (60 minutes for each humidity level). This type of experiment should demonstrate the ability of samples studied to absorb moisture (or not) over a set of well-determined humidity ranges

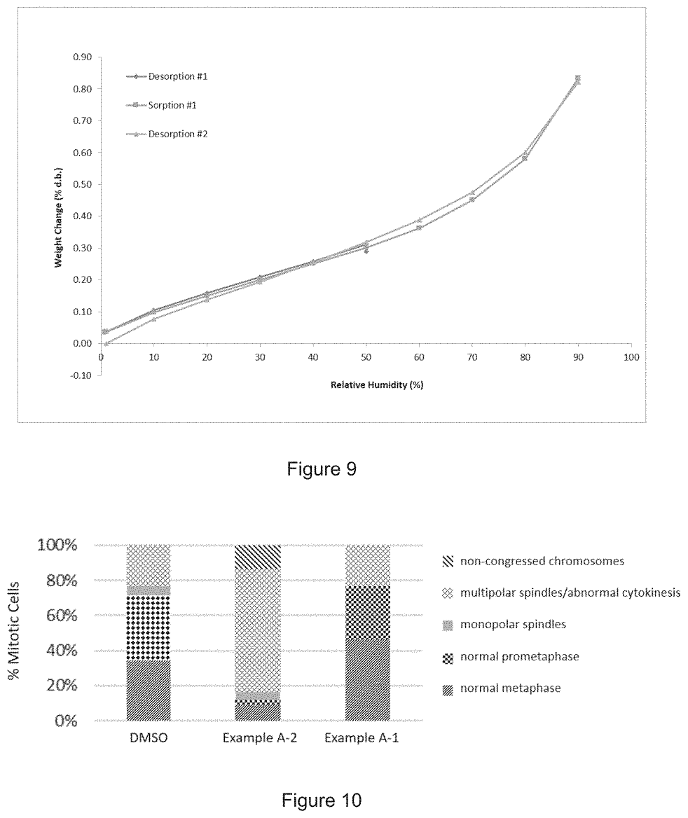

GVS analysis (see FIG. 9) indicated a moisture content of ca. 0.3% before the first desorption. Between 80 and 90% RH there is a slightly higher increase in moisture, with the solid taking ca. 0.8% moisture. The second absorption/desorption cycle shows how the moisture uptake is completely reversible, with a return to 0 wt % at 0% RH. XRPD post GVS cycling held at 0% RH and 90% RH for a minimum of 3 hours afforded anhydrous Pattern B at both RH values.

It can therefore be concluded that the atropisomer A-2 Tartrate Pattern B salt exists as a stable solid, only absorbing surface moisture with no change in form.

### Example 10

### Preoaration and characterisation of other salts of (R)-4-[5-(4-chlorophenyl)-1-[2-(trifluoromethyl)phenyl]pyrrol-2-yl]-N-[2 (dimethylamino)ethyl]benzamide

The hydrochloride, mesylate, maleate, malate, tosylate, sulfate and phosphate salts of (R)-4-[5-(4-chlorophenyl)-1-[2-(trifluoromethyl)phenyl]pyrrol-2-yl]-N-[2 (dimethylamino)ethyl]benzamide have been prepared and characterised. Their X-ray powder diffraction patterns (XRPD), thermal profiles (DSC and TGA) and solubilities in water are set out in the table below.

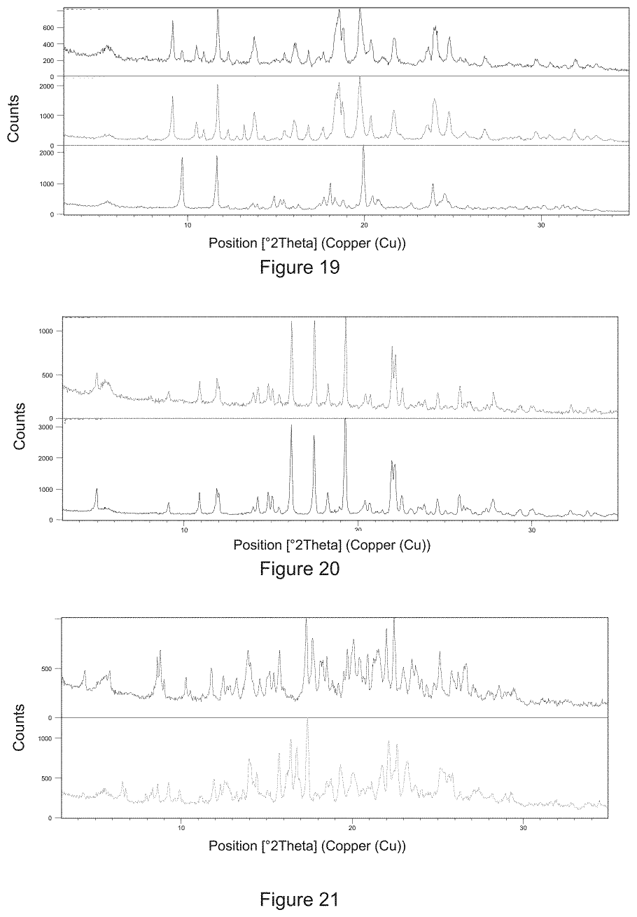

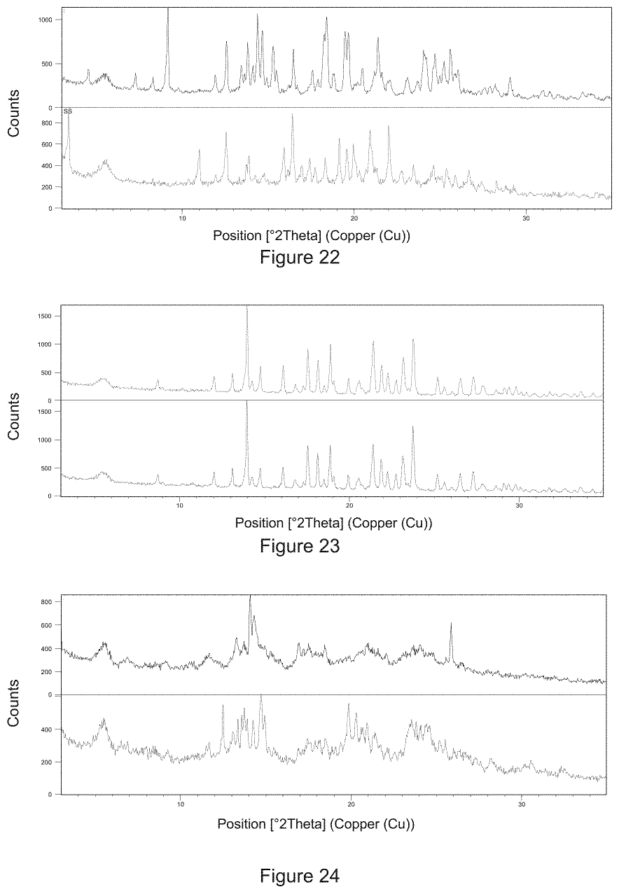

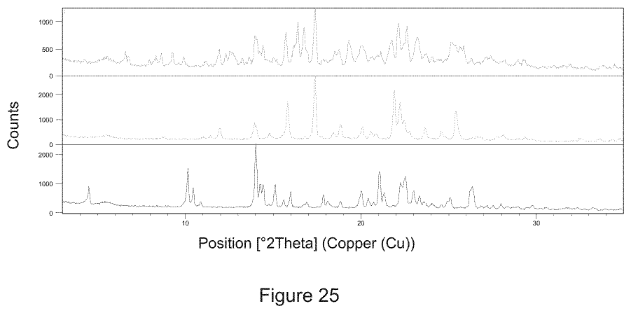

For all of the salts, ¹H NMR showed that there was a 1:1 ratio between free base and counterion.

| | | | Solubility |
|---|---|---|---|
| | XRPD | Thermal Profile | in water |
| Salt | FIG. | DSC/TGA | (mg/mL) |
| Hydrochloride | FIG. 19 | DSC events: | 12.9 |
| (XRPD Pattern A) | top 2 traces | onset~204° C. | |
| | | peak~206° C. | |
| | | TGA events: | |
| | | 30-207° C. loss 0.4% then | |
| | | decomposition | |
| Hydrochloride | FIG. 19 | DSC events: | n/a |
| (XRPD Pattern B) | bottom trace | onset~200° C. | |
| | | peak~205° C. | |
| Mesylate | FIG. 20 | DSC events: | 21.6 |
| (XRPD Pattern A) | | onset~162° C. | |
| | | peak~164° C. | |
| Maleate | FIG. 21 | DSC events: | n/a |
| (XRPD Pattern A | top trace | onset~115° C. | |
| (amorphous | | peak at~119° C., 2^{nd} | |
| content)) | | endotherm 132° C. | |
| | | thermal event~151° C. | |
| Maleate | FIG. 21 | DSC events: | 9.8 |
| (XRPD Pattern B- | bottom trace | onset~130° C. | |
| an hydrate) | | peak~132° C. | |
| Malate | FIG. 22 | DSC events: | n/a |
| (XRPD Pattern A) | bottom trace | onset 176° C. | |
| | | peak 211° C. | |
| | | glass transition~139° C., | |
| | | “shoulder” at 178° C. | |
| Malate | FIG. 22 | DSC events: | 2 |
| (XRPD Pattern B- | top trace | onset~184° C. | |
| solvate) | | peak~211° C., thermal | |
| | | events 64° C., 107° C. | |
| | | TGA events: | |
| | | 24-85° C. loss 1.5% | |
| | | 88-197° C. loss 6.1% | |
| Tosylate | FIG. 23 | DSC events: | <5 |
| (XRPD Pattern A) | | onset 176° C. | |
| | | peak 179° C. | |
| Phosphate | FIG. 24 | DSC events: | n/a |
| (XRPD Pattern A- | top trace | onset~162-164° C., | |
| anhydrous) | | peak~166° C.-167° C. | |
| | | thermal event 246° C. | |
| Phosphate | FIG. 24 | DSC events | n/a |
| (XRPD Pattern B) | bottom trace | onset~162-164° C. peak | |
| | | at~166-167° C., thermal | |
| | | events~137° C. and 225° C. | |
| Sulfate | FIG. 25 | DSC events: | n/a |
| (XRPD Pattern A) | top and | onset~176° C. | |
| | middle traces | peak at~183° C. | |
| | | decomposition after 300° C. | |
| Sulfate | FIG. 25 | DSC events: | n/a |
| (XRPD Pattern B) | bottom trace | onset~112° C. | |
| | | peak 118° C. | |
| | | melt/decomposition after | |
| | | 300° C. | |
| | | TGA events: | |
| | | 28-84° C. loss 0.9% | |
| | | 86-154° C. loss 2.7% | |

The solubility in water of the salts was measured using a gravimetric solubility protocol. Thus, 1 ml of water was charged into crystallisation tubes. The solid was weighed into a tared glass vial, added in portions to the solutions and the vial weighed after each addition until a hazy solution was observed. The amount in mg was then calculated to give the solubility in mg/mL.

Proton-NMR

¹H NMR spectra were collected using a JEOL ECX 400 MHz spectrometer equipped with an auto-sampler. The samples were dissolved in a suitable deuterated solvent for analysis. The data was acquired using Delta NMR Processing and Control Software version 4.3.

Preoaration of the Salts

Low Scale Preparation of Example A-2 Salts

Method 1: Acetone Mediated

Example A-2 free base (904.2 mg) was suspended in acetone (9.042 mL, 10 vols) and stirred at 25° C. for 40 minutes. When the solution was free of visible particulates, it was split into 12 equal aliquots (603 μL), giving an approximate active content of 60.3 mg per sample.

0.5 M or 1 M acid stock solutions (247 μL or 124 μL, 1.05 eq) in EtOH were charged to the solutions at 25° C. The mixtures were stirred at 25° C. for 18 hours. If required, the samples were manipulated further (e.g. by trituration of the solids and addition of anti-solvent) to recover solids for analysis, which were isolated and dried in vacuo at 40° C. for ca. 72 hours.

The amounts of acid used, the anti-solvent, and the resulting crystalline form are set out in the table below. Alternative methods can be used to isolate the salts.

| | Amount | | |
|---|---|---|---|
| | charged | | |
| Acid | (μl) | Anti-solvent | Crystalline Form |
| Hydrochloric | 124 | Et₂O | Hydrochloride Pattern A |
| Methane sulfonic | 124 | Heptane | Mesylate Pattern A |
| Maleic | 124 | Heptane | Maleate Pattern A |
| (−)-L-Malic | 124 | Gum isolated | Malate Pattern A |
| | | and dried to | |
| | | afford solid | |
| (+)-L-Tartaric | 247 | Not required | Tartrate Pattern A |
| | | | (solvate) |
| p-Toluenesulfonic | 124 | Not required | Tosylate Pattern A |
| Sulfuric | 124 | Et₂O | Sulfate Pattern A |

Method 2: Isopropyl acetate mediated

Example A-2 (749.8 mg) was suspended in iPrOAc (15 mL, 20 vols) heated to 40° C. with agitation. When the solution was free of visible particulates, it was split into 12 equal aliquots (1 ml), giving an approximate active content of 50 mg per sample. 0.5 M or 1 M acid stock solutions (195.3 μL or 97.7 μL, 1 eq) in EtOH were charged to the solutions at 40° C. The mixtures were cooled to 25° C. at approximately 10° C./h. If required, the samples were manipulated further (e.g. by trituration of the solids and addition of anti-solvent) to recover solids for analysis, which were isolated and dried in vacuo at 40° C. for ca. 18 h.

HCl pattern A (TBME anti-solvent), tartrate pattern B (1 M acid stock solution (195.3 μL) in EtOH), tosylate pattern A and phosphate pattern B can be isolated by Method 2

Method 3: IPA Mediated

Method identical to method 2 except that Example A-2 (750.1 mg) was suspended in IPA (15 mL, 20 vols).

HCl pattern A (TBME anti-solvent), tartrate pattern A (1 M acid stock solution (195.3 μL) in EtOH), tosylate pattern A and phosphate pattern A can be isolated by method 3.

Method 4: 2-Methyl THF Mediated

Method identical to method 1 except that Example A-2 (913.9 mg) was suspended in 2-Methyl THF (9.139 mL, 10 vols) and stirred at 25° C. for ca. 40 min and 0.5 M or 1 M acid stock solutions (250 μl or 125 μl, 1.05 eq) in EtOH were used.

HCl pattern B (heptane as anti-solvent), maleate pattern A (heptane as anti-solvent), tartrate pattern A (1 M acid (250 μl, 1.05 eq) in EtOH) and tosylate pattern A can be isolated by method 4.

A sub-set of salts were scaled up and more fully characterised.

500 Ma Scale Preparation of Example A-2 Salts

Hydrochloride Salt

Example A-2 free base (524.9 mg) was weighed into a glass vial and charged with IPA (20 vols, 10.498 ml) and heated to 40° C. The solution was stirred at 40° C. for 40 min and then charged with HCl (4.4 M in IPA, 1.2 eq, 280 μl). The mixture was then seeded with HCl salt pattern B and stirred at 40° C. for 15 min before being cooled down to 25° C. The mixture was concentrated in vacuo to afford a pale-yellow oil residue. The oil was suspended in 10 vols of TBME and stirred at 25° C. for 72 h, obtaining a white suspension. The solid was isolated and dried at 40° C. in vacuo for 18 h to afford the title salt pattern A in 73% yield.

Mesylate Salt

Example A-2 free base (503.9 mg) was weighed into a glass vial and charged with 2-Me THF (10 vols, 5.039 ml). The mixture was stirred at RT for 30 min. The solution was then charged with Methanesulfonic acid (1 M solution in EtOH, 1.05 eq, 1.033 ml), seeded with Example A-2.MsOH pattern A and stirred at 25° C. for 30 min. The mixture became a hazy solution and then formed a white suspension which was stirred at 25° C. for 72 h. The solid was isolated by filtration and dried in vacuo at 40° C. for 18 h to afford the title salt pattern A in 46% yield.

Tartrate Salt

Example A-2 free base (521.5 mg) was weighed into a glass vial and charged with iPrOAc (20 vols, 10.430 ml). The mixture was heated to 40° C. and stirred for 15 min to deliver a clear solution. The solution was then charged with Tartaric acid (1.05 eq, 162.5 mg) dissolved in 3 mL of THF. The mixture was then seeded with Example A-2.tartrate pattern B, which caused the salt to immediately precipitate at 40° C. forming a mobile suspension. The mixture was cooled to 25° C. and stirred for 20 h. The solid was isolated by filtration and dried at 40° C. in vacuo to afford the title salt pattern B in 84% yield.

Tosylate Salt

Example A-2 free base (504.5 mg), was weighed into a glass vial charged with iPrOAc (20 vols, 10.090 ml) and heated to 40° C. The solution was stirred at 40° C. for 40 min and then charged with p-toluenesulfonic acid (1 M in EtOH, 1.05 eq, 1.04 ml). The mixture was then seeded with a small amount of Example A-2.tosylate pattern A and stirred at 40° C. for 15 min before being cooled to 25° C. The mixture quickly became a white suspension and it was stirred at 25° C. for 72 h. The solid was isolated and dried at 40° C. in vacuo for 18 h to afford the title salt pattern A in 82% yield.

Maleate Salt

Example A-2 free base (523.9 mg) was weighed into a glass vial and charged with 2-Me THF (10 vols, 5.239 mL). The mixture was stirred at RT for 30 min, to give a clear solution. To the solution was then added Maleic acid (0.5 M in THF, 1.05 eq, 2.149 mL), seeded with a small amount of Example A-2.maleate pattern A and stirred at 25° C. for 30 min. The mixture was reduced in vacuo to yield a white gum. The gum was suspended in 10 vols of heptane and stirred at 25° C. for 72 h. The solid was isolated and dried in vacuo at 40° C. for 18 h to afford the title salt pattern B. 1H NMR conforms to structure but indicates ˜1:0.8 stoichiometry.

Malate Salt

Example A-2 free base (524.9 mg) was weighed into a glass vial, charged with IPA (20 vols, 10.618 ml) and heated to 40° C. The solution was stirred at 40° C. for 40 min and then charged with Malic acid (1 M solution in EtOH, 1.05 eq, 1.09 ml). The mixture was then stirred at 40° C. for 15 min before being cooled down to 25° C. The mixture, which remained as a solution at 25° C., was reduced in vacuo leaving an oil residue. The oil was suspended in 10 vols of heptane and stirred at 25° C. for 70 h obtaining a white suspension. The solid was isolated and dried at 40° C. in vacuo for 18 h to afford the title salt pattern B.

Sulfate Salt

Example A-2 free base (520 mg) was weighed into a glass vial charged with acetone (10 vols, 5.2 mL). The mixture was stirred at RT for 30 min, to yield a clear solution.

The solution was charged with Sulphuric acid (1 M in EtOH, 1.05 eq, 1.066 ml), seeded with Example A-2.Sulfate pattern A and stirred at 25° C. for 30 min. The mixture remained as a solution, so it was reduced in vacuo with a gentle stream of Nitrogen, which left a white gum.

The gum was suspended in 10 vols of diethyl ether and stirred at 25° C. for 70 h. The solid was then isolated and dried in vacuo at 40° C. for 18 h to afford the title salt pattern A sim (similar but not identical to previously isolated sulfate salt pattern A).

### Example 11

Biological Activity

A. Assay to Measure the Effects of Compounds of the Invention on U87MG Human Glioblastoma Cancer Cell Viability

The following protocol was used to measure the effects of compounds of the invention on U87MG cell viability.

U87MG cells were grown in their recommended growth media/supplements (ATCC). Cells were seeded at a concentration of 5000 cells per well into 96 well plates overnight at 37° C., 5% CO₂. Cells were treated with relevant concentrations of test compound for 72 hours. After 72 hours incubation, viability was established using sulforhodamine B (SRB) colorimetric assay. Percentage viability was calculated against the mean of the DMSO treated control samples, and IC₅₀ values for inhibition of cell growth were calculated using GraphPad Prism software by nonlinear regression (4 parameter logistic equation).

From the results obtained by following the above protocol, the IC₅₀ values against the U87MG cell line of the atropisomers of the Examples were determined as shown in Table 9 below.

TABLE 9

| | IC₅₀ |
|---|---|
| Atropisomer | (μM) |
| A-1 | 4.6 |
| A-2 | 0.22 |
| A-3 | 0.15 |
| A-4 | 3.94 |
| A-5/A-6 | 0.73* |
| A-7/A-8 | 1.48* |

*Although separate atropisomers A-5 and A-6, and A-7 and A-8, were identified by chiral chromatography, the racemic mixtures were tested in the U87MG cell viability assay.

B. Assay to Measure the Effects of Atropisomers A-1 and A-2 on Cancer Cell Viability of a Diverse Cancer Cell Line Panel

Screening against diverse cancer cell lines was performed to identify tumour types displaying sensitivity to atropisomers A-1 and A-2. A panel of 48 cancer-derived cell lines was screened in a high-throughput proliferation assay using dilutions of atropisomers A-1/A-2. Cell lines that were screened included those representing cancer of the pancreas, large intestine/colorectum, lung, brain and nerves, and lymphoma and leukaemia cell lines. Cell lines were treated with serial half-log dilutions of compound and assayed 72 hours later for proliferation using CellTiter-Glo Assay (Promega). IC₅₀ values were calculated by fitting the dose-response data using a nonlinear regression model. The IC₅₀ values in micromolar for atropisomers A-1 and A-2 are shown in Table 10 below.

TABLE 10

| Cell Line | Tissue Origin | A-1 | A-2 |
|---|---|---|---|
| MIA PaCa-2 | Pancreas | 9.17 | 0.33 |
| PANC-1 | Pancreas | >10 | 0.83 |
| AsPC-1 | Pancreas | >10 | 2.9 |
| Capan-1 | Pancreas | >10 | 1.23 |
| Panc 10.05 | Pancreas | 9 | 1.14 |
| BxPC-3 | Pancreas | 6.97 | 0.3 |
| HOT 116 | Large intestine/Colorectum | 7.36 | 0.23 |
| LoVo | Large intestine/Colorectum | 8.36 | 0.54 |
| SVV620 | Large intestine/Colorectum | 8.35 | 0.19 |
| SW480 | Large intestine/Colorectum | 7.55 | 0.87 |
| COLO 205 | Large intestine/Colorectum | 6.75 | 0.38 |
| HT-29 | Large intestine/Colorectum | 4.53 | 0.45 |
| RKO | Large intestine/Colorectum | 6.17 | 0.16 |
| A549 | Lung | 9.19 | 0.3 |
| NCI-H460 | Lung | 8.13 | 0.3 |
| HCC44 | Lung | 8.26 | 0.48 |
| NCI-H1373 | Lung | >10 | 3.21 |
| NCI-H1792 | Lung | 7.73 | 0.25 |
| NCI-H1299 | Lung | 8.25 | 0.37 |
| NCI-H1975 | Lung | 5.14 | 0.63 |
| SK-MES-1 | Lung | 7.78 | 0.36 |
| U118 MG | Brain & Nerves | 7.6 | 0.47 |
| A-172 | Brain & Nerves | 7.07 | 0.34 |
| LN-229 | Brain & Nerves | 7.37 | 0.31 |
| SW1088 | Brain & Nerves | 8.49 | 0.77 |
| T98G | Brain & Nerves | 5.26 | 0.28 |
| D283 Med | Brain & Nerves | 9.26 | 0.31 |
| Daoy | Brain & Nerves | 7 | 0.18 |
| DOHH-2 | Blood/Lymphoma | 5.64 | 0.23 |
| HBL-1 | Blood/Lymphoma | 9.94 | 0.66 |
| OCI-LY-19 | Blood/Lymphoma | 6.67 | 0.22 |
| SU-DHL-6 | Blood/Lymphoma | 3.98 | 0.26 |
| U-2932 | Blood/Lymphoma | 4.47 | 0.28 |
| WSU-DLCL2 | Blood/Lymphoma | 6.74 | 0.36 |
| SU-DHL-2 | Blood/Lymphoma | 7.01 | 0.2 |
| Toledo | Blood/Lymphoma | >10 | 0.88 |
| JeKo-1 | Blood/Lymphoma | 7.94 | 0.19 |
| Z-138 | Blood/Lymphoma | 7.04 | 0.21 |
| GRANTA-519 | Blood/Lymphoma | 7.42 | 0.22 |
| JVM-2 | Blood/Lymphoma | 4.71 | 0.38 |
| Daudi | Blood/Lymphoma | 7.17 | 0.39 |
| NAMALWA | Blood/Lymphoma | 7.05 | 0.25 |
| Raji | Blood/Lymphoma | 3.91 | 0.36 |
| Ramos | Blood/Lymphoma | 3.99 | 0.34 |
| ML-2 | Blood/Leukaemia | 4.01 | 0.18 |
| KG-1 | Blood/Leukaemia | >10 | 0.46 |
| MV-4-11 | Blood/Leukaemia | 6.49 | 0.28 |
| Kasumi-1 | Blood/Leukaemia | 5.13 | 0.33 |

As can be seen from the data, atropisomer A-2 was a significantly more active cell growth inhibitor than atropisomer A-1 against all of the cell lines

C. Assay to Measure the Effects of Compounds of the Invention on Cells in Mitosis

Inhibiting the ability of PLK1 and PLK4 to bind to their partners through their PBDs is known to cause cells to arrest in mitosis. Experimentally, this can be measured by assessing the number of cells which are in mitosis at a certain time after treatment with a test compound by immunofluorescent detection of phosphorylated Histone H3 (pH3), a mark which is only present in mitotic cells. PLK1/4-PBD inhibitors are expected to cause a dose-dependent increase in pH3-positive cells, which is reported as Mitotic Index (MI)—the percentage of cells which, at a given time, are positive for this mitotic mark.

Distinct mitotic phenotypes are induced following inhibition of PLK1 and PLK4 in cells. Disruption of the PBD domain of PLK1 has been demonstrated to trigger mitotic arrest with non-congressed chromosomes, a distinct phenotype from the monopolar spindle phenotype induced by ATP-competitive PLK1 inhibitors (Hanisch et al., 2006 Mol. Biol. Cell 17, 448-459). Centriole assembly is controlled by PLK4, with inhibitors inducing a multipolar spindle phenotype due to centrosome defects which results in abnormal cyokinesis (Wong et al., 2015. Science 348(6239); 1155-1160).

The following protocol was used to measure the effects of atropisomer A-2 and atropisomer A-3 on arresting cells in mitosis.

Cells were plated at 10 000/well in 96-well plates and incubated overnight. The following day atropisomer A-2 stocks in DMSO were diluted in medium then added to cells with a maximum final DMSO concentration on cells of 0.2%. Cells were incubated with the compound for 24 hours then fixed in 3.7% formaldehyde. Cells were permeabilised with 0.1% Triton X-100 then incubated with anti-phospho-histone H3 (Ser10) antibody (Abcam). The cells were washed with PBS then incubated with AlexaFluor 488 labelled goat anti-rabbit IgG (Invitrogen) in the presence of 4 ug/mL Hoechst 33342 (Invitrogen). Cells were washed in PBS then imaged on an Arrayscan VTi HCS instrument using the Target Activation V4 Bioapplication. A user-defined threshold was applied to identify mitotic cells based on the intensity of phospho-histone H3 staining.

GraphPad Prism was used to plot % mitotic cells against compound concentration using log(inhibitor) vs response variable slope with least squares fitting and no constraints.

From the results obtained by following the above protocol, the EC₅₀ values and the percentages of cells in mitosis against the HeLa and U87MG cell lines were obtained for atropisomer A-2 and atropisomer A-3. The EC₅₀ values are shown in Table 11 below.

TABLE 11

| | U87MG | HeLa |
|---|---|---|
| | EC₅₀ | EC₅₀ |
| Example | (μM) | (μM) |
| A-2 | 0.09 | 0.03 |
| A-3 | 0.10 | 0.03 |

Phenotype Study

In a separate study, following the above protocol and using single compound concentrations of 0.03 μM for each of atropisomer A-1 and atropisomer A-2, the frequency of observed mitotic phenotypes in U87MG cells was manually assessed and classified into the following phenotypes: non-congressed chromosomes, multipolar spindles/abnormal cytokinesis, monopolar spindles, normal prometaphase, normal metaphase for each of A-1 and A-2. The results are shown in FIG. 10.

Results

The results presented in FIG. 10 demonstrate that the atropisomer A-2 has a much greater effect on disrupting normal mitosis than atropisomer A-1. Thus, with A-1, 76% of the cells displayed a normal mitotic phenotype, comparable to 77% of the cells treated with DMSO control, and 24% displayed abnormal cytokinesis compared to 23% treated with DMSO control. No evidence of a non-congressed chromosome phenotype was seen in either DMSO control or atropisomer A-1 treated cells. By contrast, treatment of the cells with the more active atropisomer A-2 resulted in only 17% of cells with normal mitotic phenotype, 70% with abnormal cytokinesis and 13% with non-congressed chromosomes. These phenotypes are consistent with disrupting PLK1 and PLK4 activity during mitosis.

D. Assay to Measure the Effects of Atropisomer A-2 on Centrosomes

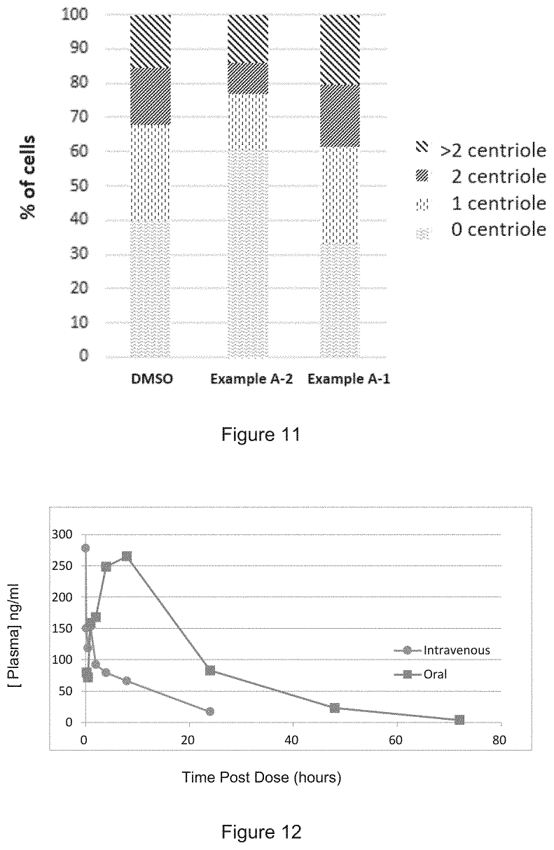

The results of study C above show that atropisomer A-2 causes mitotic effects which are characteristic of dysregulated centrosome function. The effects of A-1 and A-2 on centrosome function were therefore investigated further. HeLa cells stably expressing a Centrin1-GFP fusion protein were seeded into 96-well plates overnight. Cells were treated with atropisomer A-1 or atropisomer A-2 (at concentrations of 0.02 μM in DMSO) or DMSO control for 72 hours and then imaged using a fluorescence microscope. Multiple cell fields were captured for each treatment condition, and the images were subsequently analysed manually. Centrin1-GFP specifically marks centrioles as discrete foci, and therefore can be used to quantitate centriole number per cell. Thus, for each treatment condition, 100 cells were analysed and the number of centrioles present in each cell was recorded. The data were then separated into bins (no centrioles, 1 centriole, 2 centrioles, and greater than 2 centrioles) and are shown in FIG. 11.

From the data, it can be concluded that atropisomer A-2 exhibits evidence of PLK4 inhibition phenotypes on HeLa cells.

E. Assay to Measure the Effects of Compounds of the Invention on Wild-Type Versus KRAS HeLa Cell Viability

Atropisomers A-1, A-2, A-3 and A-4 were tested on HeLa cells engineered to inducibly express wild-type or oncogenic KRasG12V transgenes using the FLP-in/T-Rex system (Invitrogen). Cells were plated, and then treated with or without Doxycycline to induce transgene expression, and then treated with serially-diluted PBD inhibitors. After 72 hours of incubation, cell viability was assessed using the Cell Titre Blue reagent (Promega) and a BMG Pherastar plate reader. The effect of PBD inhibition on cell viability with either wild-type or oncogenic G12V KRAS was assessed using GraphPad Prism.

From the results obtained by following the above protocol, the GI₅₀ values against the wild-type and KRAS G12V HeLa cell line of each of the atropisomers were determined as shown in Table 12.

TABLE 12

| | WT | G12V |
|---|---|---|
| | GI₅₀ | GI₅₀ |
| Atropisomer | (nM) | (nM) |
| A-1 | NA | NA |
| A-2 | 3.01 | 2.08 |
| A-3 | 4.68 | 2.6 |
| A-4 | 254 | 153 |

F. Kinase Selectivity Assay

Compounds of the invention bind to the PBD domain of PLK1 and PLK4 but not to the catalytic domains of PLK1 and PLK4 and should exhibit good selectivity over other kinases. Atropisomer A-2 has been tested for off-target activity against a panel of ninety-seven kinases distributed across the kinome at a concentration of 3 μM using the DiscoverX KinomeScreen assay. The results are shown in Table 13 below.

The DiscoverX KinomeScreen assay is a site-directed competition binding assay which measures the binding affinity of a compound to a kinase, by use of a solid supported control compound which can bind or capture the kinases in solution. In the absence of a kinase-inhibitor test compound, all of the kinase will bind to the solid support. If a kinase-inhibitor test compound is added to the assay mix, the amount of kinase binding to the solid support will be reduced, the extent of reduction being dependent on the potency of the test compound as a kinase inhibitor. The potencies of the test compounds against the kinases can be expressed as the percentage (Percent Control) of the kinase binding to the solid support at a given concentration of the test compound, the lower the percentage the more potent the kinase-binding capability of the test compound. Thus, a Percent Control value of 100% would indicate that the test compound does not bind to the kinase at all, since all of the kinase has bound to the solid support. Conversely, a Percent Control value of 0% would indicate that the test compound has bound all of the kinase since none is bound to the solid support.

Protocol:

For most assays, kinase-tagged T7 phage strains were grown in parallel in 24-well blocks in an E. coli host derived from the BL21 strain.

E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 40× stocks in 100% DMSO and directly diluted into the assay. All reactions were performed in polypropylene 384-well plates in a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

Compounds that bind the kinase active site and directly (sterically) or indirectly (allosterically) prevent kinase binding to the immobilized ligand, will reduce the amount of kinase captured on the solid support. Conversely, test molecules that do not bind the kinase have no effect on the amount of kinase captured on the solid support.

The strength of binding of the test molecule to the kinase can be expressed as the percent control (% Ctrl)

Percent Control (% Ctrl)

The compound(s) were screened at 3000 nM concentration, and results for primary screen binding interactions are reported as ‘% Ctrl’, where lower numbers indicate stronger hits in the matrix on the following page(s).

% Ctrl Calculation

$$( \frac{test compound signal - positive control signal}{negative control signal - positive control signal} ) \times 100$$

negative control=DMSO (100% Ctrl)
positive control=control compound (0% Ctrl)

The % Ctrl values for atropisomer A-2 against the panel kinases are set out in Table 13 below.

TABLE 13

| Gene | % Ctrl @ |
|---|---|
| Symbol | 3 μM |
| ABL1 | 100 |
| ABL1 | 100 |
| ABL1 | 100 |
| ABL1 | 97 |
| ACVR1B | 96 |
| CABC1 | 63 |
| AKT1 | 98 |
| AKT2 | 100 |
| ALK | 89 |
| AURKA | 98 |
| AURKB | 84 |
| AXL | 93 |
| BMPR2 | 82 |
| BRAF | 96 |
| BRAF | 100 |
| BTK | 94 |
| CDK19 | 81 |
| CDK2 | 94 |
| CDK3 | 100 |
| CDK7 | 100 |
| CDK9 | 97 |
| CHEK1 | 100 |
| CSF1R | 100 |
| CSNK1D | 91 |
| CSNK1G2 | 100 |
| DCLK1 | 87 |
| DYRK1B | 97 |
| EGFR | 100 |
| EGFR | 100 |
| EPHA2 | 100 |
| ERBB2 | 66 |
| ERBB4 | 99 |
| MAPK3 | 99 |
| PTK2 | 98 |
| FGFR2 | 85 |
| FGFR3 | 92 |
| FLT3 | 100 |
| GSK3B | 88 |
| IGF1R | 100 |
| CHUK | 100 |
| IKBKB | 96 |
| INSR | 100 |
| JAK2 | 80 |
| JAK3 | 100 |
| MAPK8 | 77 |
| MAPK9 | 80 |
| MAPK10 | 93 |
| KIT | 100 |
| KIT | 100 |
| KIT | 100 |
| STK11 | 81 |
| MAP3K4 | 93 |
| MAPKAPK2 | 75 |
| MARK3 | 100 |
| MAP2K1 | 84 |
| MAP2K2 | 81 |
| MET | 100 |
| MKNK1 | 92 |
| MKNK2 | 85 |
| MAP3K9 | 96 |
| MAPK14 | 100 |
| MAPK11 | 99 |
| PAK1 | 87 |
| PAK2 | 100 |
| PAK4 | 100 |
| CDK16 | 100 |
| PDGFRA | 100 |
| PDGFRB | 100 |
| PDPK1 | 96 |
| PIK3C2B | 100 |
| PIK3CA | 100 |
| PIK3CG | 100 |
| PIM1 | 99 |
| PIM2 | 100 |
| PIM3 | 98 |
| PRKACA | 100 |
| PLK1 | 97 |
| PLK3 | 87 |
| PLK4 | 100 |
| PRKCE | 99 |
| RAF1 | 92 |
| RET | 95 |
| RIOK2 | 100 |
| ROCK2 | 88 |
| RPS6KA3 | 100 |
| NUAK2 | 99 |
| SRC | 100 |
| SRPK3 | 71 |
| TGFBR1 | 95 |
| TEK | 99 |
| NTRK1 | 100 |
| TSSK1B | 100 |
| TYK2 | 100 |
| ULK2 | 93 |
| KDR | 100 |
| STK32C | 100 |
| ZAP70 | 92 |

The results against ninety-seven kinases demonstrate that atropisomer A-2 has poor or non-existent binding activity against a wide range of kinases and therefore is unlikely to suffer from problems associated with off-target kinase inhibition.

In the case of PLK1 and PLK4, atropisomer A-2 showed little or no binding affinity for the catalytic domains of these kinases (% Control values of 97% and 100% respectively). It is concluded therefore that the activity profiles indicative of PLK1/PLK4 inhibitory activity demonstrated in the examples above is a consequence of to the non-catalytic polo box domains of PLK1 and PLK4.

G. Determination of Oral Bioavailability and Brain Exposure in Mouse PK

Atropisomers A-2 and A-3 were evaluated in an in vivo mouse model to determine brain and plasma concentrations following p.o. and i.v. dosing.

The following protocol was followed:

Male CD-1 mice were dosed with the compounds of Examples A-2 and A-3, either by i.v. administration (2 mg/kg) or by p.o. administration (10 mg/kg). Eight samples were taken for analysis in the i.v. leg at 2, 10, 30 min, 1, 2, 4, 8, and 24 (for i.v) and 9 samples in the p.o. leg at 15, 30 min, 1, 2, 4, 8, 24, 48 and 72 hrs.

The compounds of Examples A-2 and A-3 were both formulated in 10% DMSO/90% hydroxypropyl-beta-cyclodextrin (20% w/v in water) for i.v. and p.o. dosing. N =3 mice per time point.

Post dosing, terminal blood samples were taken from individual animals and delivered into labelled polypropylene tubes containing anticoagulant (EDTA). The samples were held on wet ice for a maximum of 30 min while sampling of all the animals in the cohort was completed. The blood samples were centrifuged for plasma (4° C., 21100 g for 5 min) and the resulting plasma transferred into corresponding labelled tubes. Terminal brains from each PO dosed animal were excised, rinsed with saline and placed into pre-weighed labelled polypropylene tubes and the samples re-weighed prior to storage.

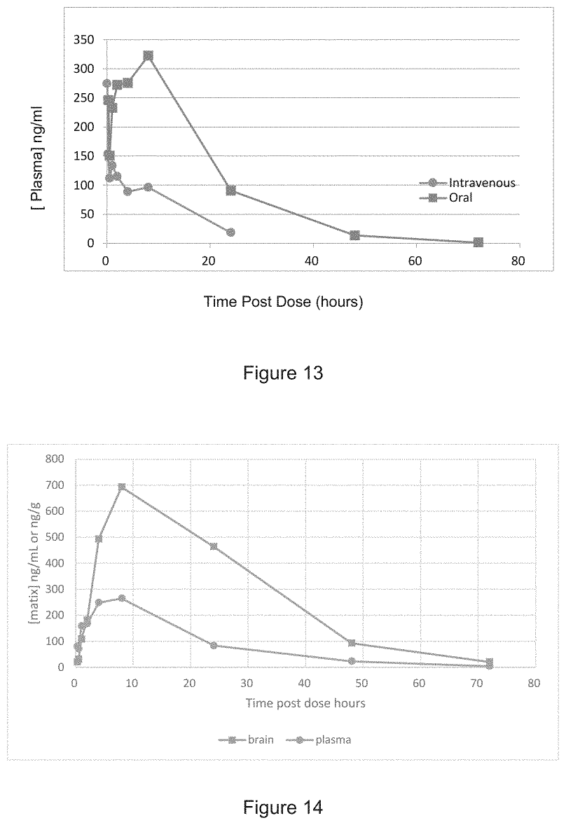

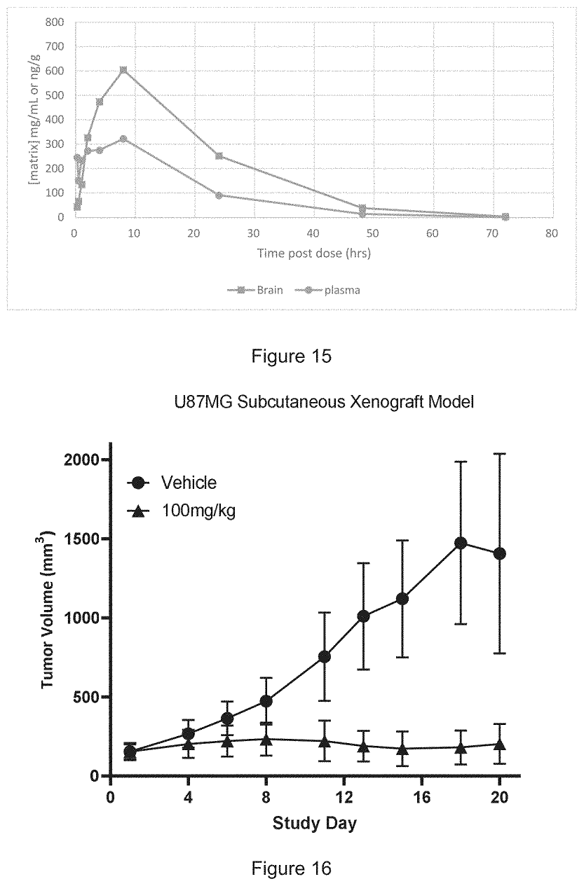

Quantitative bioanalysis was carried out using liquid chromatography—mass spectroscopy was performed. The results are shown in Tables 14, 15 and 16 below and in FIGS. 12 to 15.

Oral Bioavailability

TABLE 14
2 mq/Kq IV

| | | Atropisomer | Atropisomer |
|---|---|---|---|
| Parameter | Units | A-2 | A-3 |
| T ½ | Hr | 9.0 | 8.4 |
| CI | mL/min/kg | 20.5 | 16.8 |
| Cmax | ng/mL | 278 | 274 |
| AUCinf | ng · hr/mL | 1624 | 1987 |

TABLE 15
10 mq/Kq PO

| | | Atropisomer | Atropisomer |
|---|---|---|---|
| Parameter | Units | A-2 | A-3 |
| T ½ | Hr | 10.7 | 8.3 |
| Cmax | ng/mL | 265 | 322 |
| AUCinf | ng · hr/mL | 6131 | 6925 |
| F | % | 76 | 70 |

The results demonstrate that Atropisomers A-2 and A-3 are highly absorbed following oral dosing in mice.

Brain Exposure

TABLE 16

| | | Atropisomer | Atropisomer |
|---|---|---|---|
| Parameter | Units | A-2 | A-3 |
| T ½ | Hr | 12.1 | 8.2 |
| Tmax | Hr | 8.0 | 8.0 |
| Cmax | ng/mL | 693 | 604 |
| AUClast plasma | ng · hr/mL | 6131 | 6925 |
| AUClast brain | ng · hr/mL | 20528 | 14103 |

The results of the brain exposure studies presented in Table 16 demonstrate that atropisomers A-2 and A-3 both have high brain exposure. In the case of atropisomer A-2, the results demonstrate that atropisomer A-2 has high brain exposure with an AUC B:P ratio of 3.3 following oral dosing in mice.

H. In Vivo Efficacy

Atropisomer A-2 shows efficacy in glioblastoma mouse models when tumours are implanted subcutaneously and orthotopically, as indicated by the studies described below.

(i) In Vivo Anti-Cancer Activity in U87MG Subcutaneous Xenograft Model

Male athymic nude mice bearing U87MG tumours were given an oral dose of 100 mg/kg of atropisomer A-2 on days 1, 4 and 7 and the tumour volumes were measured over 20 days. Tumour volumes in a control group of tumour-bearing mice, who had received vehicle only at the same time points were also measured. The treated group showed significantly decreased tumour volume compared to control (3.85% T/C at day 13), as shown in FIG. 16.

(ii) In Vivo Anti-Cancer Activity in U87-Luc Orthotopic Xenograft Model

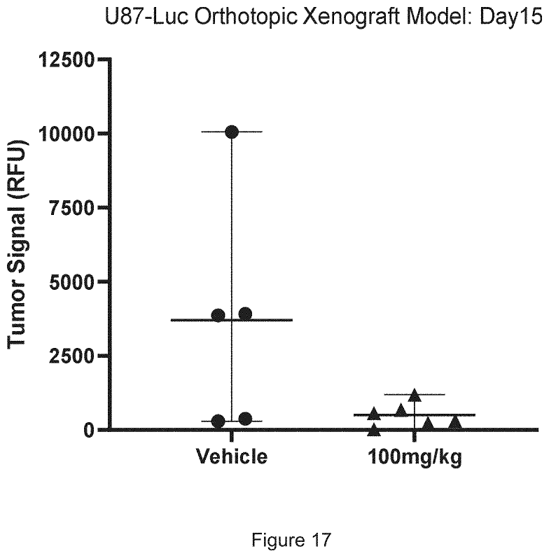

U87-Luc cells were intracerebrally implanted into the brains of male athymic nude mice and tumour growth was monitored by bioluminescent signal. In the treatment group animals were given an oral dose of 100 mg/kg of atropisomer A-2 on days 1, 4, 7, 10 and 13. The control group animals were given vehicle only. The results, shown in FIG. 17, demonstrate a decrease in tumour signal for the treated verses the control group on Day 15.

(iii) In Vivo Anti-Cancer Activity in Mice Bearing HCT116 Tumours

Atropisomer A-2 has shown efficacy in a KRAS mutated colorectal cancer model, as described below.

Male athymic nude mice bearing HCT116 xenograft tumours were give an oral dose of 100 mg/kg atropisomer A-2 on days 1, 8 and 15 and the tumour volumes were measured over 3 weeks. Tumour volumes in a control group of tumour-bearing mice, who had received vehicle only at the same time points were also measured.

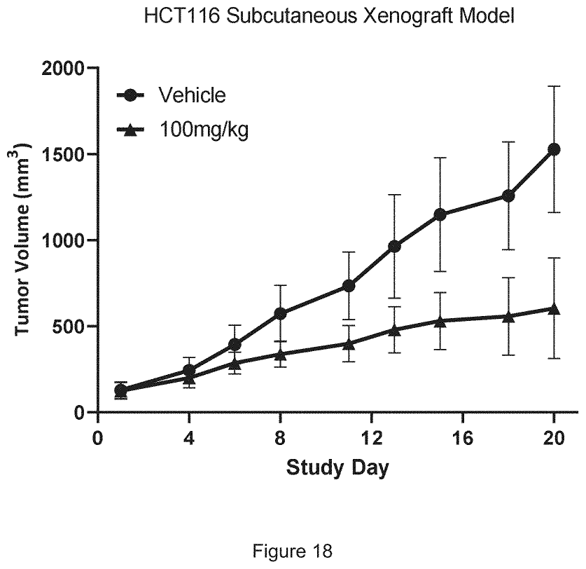

The results, shown in FIG. 18, demonstrate a pronounced effect on tumour growth at day 20 (TGI 60%).

Pharmaceutical Formulations

(i) Tablet Formulation

A tablet composition containing a composition of matter or an atropisomer of the invention is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a composition of matter or an atropisomer of the invention with 100 mg lactose and filling the resulting mixture into standard opaque hard gelatin capsules.

(iii) Injectable Formulation I

A parenteral composition for administration by injection can be prepared by dissolving a composition of matter or an atropisomer of the invention (e.g. in a salt form) in water containing 10% propylene glycol to give a concentration of active compound of 1.5% by weight. The solution is then sterilised by filtration, filled into an ampoule and sealed.

(iv) Injectable Formulation II

A parenteral composition for injection is prepared by dissolving in water a composition of matter or an atropisomer of the invention (e.g. in salt form) (2 mg/ml) and mannitol (50 mg/ml), sterile filtering the solution and filling into sealable 1 ml vials or ampoules.

(v) Injectable formulation III

A formulation for i.v. delivery by injection or infusion can be prepared by dissolving a composition of matter or an atropisomer of the invention (e.g. in a salt form) in water at 20 mg/ml. The vial is then sealed and sterilised by autoclaving.

(vi) Injectable formulation IV

A formulation for i.v. delivery by injection or infusion can be prepared by dissolving a composition of matter or an atropisomer of the invention (e.g. in a salt form) in water containing a buffer (e.g. 0.2 M acetate pH 4.6) at 20 mg/ml. The vial is then sealed and sterilised by autoclaving.

(vii) Subcutaneous Injection Formulation

A composition for sub-cutaneous administration is prepared by mixing a composition of matter or an atropisomer of the invention with pharmaceutical grade corn oil to give a concentration of 5 mg/ml. The composition is sterilised and filled into a suitable container.

(viii) Lyophilised formulation

Aliquots of formulated a composition of matter or atropisomer of the invention are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A composition of matter which:
(i) consists of at least 90% by weight of an atropisomer (2A) and 0-10% by weight of an atropisomer of formula (2B); or
(ii) consists of at least 90% by weight of an atropisomer (2B) and 0-10% by weight of an atropisomer of formula (2A);
wherein the atropisomer of formula (2A) and the atropisomer of formula (2B) are represented by: 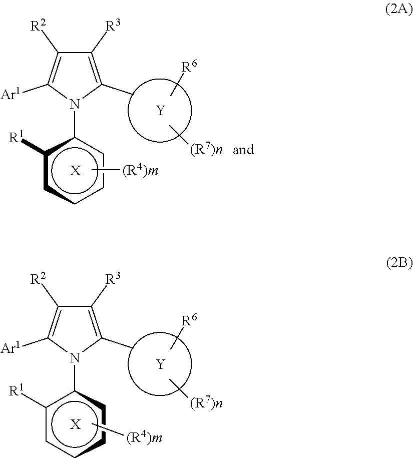
or are pharmaceutically acceptable salts or tautomers thereof, wherein:
ring X is a benzene or pyridine ring;
ring Y is selected from a benzene ring, a pyridine ring and a thiophene ring;
R¹ is trifluoromethyl;
R² is hydrogen;
R³ is hydrogen;
m is 0 or 1;
n is 0, 1 or 2;
R⁴ is selected from:
fluorine;
chlorine;
bromine; and
a C₁₋₄ alkyl group where 0 or 1 of the carbons in the alkyl group are replaced with a heteroatom 0, the alkyl group being optionally substituted with one or more fluorine atoms;
Ar¹ is a monocyclic aromatic ring selected from benzene and pyridine; each monocyclic aromatic ring being unsubstituted or substituted with 1 or 2 substituents R⁵;
R⁵ when present is selected from bromine; fluorine; chlorine; and cyano;
R⁷ is independently selected from R⁴;
R⁶ is a group Q¹-R^{a}—R^{b};
Q¹ is absent or is selected from CH₂, CH(CHs), C(CH₃)₂, cyclopropane-1,1-diyl and cyclobutane-1,1-diyl;
R^{a} is absent or is selected from O; C(O); C(O)O; CONR^{c}; N(R^{c})CO; N(R^{c})CONR^{c}; NR^{c}; and SO₂;
R^{b} is selected from:
a C₁₋₄ non-aromatic hydrocarbon group where 0 or 1 but not all of the carbon atoms in the hydrocarbon group are replaced with a heteroatom selected from N and O, the C₁₋₄ non-aromatic hydrocarbon group being optionally substituted with one or more substituents selected from fluorine and a group Cyc¹; and
a group Cyc¹;
R^{c} is selected from hydrogen and a C₁₋₄ non-aromatic hydrocarbon group;
Cyc¹ is a non-aromatic 4-7 membered heterocyclic ring group containing a nitrogen ring member and optionally second heteroatom ring member selected from N and O; the non-aromatic 4-7 membered heterocyclic ring group being optionally substituted with one or more substituents selected from hydroxyl; amino; mono-C₁₋₄ alkylamino; di-C₁₋₄ alkylamino; and a C₁₋₄ saturated hydrocarbon group where 0 or 1 but not all of the carbons in the hydrocarbon group are replaced with a heteroatom selected from N and O.

2. A composition of matter according to claim 1 wherein m is 0.

3. A composition of matter according to claim 1 wherein Ar¹ is a benzene ring optionally substituted with one or more substituent R⁵.

4. A composition of matter according to claim 3 wherein the benzene ring Ar¹ is unsubstituted or is substituted with 1 substituent R⁵.

5. A composition of matter according to claim 1 wherein R⁵, when present, is selected from fluorine, chlorine and cyano.

6. A composition of matter according to claim 1 wherein the ring Y is a benzene ring or a pyridine ring.

7. A composition of matter according to claim 1 wherein R⁶ is a group Q¹-R^{a}—R^{b}; and Q¹ is absent or is selected from CH₂, CH(CH₃), C(CH₃)₂, cyclopropane-1,1-diyl and cyclobutane-1,1-diyl.

8. A composition of matter according to claim 1 wherein R^{a} is CONR^{c}.

9. A composition of matter according to claim 1 wherein R^{b} is selected from:
a C₁₋₈ non-aromatic hydrocarbon group wherein 1 of the carbon atoms in the hydrocarbon group is replaced with a nitrogen heteroatom.

10. A composition of matter according to claim 1 wherein R^{c}, when present is hydrogen.

11. A composition of matter according to claim 1 wherein R⁶ is selected from the groups in the table below:

| | |
|---|---|
| 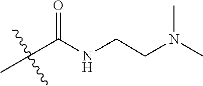 [SMILES: *C(=O)NCCN(C)C] | A |
| 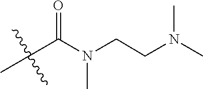 [SMILES: *C(=O)N(C)CCN(C)C] | B |
| 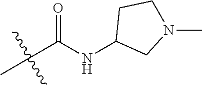 [SMILES: *C(=O)NC1CCN(C)C1] | C |
| 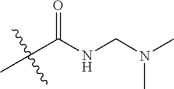 [SMILES: *C(=O)NCN(C)C] | D |
| 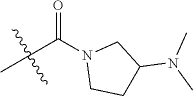 [SMILES: *C(=O)N1CCC(N(C)C)C1] | E |
| 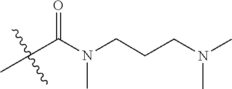 [SMILES: *C(=O)N(C)CCCN(C)C] | F |
| 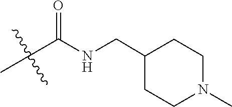 [SMILES: *C(=O)NCC1CCN(C)CC1] | G |
| 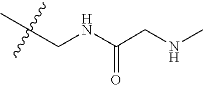 [SMILES: *CNC(=O)CNC] | H |
| 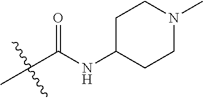 [SMILES: *C(=O)NC1CCN(C)CC1] | I |
| 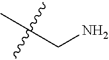 [SMILES: *CN] | J |
| 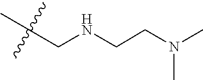 [SMILES: *CNCCN(C)C] | K |
| 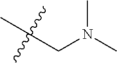 [SMILES: *CN(C)C] | L |
| 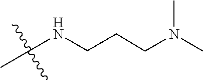 [SMILES: *NCCCN(C)C] | M |
| 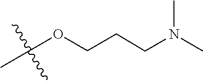 [SMILES: *OCCCN(C)C] | N |
| 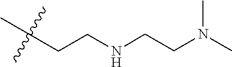 [SMILES: *CCNCCN(C)C] | O |
| 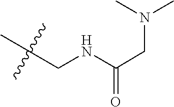 [SMILES: *CNC(=O)CN(C)C] | P |
| 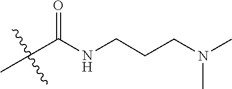 [SMILES: *C(=O)NCCCN(C)C] | Q |
| 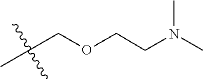 [SMILES: *COCCN(C)C] | R |
| 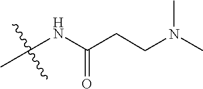 [SMILES: *NC(=O)CCN(C)C] | S |
| 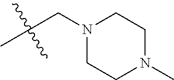 [SMILES: *CN1CCN(C)CC1] | U |
| 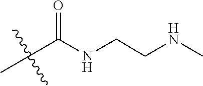 [SMILES: *C(=O)NCCNC] | X |
| 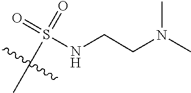 [SMILES: *S(=O)(=O)NCCN(C)C] | Y |
| 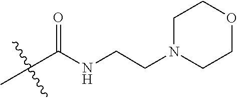 [SMILES: *C(=O)NCCN1CCOCC1] | Z |
| 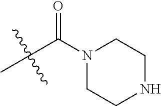 [SMILES: *C(=O)N1CCNCC1] | AA |
| 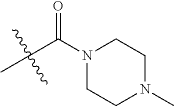 [SMILES: *C(=O)N1CCN(C)CC1] | AB |
| 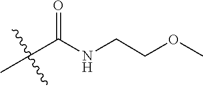 [SMILES: *C(=O)NCCOC] | AC |
| 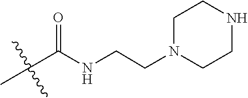 [SMILES: *C(=O)NCCN1CCNCC1] | AD |
| 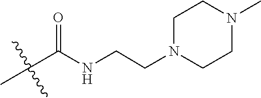 [SMILES: *C(=O)NCCN1CCN(C)CC1] | AE |
| 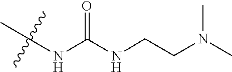 [SMILES: *NC(=O)NCCN(C)C] | AF |
| 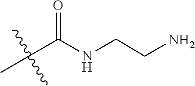 [SMILES: *C(=O)NCCN] | AG |
| 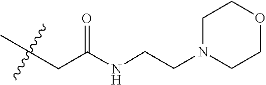 [SMILES: *CC(=O)NCCN1CCOCC1] | AI |
| 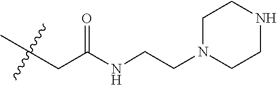 [SMILES: *CC(=O)NCCN1CCNCC1] | AJ |
| 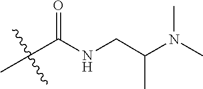 [SMILES: *C(=O)NCC(C)N(C)C] | AL |

12. A single atropisomer having a chemical structure as defined in claim 1, said single atropisomer being unaccompanied by any other atropisomer, or being accompanied by no more than 0.5% by weight relative to the single atropisomer of any other atropisomer.

13. A single atropisomer according to claim 12 which has an R-configuration about the bond linking ring X to the pyrrole nitrogen atom.

14. A single atropisomer according to claim 13, which has the R configuration represented by formula (1), or is a salt thereof:

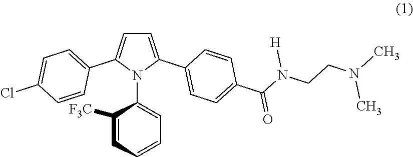

15. A (+)-L-tartaric acid salt of 2,4-[5-(4-chlorophenyl)-1-[2-(trifluoromethyl)-phenyl]pyrrol-2-yl]-N-[2 (dimethylamino)ethyl]benzamide having the formula (2):

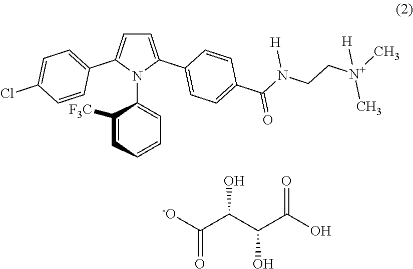

16. A pharmaceutical composition comprising a composition of matter according to claim 1 and a pharmaceutically acceptable excipient.

17. A method of treating a subject suffering from cancer, which method comprises administering to the subject a therapeutically effective amount of a composition of matter according to claim 1.

18. An invention as defined in any one of Embodiments 1.1 to 1.211, 2.1 to 2.15, 3.1 to 3.38, 4.1 to 4.12 and 5.1 to 5.9 herein.

19. A method of inhibiting PLK1 kinase or PLK4 kinase, which method comprises contacting the PLK1 kinase or the PLK4 kinase with a kinase inhibiting amount of a composition of matter according to claim 1.
